(19) United States
(12) Patent Application Publication
(10) Pub. No.: US 2026/0355253 A1
(43) Pub. Date: Nov. 12, 2026

(54) THERAPEUTICALLY ACTIVE CELLS AND EXOSOMES

(71) Applicants: Cedars-Sinai Medical Center, Los Angeles, CA (US); Capricor, Inc., Beverly Hills, CA (US)

(72) Inventors: Eduardo Marbán, Santa Monica, CA (US); Ahmed Ibrahim, Los Angeles, CA (US); Luis Rodriguez-Borlado, Manhattan Beach, CA (US); Jennifer J. Moseley, Northborough, MA (US); Chang Li, Harbor City, CA (US)

(21) Appl. No.: 19/740,962

(22) Filed: Jul. 14, 2026

Related U.S. Application Data
(62) Division of application No. 17/608,636, filed on May 7, 2020.
(60) Provisional application No. 62/845,228, filed on May 8, 2019.

(51) Int. Cl. A61K 35/34; A61K 35/33; A61P 9/00; C12N 5/077; G01N 33/50

(52) U.S. Cl. CPC A61K 35/34; A61K 35/33; A61P 9/00; C12N 5/0656; C12N 5/0657; G01N 33/5005; C12N 2501/415; C12N 2510/00; C12N 2510/04; G01N 2333/7055; G01N 2333/70596

(57) ABSTRACT

Several embodiments relate to methods of generating cells with therapeutic potency. Several embodiments relate to generating cells as a source of exosomes with therapeutic potency. The cells and exosomes with therapeutic potency are useful for repairing and/or regenerating damaged or diseased tissue, for example.

### CROSS-REFERENCE TO RELATED APPLICATION

This application is a divisional application of U.S. application Ser. No. 17/608,636, filed Nov. 3, 2021, which is the U.S. national phase application under 35 U.S.C. § 371 of International Application No. PCT/US2020/031808, filed May 7, 2020, which claims priority to U.S. Provisional Application No. 62/845,228, filed May 8, 2019. The content of each of these Related Applications in its entirety is incorporated herein by reference.

### STATEMENT REGARDING FEDERALLY SPONSORED R&D

This invention was made in part with government support under U.S. National Institutes of Health Grant No. R01HL124074 to Dr. Eduardo Marbán. The U.S. government may have certain rights in this invention.

### BACKGROUND

The present application relates generally to methods and compositions for the repair or regeneration of damaged or diseased cells or tissue. Several embodiments relate to administration of exosomes, such as exosomes engineered for high potency (or protein and/or nucleic acids from the exosomes) isolated from cells or synthetic surrogates in order to repair and/or regenerate damage or diseased tissues. In particular, several embodiments, relate to exosomes derived from certain cell types, such as for example cardiac stem cells and cells engineered for high therapeutic potency, such as fibroblast cells. Several embodiments relate to use of the exosomes in the repair and/or regeneration of cardiac tissue, for wound healing, and bone growth, for example.

Cardiosphere-derived cells (CDCs) trigger repair and functional improvement after injury to heart and skeletal muscle. Several early-stage clinical trials of CDCs have shown benefits on surrogate markers of disease progression in acquired or congenital forms of heart failure. Mechanistic preclinical studies reveal that CDCs exert their benefits indirectly, by secreting exosomes and other extracellular vesicles (EVs) that stimulate anti-inflammatory, antifibrotic, angiogenic, and cardiomyogenic pathways. Nevertheless, therapeutic potency remains inconsistent: CDCs and other primary cell types exhibit variable potency across donors, and process improvement efforts can also inadvertently undermine potency. Mechanistically-based strategies to increase potency are lacking, but highly desirable.

For cardiac applications of cell therapy, the gold standard potency assay measures functional and/or structural recovery in vivo after myocardial infarction (MI) in rodents. The continuing reliance on this costly, low-throughput model reflects a poor mechanistic understanding of the molecular determinants of potency. Here, high- and low-potency human CDCs were systematically compared at transcriptomic, translational, and functional levels. The insights not only include previously-unrecognized markers of CDC potency, but also strategies to enhance the therapeutic efficacy of CDCs, of other cell types, and of secreted exosomes.

### FIELD

Some embodiments relate to methods of generating high potency therapeutic cells or exosomes and the use of such high potency cells or exosomes for tissue repair and/or regeneration.

### DESCRIPTION OF RELATED ART

Many diseases, injuries and maladies involve loss of or damage to cells and tissues. Examples include, but are not limited to neurodegenerative disease, endocrine diseases, cancers, and cardiovascular disease. Just these non-limiting examples are the source of substantial medical costs, reduced quality of life, loss of productivity in workplaces, workers compensation costs, and of course, loss of life. For example, coronary heart disease is one of the leading causes of death in the United States, taking more than 650,000 lives annually. Approximately 1.3 million people suffer from a heart attack (or myocardial infarction, MI) every year in the United States (roughly 800,000 first heart attacks and roughly 500,000 subsequent heart attacks). Even among those who survive the MI, many will still die within one year, often due to reduced cardiac function, associated side effects, or progressive cardiac disease. Heart disease is the leading cause of death for both men and women, and coronary heart disease, the most common type of heart disease, led to approximately 400,000 deaths in 2008 in the US. Regardless of the etiology, most of those afflicted with coronary heart disease or heart failure have suffered permanent heart tissue damage, which often leads to a reduced quality of life.

Wound healing is a process in which skin and tissues underneath the skin repair themselves after injury. The stages of wound healing include hemostasis (blood clotting), inflammation, proliferation or growth of new tissue, and maturation or remodeling. The wound healing process is fragile and subject to interruption or failure, leading to chronic or non-healing wounds. As another example, bone formation, also known as ossification or osteogenesis, and bone growth occur during development, for example. Bone healing after fractures or strain, for example, requires repair, bone formation or ossification, and remodeling. Healing time may be delayed depending on injury or fracture location and patient age, for example.

### SUMMARY

There exists a need for methods and compositions to repair and/or regenerate tissue that has been damaged (or is continuing to undergo damage) due to injury, disease, or combinations thereof. While classical therapies such as pharmacological intervention or device-based intervention or surgery provide positive effects, there are provided herein methods and compositions that yield unexpectedly beneficial effects in the repair or regeneration of damaged or diseased tissues (though in some embodiments, these methods and compositions are used to complement classical therapies).

Provided herein is a method of preparing high potency therapeutic cells for treating conditions requiring tissue repair, tissue regeneration, or tissue growth, the method comprising activating Wnt/β-catenin signaling in low therapeutic potency cells by one or more of: overexpressing β-catenin in the low therapeutic potency cells, downregulating expression of one or more of mest, miR-335, EXTL1, CD90, and CD105 in the low therapeutic potency cells, upregulating expression of LRP5/6 in the low therapeutic potency cells, treating the low therapeutic potency cells with a modulator of β-catenin expression, and blocking GSK3β in the low therapeutic potency cells, to thereby generate high potency therapeutic cells having an increased therapeutic potency relative to the low therapeutic potency cells without activation of Wnt/β-catenin signaling, wherein the high potency therapeutic cells are effective for facilitating tissue repair, tissue regeneration, or tissue growth.

In some embodiments, the modulator of β-catenin expression is tideglusib or 6-bromoindirubin-3′-oxime (BIO). In some embodiments, activating Wnt/β-catenin signaling comprises increasing β-catenin expression in the low therapeutic potency cells by about 50% to about 300% relative to the low therapeutic potency cells without activation of Wnt/β-catenin signaling.

In some embodiments, the low therapeutic potency cells are fibroblast cells. Optionally, the fibroblast cells are genetically modified fibroblasts cells that overexpress gata4. Optionally, the genetically modified fibroblast cells have higher mRNA expression of gata4 relative to fibroblast cells that do not overexpress gata4 by a log 2 fold of about 0.2 to about 4. Optionally, the method further comprises genetically modifying fibroblast cells to overexpress gata4.

In some embodiments, the low therapeutic potency cells are low therapeutic potency cardiosphere-derived cells (CDCs). Optionally, the low therapeutic potency cells are immortalized CDCs. Optionally, the method further comprising immortalizing CDCs to generate the immortalized CDCs. Optionally, the CDCs have a high therapeutic potency prior to being immortalized.

In some embodiments, the method further comprises determining a population of cells as having low therapeutic potency. Optionally, determining comprises measuring an expression level of one or more Wnt/β-catenin signaling mediators and regulators in the population of cells. In some embodiments, the one or more Wnt/β-catenin signaling mediators and regulators are specific to canonical Wnt/β-catenin signaling. In some embodiments, the one or more Wnt/β-catenin signaling mediators and regulators is selected from: β-catenin, LRP5/6, mest, and EXTL1. In some embodiments, determining comprises measuring an mRNA level of one or more non-canonical Wnt signaling mediators. In some embodiments, the one or more non-canonical Wnt signaling mediators is selected from: ror2, nfatc2, axin2, rac2, and apcdd1.

In some embodiments, the low therapeutic potency cells are allogeneic to a subject in need of treating a condition requiring the tissue repair, tissue regeneration, or tissue growth. In some embodiments, the low therapeutic potency cells are autologous to a subject in need of treating a condition requiring the tissue repair, tissue regeneration, or tissue growth.

In some embodiments, the method further comprises isolating exosomes from the high potency therapeutic cells, wherein the exosomes are effective for facilitating tissue repair, tissue regeneration, or tissue growth.

In some embodiments, the high potency therapeutic cells are effective for one or more of reducing cardiac scar size, increasing myocardial infarct wall thickness, increasing ejection fraction, reducing mortality from myocardial infarction, increasing exercise capacity, reducing skeletal muscle fibrosis, and increasing myofiber size, when administered to a subject in need of treating a condition requiring tissue repair, tissue regeneration, or tissue growth. In some embodiments, the increased therapeutic potency comprises a difference in a percentage therapeutic effect between the high potency therapeutic cells and the low therapeutic potency cells of about 5% to about 40%.

Also provided herein is a method of preparing high therapeutic potency exosomes for treating conditions requiring tissue repair, tissue regeneration, or tissue growth, the method comprising: providing a population of engineered high potency therapeutic cells having activated Wnt/β-catenin signaling, wherein the high potency therapeutic cells exhibit one or more of: upregulated β-catenin expression; downregulated levels of mest expression; upregulated levels of LRP5/6 expression; and downregulated levels of extl1 expression, relative to a population of low therapeutic potency cells; and isolating exosomes from the population, to thereby generate high therapeutic potency exosomes having an increased therapeutic potency relative to low therapeutic potency exosomes isolated from the low therapeutic potency cells without the activated Wnt/β-catenin signaling, wherein the high therapeutic potency exosomes are effective for facilitating tissue repair, tissue regeneration, or tissue growth. Optionally, the engineered high potency therapeutic cells comprise β-catenin expression that is higher by about 50% to about 300% relative to the low therapeutic potency cells.

In some embodiments, the engineered high potency therapeutic cells are engineered fibroblast cells. Optionally, the engineered fibroblast cells are genetically modified fibroblast cells that overexpress gata4. In some embodiments, the genetically modified fibroblast cells have higher expression of gata4 relative to fibroblast cells that do not overexpress gata4 by a log 2 fold of about 0.2 to about 4.

In some embodiments, the engineered high potency therapeutic cells are high therapeutic potency cardiosphere-derived cells (CDCs). Optionally, the engineered high potency therapeutic cells are high therapeutic potency immortalized CDCs.

In some embodiments, providing the population comprises: identifying low therapeutic potency cells; and activating Wnt/β-catenin signaling in the low therapeutic potency cells by one or more of: overexpressing β-catenin in the low therapeutic potency cells, downregulating expression of one or more of mest, miR-335, EXTL1, CD90, and CD105 in the low therapeutic potency cells, upregulating expression of LRP5/6 in the low therapeutic potency cells, treating the low therapeutic potency cells with a modulator of β-catenin expression, and blocking GSK3β in the low therapeutic potency cells, to thereby generate a population of cells enriched in the engineered high potency therapeutic cells. Optionally, the modulator of β-catenin expression is tideglusib or 6-bromoindirubin-3′-oxime (BIO).

In some embodiments, the low therapeutic potency cells are fibroblast cells. In some embodiments, the fibroblast cells overexpress gata4. In some embodiments, the method further comprises genetically modifying fibroblast cells to overexpress gata4.

In some embodiments, the low therapeutic potency cells are immortalized CDCs. Optionally, the method further comprises immortalizing CDCs to generate the immortalized CDCs. Optionally, the CDCs have a high therapeutic potency prior to being immortalized.

In some embodiments, the population of cells are allogeneic to a subject in need of treating a condition requiring the tissue repair, tissue regeneration, or tissue growth. In some embodiments, the population of cells are heterologous to a subject in need of treating a condition requiring the tissue repair, tissue regeneration, or tissue growth.

In some embodiments, the high therapeutic potency exosomes are effective for one or more of reducing cardiac scar size, increasing myocardial infarct wall thickness, increasing ejection fraction, reducing mortality from myocardial infarction, increasing exercise capacity, reducing skeletal muscle fibrosis, and increasing myofiber size, when administered to a subject in need of treating a condition requiring tissue repair, tissue regeneration, or tissue growth. In some embodiments, the increased therapeutic potency comprises a difference in therapeutic effect measured in percentage between the high potency therapeutic exosomes and exosomes isolated from low therapeutic potency cells of about 5% to about 40%.

Described herein, in some embodiments, are methods of preparing high potency therapeutic cells for treating conditions requiring tissue regeneration, tissue repair, or tissue growth, the method comprising activating Wnt/β-catenin signaling in low therapeutic potency cells, wherein the therapeutic potency of the low therapeutic potency cells is increased following activation of Wnt/β-catenin signaling relative to therapeutic potency before activation of Wnt/β-catenin signaling, wherein the high potency therapeutic cells are effective for facilitating tissue regeneration, tissue repair, or tissue growth. In some embodiments, activation of Wnt/β-catenin comprises overexpressing β-catenin in the low therapeutic potency cells, treating the low therapeutic potency cells with a modulator of β-catenin expression, blocking GSK3β, genetic ablation of GSK3β, or knockdown of GSK3β. In some embodiments, the methods described herein further comprise overexpressing gata4. In some embodiments, treatment of low therapeutic potency cells with a modulator of β-catenin expression comprises upregulation of β-catenin expression. In some embodiments, the modulator of β-catenin expression is 6-bromoindirubin-3′-oxime (BIO) or tideglusib. In some embodiments, activation of Wnt/β-catenin signaling comprises alterations of nucleic acid and/or protein expression. In some embodiments, alterations of nucleic acid and/or protein expression activation comprise downregulation of mest, downregulation of miR335, downregulation of EXTL1, downregulation of CD90, downregulation of CD105, upregulation of LRP5/6, upregulation of miR-92a, or combinations thereof. In some embodiments, the low therapeutic potency cells are cardiosphere-derived cells or fibroblast cells. In some embodiments, the conditions comprise muscular disorders, myocardial infarction, cardiac disorders, myocardial alterations, muscular dystrophy, fibrotic disease, inflammatory disease, or wound healing. In some embodiments, the tissue growth comprises bone growth.

Described herein, in some embodiments, are methods of preparing high therapeutic potency exosomes for treating conditions requiring tissue regeneration, tissue repair, or tissue growth, the methods comprising: (a) preparing high potency therapeutic cells by any of the methods disclosed herein; (b) collecting exosomes from the high potency therapeutic cells, wherein the high potency therapeutic cells are effective for facilitating tissue regeneration, tissue repair, or tissue growth. In some embodiments, the high therapeutic potency exosomes comprise increased levels of miR-92a, increased levels miR-146a, decreased levels of miR-199b, or combinations thereof. In some embodiments, the conditions comprise muscular disorders, myocardial infarction, cardiac disorders, myocardial alterations, muscular dystrophy, fibrotic disease, inflammatory disease, or wound healing. In some embodiments, the tissue growth comprises bone growth.

Described herein, in some embodiments, are methods of treating conditions requiring tissue regeneration, tissue repair, or tissue growth, comprising administering to a subject in need thereof high potency cells prepared by any of the methods disclosed herein. In some embodiments, administration of high potency cells alters gene expression and/or protein expression. In some embodiments, alteration of gene expression and/or protein expression comprises downregulation of bmp-3, downregulation of bmp-4, downregulation of GDF6, downregulation of GDF10, upregulation of bmp-2, upregulation of bmp-2r, upregulation of bmp-6, upregulation of bmp-8a, or combinations thereof.

Described herein, in some embodiments, are methods of treating conditions requiring tissue regeneration, tissue repair, or tissue growth, comprising administering to a subject in need thereof high potency exosomes prepared by any of the methods disclosed herein. In some embodiments, administration of high therapeutic potency exosomes alters gene expression. In some embodiments, alteration of gene expression comprises downregulation of bmp-3, downregulation of bmp-4, downregulation of GDF6, downregulation of GDF10, upregulation of bmp-2, upregulation of bmp-2r, upregulation of bmp-6, upregulation of bmp-8a, or combinations thereof.

Described herein, in some embodiments, are populations of enhanced potency exosomes, comprising: a plurality of exosomes for use in treating damaged or diseased tissue, wherein the exosomes are obtained from a population of source cells, wherein the source cells comprises CDCs or fibroblasts, wherein the source cells were exposed to a modulator of β-catenin expression that results in upregulation of β-catenin expression, and wherein the enhanced potency exosomes express miR-92a and/or miR-146a at greater levels as compared to exosomes obtained from source cells not exposed to the modulator of β-catenin expression.

Described herein, in some embodiments, are populations of cells engineered for enhanced therapeutic potency for use in treating damaged or diseased tissue, comprising: (a) upregulated β-catenin expression; (b) downregulated levels of mest expression; (c) upregulated levels of LRP5/6 expression; (d) downregulated levels of extl1 expression; (e) upregulated levels of miR-92a; or any combination thereof, relative to a population of low therapeutic potency source cells. In some embodiments, the population of low therapeutic potency source cells comprises CDCs or fibroblasts.

Described herein, in some embodiments, are populations of enhanced potency exosomes, comprising: a plurality of exosomes for use in treating damaged or diseased tissue, wherein the plurality of exosomes is obtained from a population of cells engineered for enhanced therapeutic potency as disclosed herein. In some embodiments, the plurality of exosomes comprises upregulated miR-92a and/or upregulated miR-146a relative to low therapeutic potency exosomes. In some embodiments, the enhanced potency exosomes are enriched for expression of one or more of ITGB1, CD9, and CD63, and are depleted for expression of HSC70 and/or GAPDH. In some embodiments, the enhanced potency exosomes are enriched for expression of one or more of ITGB1, HSC70, and GAPDH, and are depleted for CD9 expression.

Also provided herein is a use of a population of cells engineered for enhanced therapeutic potency, as disclosed herein, or a population of enhanced potency exosomes, as disclosed herein, to treat damaged or diseased tissue. Also provided is a use of a population of cells engineered for enhanced therapeutic potency, as disclosed herein, or a population of enhanced potency exosomes, as disclosed herein, in the preparation of a medicament for treatment of damaged or diseased tissue. In some embodiments, the damaged or diseased tissue comprises muscle tissue. In some embodiments, the muscle tissue comprises cardiac or skeletal muscle.

Also provided herein is a method of determining a therapeutic potency of a population of cells, comprising: measuring an expression level of one or more Wnt/β-catenin signaling mediators and regulators in a population of cells; and determining the population of cells has high or low therapeutic potency based on the measured level of the one or more Wnt/β-catenin signaling mediators and regulators. In some embodiments, the determining comprises comparing the measured level of the one or more Wnt/β-catenin signaling mediators and regulators to a reference level or reference range. In some embodiments, the reference range is a range of levels of the one or more Wnt/β-catenin signaling mediators and regulators in a population of cells having low or high therapeutic potency. In some embodiments, the one or more Wnt/β-catenin signaling mediators and regulators includes, without limitation, one or more of β-catenin, LRP5/6, mest, and EXTL1.

In some embodiments, the method further comprises measuring an mRNA level of one or more non-canonical Wnt signaling mediators. In some embodiments, the method comprises determining the population of cells has high or low therapeutic potency based on the measured level of the one or more Wnt/β-catenin signaling mediators and regulators, and the measured level of the one or more non-canonical Wnt signaling mediators.

In some embodiments, the population of cells is derived from a source of cells having variable therapeutic potency. In some embodiments, the population of cells comprises fibroblasts or CDCs.

### BRIEF DESCRIPTION OF THE DRAWINGS

FIGS. 1A-1F illustrate therapeutic efficacy of various human CDC cell lines. FIG. 1A shows changes in global heart function upon administering human CDC cell lines. FIG. 1B shows transcriptomic comparison of HP and LP CDC. FIG. 1C shows enrichment of non-canonical Wnt pathway members in LP CDCs. FIGS. 1D, 1E, and 1F show that little difference was evident in molecules shared by canonical and non-canonical Wnt signaling pathways (Frizzled receptors, Dishevelled) and Wnt ligands.

FIGS. 2A-2K illustrate that β-catenin enhances CDC potency. FIG. 2A show a correlation between total β-catenin levels in donor CDCs (n=13) and therapeutic performance (expressed as change in left ventricular ejection fraction) in vivo. FIG. 2B shows higher expression of the Wnt coreceptor LRP5/6 in high-potency CDCs (HP) compared with low-potency CDCs (LP; n=5 per group). FIG. 2C shows exposing LP CDCs to 5 μM BIO significantly increased β-catenin levels. FIGS. 2D, 2E, 2F, and 2G shows exposing LP CDCs to 5 μM BIO restored therapeutic efficacy (n=6 per group;). Percent scar was determined using image J quantification from Masson trichrome stained sections. These results were further confirmed in CDCs from a low potency lot from a sometimes-potent CDC source (LPL), as BIO exposure restored potency to levels similar to potent lots from the same donor (n=5 per group; FIGS. 2H and 2I). Restoration of β-catenin levels also rescued potency in CDCs that were immortalized (SV40-T+t) with diminished potency (imCDC) (n=7 per group; FIGS. 2J and 2K). Statistical analysis: *p<0.05, **p<0.01, ***p<0.001, 95% CI using Student's Independent t-test.

FIGS. 3A-3J illustrate mest regulation of β-catenin in CDCs. FIG. 3A shows the experimental schematic. RNA from three pairs of cells was sequenced: CDCs from a low-potency donor (LP), CDCs from a low-potency lot from an otherwise potent donor (LPL), and CDCs with diminished potency due to immortalization (imCDC). Differential expression analysis was made within each group (BIO exposed versus vehicle control) and results (expressed in fold change) were averaged among the three groups. FIG. 3B shows that sequencing identified the β-catenin regulator mesoderm specific transcript (mest) and its cognate micro RNA (miR-335) are downregulated. FIG. 3C shows qPCR validation of the changes in mest and miR-335. FIG. 3D shows fold change in gene expression of miR-335 in extracellular vesicles (EVs) isolated from LP, LPL, and imCDC exposed to BIO compared with their vehicle control counterparts. FIG. 3E showsEVs from highly potent CDC EVs decrease mest in fibroblasts. FIGS. 3F, 3G and 3H shows qPCR verification of the Wnt signaling co-receptor, LRP5/6, and a member of the exostosin family glucosyltransferases EXTL1 in BIO-exposed LP. LPL, and imCDCs. FIG. 3I shows verification of EXTL1 protein downregulation in LP cells following BIO exposed. FIG. 3J shows flow cytometry of BIO exposure to LP increased LRP5/6 level. Statistical analysis: *p<0.05, **p<0.01, ***p<0.001, 95% CI using Student's independent t-test.

FIGS. 4A-4D illustrate mest inhibition in immortalized CDCs. FIG. 4A shows lentiviral transduction of SV40 T+t transgene leads to immortalization but attenuation of β-catenin levels and therapeutic efficacy in vivo as β-catenin ELISA and change in left ventricular functional improvement (ΔEF) in a mouse MI model. FIG. 4B shows Western blot and pooled data of EXTL1 and mest protein levels in primary CDCs (pCDC) and modified immortalized CDCs (imCDCsh-mest). FIG. 4C shows increased lrp5/6 in imCDCsh-mest compared with pCDC by flow cytometry (n=two replicates per group). FIG. 4D shows successful maintenance of β-catenin protein levels over several passages after immortalization is coupled with a small hairpin-mediated knockdown of mest (n=three replicates per group). The dotted line at 40 ng/μl represents the mean β-catenin level among highly potent donors. FIG. 4E shows qPCR of miR146a and miR199b in EVs of pCDC and imCDCsh-mest. Performance of imCDCsh-mest and pCDC in mouse models of acute MI (n=7 per group), including structural improvement (FIGS. 4F, 4G, and 4H) and functional improvement (FIG. 4I). Statistical analysis: *p<0.05, **p<0.01, ***p<0.001, 95% CI using Student's Independent t-test.

FIGS. 5A-5I illustrate NHDF immortalization with β-catenin or β-catenin/gata4. FIG. 5A shows qPCR verification of β-catenin or β-catenin/gata4 in the transduced cells. FIG. 5B shows cell morphology changed after transduction. NHDFβcat and NHDFβcat/gata4 became more endothelial-like and epithelial-like, respectively. FIG. 5C shows flow cytometry of CD90, CD105, and lrp5/6 in NHDF, NHDFβcat and NHDFβcat/gata4 (n=3 replicates per group). FIG. 5D shows ELISA of β-catenin level after transduction (n=3 replicates per group). FIG. 5E shows qPCR of microRNA markers in the extracellular vesicles of transduced cells (n=3 replicates per group; only 1 of the 3 replicates in miR199b was able to detect CT value). FIGS. 5F, 5G, 5H, and 5I show mortality is enhanced in myocardial infarction mice injected with NHDFs. However, animals given NHDFs transduced with β-catenin or β-catenin and gata4 leads to improved mortality, functional improvement and attenuation of remodeling like those observed in CDCs and CDC EVs. Scale bar: 100 μm. Statistical analysis: *p<0.05, **p<0.01, ***p<0.001, 95% CI using Student's Independent t-test.

FIGS. 6A-6E illustrate bioactivity of ASTEX in an mdx mouse model of Duchenne muscular dystrophy. FIG. 6A shows a schematic of the experimental design. Mice underwent graded exercise testing, then were injected with ASTEX or vehicle control (IMDM) into the femoral vein. Exercise testing was repeated 3 weeks later. FIG. 6B shows maximal exercise capacity was significantly improved in ASTEX-injected mdx mice after 3 weeks (n=5-6 per group). FIG. 6C shows representative Masson's trichrome stained micrographs from vehicle and ASTEX-injected mdx TA muscles. Pooled data from c indicate less muscle fibrosis in mdx TA muscles three weeks after ASTEX injection (n=5 per group). Scale bars: 100 μm. FIG. 6D shows pooled data from 1,000 analyzed myofibers per muscle in FIG. 6E indicate ASTEX shifted the myofiber size distribution to larger diameters (n=5 per group). Statistical analysis: *p<0.05, **p<0.01, ***p<0.001, 95% CI using Student's Independent t-test.

FIGS. 7A-7H illustrate that β-catenin-activation leads to downstream activation of bmp2 in target cells via miR-92a. FIG. 7A shows a heat map of differentially expressed genes in neonatal rat ventricular myocytes exposed to HP EVs compared to control. FIG. 7B shows upregulation of anti-fibrotic and downregulation of pro-fibrotic members of the bmp family members in HP EV-exposed myocytes. FIG. 7C shows enrichment of miR-92a in HP-EVs compared to LP EVs (n=three donors EVs/group). FIG. 7D shows exposure of fibroblasts to EVs from HP cells leads to increased bmp2 expression (n=3 replicates per group). FIGS. 7E and 7F shows that consistent with potency, EVs isolated from imCDC^{shmest} and ASTEX are enriched in miR-92a compared to primary CDC EVs and fibroblast EVs respectively. FIG. 7G shows mest is the turning point between non-canonical Wnt and canonical Wnt signal pathway, which is a determinant for therapeutic cell potency. FIG. 7H shows a schematic of mechanism of action according to some embodiments. β-catenin activation in CDCs leads to enrichment of miR-92a in secreted EVs. Secreted EVs are taken up by target cells, activate bmp2 signaling leading to healing and repair. *p<0.05, **p<0.01, ***p<0.001, 95% CI using Student's Independent T-test.

FIGS. 8A-8E illustrate β-catenin levels in HP-CDCs and LP-CDCs cells. FIG. 8A shows the β-catenin profile of the cardiosphere process where CDCs are made from EDCs (n=3 replicates per group). FIG. 8B shows beta catenin ELISA of CDCs exposed to increasing concentrations of BIO. FIG. 8C shows flow cytometry of CD90, CD105 and DDR2 in BIO-exposed LP cells. FIGS. 8D and 8E show that BIO, a reversible inhibitor of GSK3β (and activator of β-catenin) showed a more rapid decay of effect than the irreversible inhibitor tideglusib (n=3 replicates per group). *p<0.05, **p<0.01, ***p<0.001, 95% CI using Student's Independent T-test.

FIG. 9A-9F illustrate the role of β-catenin in enhancing potency. FIG. 9A shows cell persistence of BIO-exposed LP CDCs compared to vehicle-exposed cells three weeks post-injection in infarcted mice (n=4-5 animals per group). Standard curve showing copy numbers of mage al (human-specific X-chromosome marker) in known numbers of CDCs (from the same LP donor used here) per 1 mg of cardiac tissue (left panel). CDCs treated with BIO were completely cleared from host tissue by three weeks post-injection (right panel). Differential expression of mRNA (FIGS. 9B and 9C) and micro RNAs (FIGS. 9D and 9E) in BIO-exposed CDCs compared to vehicle-exposed counterparts. Data represents average decreased (FIGS. 9B and 9D) and increased (FIGS. 9C and 9E) across all three BIO-exposed pairs. FIG. 9F shows activation of β-catenin in fibroblasts does not decrease mest contrary to β-catenin activation in CDCs (n=3 replicates per group). Scale bar: 100 μm. *p<0.05, **p<0.01, ***p<0.001, 95% CI using Student's Independent T-test.

FIGS. 10A and 10B illustrate exosome concentration and distribution from CDCs treated with BIO or vehicle control. FIG. 10A shows nanosight tracking analysis plots of extracellular vesicles (EVs) derived from LP, LPL, and imCDCs exposed to either vehicle control (DMSO) or 5 μM of BIO prior to serum-free conditioning. FIG. 10B shows expression of therapeutic miRs in the EVs of BIO-exposed LP CDCs compared to vehicle-exposed counterparts.

FIGS. 11A-11F illustrate traditionally immortalized CDCs. FIG. 11A shows morphology of CDCs after immortalization using simian virus 40 large and small T antigen knock-in (passage 7). FIG. 11B shows marker expression remains largely conserved with the exception of the negative potency marker CD90. FIG. 11C shows EV size distribution is conserved while EV output is increased post immortalization. FIG. 11D EV concentration is increased in immortalized CDCs compared to primary parent CDCs. FIG. 11E shows downregulation of therapeutically potent EV cargo including miR-146a and miR-210. FIG. 11F shows limitations in growth and viability of immortalized CDCs exposed to BIO compared with vehicle. Scale bar: 100 μm. *p<0.05, **p<0.01, ***p<0.001, 95% CI using Student's Independent T-test.

FIGS. 12A-12D illustrate attempts at engineering therapeutic potency. FIG. 12A shows gene expression of GSK3β and β-catenin of CDCs immortalized and coupled with GSK3β knockdown (^{imCDCsh-gsk3b}; n=3 replicates per group). FIG. 11B shows β-catenin ELISA comparison between pCDC and imCDC^{sh-gsk3b} (n=3 replicates per group). FIG. 11C shows phase contrast images of primary CDCs and CDCs immortalized with additional knockdown of mest (imCDC^{sh-mest}). ImCDCs exhibited increased projections and filopodia. FIG. 11D shows that pCDC and imCDC^{sh-mest} show significant differences in marker profile. FIG. 11E shows qPCR verification of mest, ext1, and extl1 in imCDC^{sh-mest} transduction (n=3 replicates per group). Scale bar: 100 μm. *p<0.05, **p<0.01, ***p<0.001, 95% CI using Student's Independent T-test.

FIGS. 13A and 13B illustrate production of EVs by imCDC^{sh-mest} FIG. 13A shows NanoSight tracking analysis size distribution of primary CDCs and imCDC^{sh-mest} FIG. 13B shows EV output from primary CDCs and imCDC^{sh-mest} Scale bar: 100 μm.

FIG. 14A illustrates qPCR comparison of telomerase expression in NHDF, NHDFβcat, and NHDFβcat/gata4 (n=3 replicates per group). FIG. 14B shows that cell morphology changed to smooth muscle cell-like after β-catenin-etv2 transduction in NHDF. FIG. 14C shows NanoSight tracking analysis plots of EVs derived from NHDF, NHDFβcat, and NHDFβcat/gata4. (n=3 replicates per group). Scale bar: 100 μm. *p<0.05, **p<0.01, ***p<0.001, 95% CI using Student's Independent T-test.

FIG. 15A illustrates the effect of canonical wnt signaling activation (BIO), inhibition (JW67), or control in a mouse model of acute myocardial infarction. Upregulation (BIO) or inhibition (JW67) of β-catenin have modest effects on functional improvement in the mouse MI model (n=6-8 animals per group). FIGS. 15B and 15C shows that CDC EVs trigger cardiomyocyte proliferation in vitro. *p<0.05, **p<0.01, ***p<0.001, 95% CI using Student's Independent T-test.

FIG. 16 shows a schematic diagram of a method of preparing high potency therapeutic cells for treating conditions requiring tissue repair, tissue regeneration, or tissue growth, according to embodiments of the present disclosure.

FIG. 17 shows a schematic diagram of a method of preparing high therapeutic potency exosomes for treating conditions requiring tissue repair, tissue regeneration, or tissue growth, according to embodiments of the present disclosure.

FIGS. 18A-18E illustrate the therapeutic potency of immortalized CDC (imCDC^{sh-mest})-derived exosomes in a model of Duchenne Muscular Dystrophy (DMD). FIG. 18A shows a study design of a mdx transgenic mouse study for therapeutic potency of immortalized CDC (imCDC^{sh-mest})-derived exosomes. FIGS. 18B, 18C, 18D and 18E illustrate muscle force measurement in mdx mice at the indicated number of weeks after intravenous injection of immortalized CDC (imCDC^{sh-mest})-derived exosomes or vehicle.

FIG. 19 shows surface marker characterization (for certain selected markers) of immortalized CDC (imCDC^{sh-mest})-derived exosomes (IMEX) and ASTEX.

### DETAILED DESCRIPTION

Methods of preparing high potency therapeutic cells and/or high therapeutic potency exosomes for treating conditions requiring tissue repair, tissue regeneration, or tissue growth are provided. In general terms, high potency therapeutic cells of the present disclosure exhibit patterns of gene and/or protein expression level consistent with a higher level of canonical Wnt signaling (e.g., Wnt/β-catenin signaling) compared to low potency therapeutic cells. In some embodiments, the high potency therapeutic cells exhibit patterns of gene and/or protein expression level consistent with a reduced level of non-canonical Wnt signaling compared to low potency therapeutic cells. In some embodiments, high potency therapeutic cells of the present disclosure exhibit patterns of gene and/or protein expression level consistent with preferential activation of canonical Wnt signaling over non-canonical Wnt signaling. The high potency therapeutic cells of the present disclosure can have an increased therapeutic potency relative to the low therapeutic potency cells. In some embodiments, the method includes isolating exosomes from the high potency therapeutic cells, to thereby generate high therapeutic potency exosomes. In some embodiments, high therapeutic potency exosomes isolated from the high potency therapeutic cells an increased therapeutic potency relative to low therapeutic potency exosomes isolated from the low therapeutic potency cells. The high potency therapeutic cells and/or high therapeutic potency exosomes can be effective for facilitating tissue repair, tissue regeneration, or tissue growth.

Several embodiments of the methods and compositions disclosed herein are useful for the treatment of tissues that are damaged or adversely affected by disease(s). The vast majority of diseases lead to at least some compromise (even if acute) in cellular or tissue function. Several embodiments of the methods and compositions disclosed herein allow for repair and/or regeneration of cells and/or tissues that have been damaged, limited in their functionality, or otherwise compromised as a result of a disease. In several embodiments, methods and compositions disclosed herein may also be used as adjunct therapies to ameliorate adverse side effects of a disease treatment that negatively impacts cells or tissues. As used herein, “treat” or “treatment” refer to curing, preventing occurrence of, ameliorating, preventing deterioration of, and/or slowing the progress of a condition or disease.

### Wnt Signaling Pathways

Wnt signaling pathways are a group of signal transduction pathways which begin with proteins that pass signals into a cell through cell surface receptors. Canonical and non-canonical Wnt signaling pathways are known. Both canonical and non-canonical Wnt signaling pathways are activated by the binding of a Wnt-protein ligand to a Frizzled family receptor, with biological signals passing to the Dishevelled protein inside the cell. The canonical Wnt pathway leads to regulation of gene transcription, while non-canonical pathways regulate the cytoskeleton and intracellular calcium, for example. Canonical Wnt signaling pathways involve β-catenin. By contrast, non-canonical Wnt signaling operates independent of β-catenin.

### Bone Morphogenetic Proteins (BMPs)

Bone morphogenetic proteins (BMPs) comprise a group of growth factors or cytokines that are members of the TGF-beta superfamily. BMPs play a role in various physiological processes, including the formation of bone and cartilage, orchestration of tissue architecture throughout the body, wound healing, and pathological conditions such as cancer, esophagitis, Barrett's esophagus, and adenocarcinoma of the gastrointestinal tract, for example. The BMP subfamily comprises at least 20 members, including bmp-1, bmp-2, bmp-3, bmp-4, bmp-5, bmp-6, bmp-7, bmp-8a, bmp-8b, bmp-10, and bmp-15. The BMP receptors (BMPRs) are transmembrane serine/threonine kinases that include type I receptors BMPR1A and BMPR1B and the type II receptor BMPR2. Signal transduction occurs through the formation of heteromeric complexes of type I receptors and type II receptors. BMP signaling can occur through NF-KB, p38, and JNK via TAK1 and TAB1/2, through SMAD proteins, and/or through PKA, for example.

### Methods

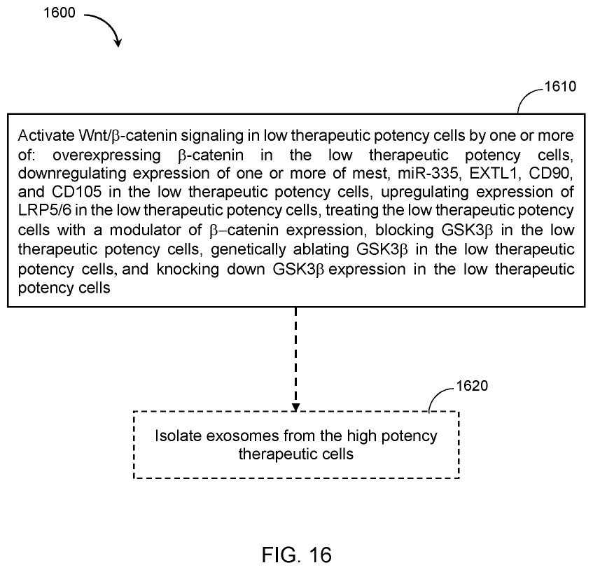

With reference to FIG. 16, an embodiment of a method of preparing high potency therapeutic cells for treating conditions requiring tissue repair, tissue regeneration, or tissue growth is described. The method 1600 can include activating 1610 Wnt/β-catenin signaling in low therapeutic potency cells by one or more of: overexpressing β-catenin in the low therapeutic potency cells, downregulating expression of one or more of mest, miR-335, EXTL1, CD90, and CD105 in the low therapeutic potency cells, upregulating expression of LRP5/6 in the low therapeutic potency cells, treating the low therapeutic potency cells with a modulator of β-catenin expression, blocking GSK3β in the low therapeutic potency cells, genetically ablating GSK3β in the low therapeutic potency cells, and knocking down GSK3β expression in the low therapeutic potency cells. Activating Wnt/β-catenin signaling in low therapeutic potency cells can generate high potency therapeutic cells having an increased therapeutic potency relative to the low therapeutic potency cells without activation of Wnt/β-catenin signaling, wherein the high potency therapeutic cells are effective for facilitating tissue repair, tissue regeneration, or tissue growth. In some embodiments, the high potency therapeutic cells find use in generating exosomes high therapeutic potency exosomes. In some embodiments, the method includes isolating 1620 exosomes (e.g., high therapeutic potency exosomes) from the high potency therapeutic cells, wherein the exosomes are effective for facilitating tissue repair, tissue regeneration, or tissue growth.

Activating Wnt/β-catenin signaling in low therapeutic potency cells can include activation by any suitable option. In some embodiments, activating Wnt/β-catenin signaling includes altering gene and/or protein expression in the low therapeutic potency cells, and/or treating the low therapeutic potency cells with a modulator of Wnt/β-catenin signaling. In some embodiments, activating Wnt/β-catenin signaling includes preferentially activating canonical Wnt signaling over non-canonical Wnt signaling in the low therapeutic potency cells. Altering gene and/or protein expression in the low therapeutic potency cells can be done using any suitable option. In some embodiments, activating Wnt/β-catenin signaling includes genetically modifying the low therapeutic potency cells to alter gene and/or protein expression. In some embodiments, activating Wnt/β-catenin signaling includes genetically modifying the low therapeutic potency cells with one or more nucleic acids encoding a mediator or modulator of canonical Wnt signaling, to thereby alter gene and/or protein expression of one or more canonical Wnt signaling pathway components, e.g., β-catenin. Any suitable option for introducing nucleic acids into the low therapeutic potency cells can be used. Suitable options for genetically modifying the low therapeutic potency cells with nucleic acids include, without limitation, transfection, transformation, viral transduction (e.g., lentiviral transduction), etc.

In some embodiments, activating Wnt/β-catenin signaling increases a level of β-catenin expression, e.g., β-catenin protein expression, in the low therapeutic potency cells by about 30%, by about 40%, by about 50%, by about 60%, by about 70%, by about 80% by about 90%, by about 100%, by about 120%, by about 140% by about 160%, by about 180%, by about 200%, by about 220%, by about 240%, by about 260%, by about 280%, by about 300% or more, or by a percentage within a range defined by any two of the preceding values.

In some embodiments, the method includes activating Wnt/β-catenin signaling by altering gene and/or protein expression in the low therapeutic potency cells. In some embodiments, activating Wnt/β-catenin signaling includes increasing gene and/or protein expression of one or more canonical Wnt signaling mediators and regulators in the low therapeutic potency cells. In some embodiments, activating Wnt/β-catenin signaling includes increasing gene and/or protein expression in the low therapeutic potency cells of one or more canonical Wnt signaling mediators and regulators that are specific to the canonical Wnt signaling pathway. In some embodiments, activating Wnt/β-catenin signaling includes increasing gene and/or protein expression of one or more canonical Wnt signaling mediators that activate the canonical Wnt signaling pathway but do not activate the non-canonical Wnt signaling pathway.

In some embodiments, the method includes overexpressing β-catenin in the low therapeutic potency cells to activate Wnt/β-catenin signaling. β-catenin can be overexpressed using any suitable option. In some embodiments, activating Wnt/β-catenin signaling includes genetically modifying the low therapeutic potency cells with a nucleic acid encoding β-catenin, where the nucleic acid is configured to express, e.g., overexpress, β-catenin in the low therapeutic potency cells. In some embodiments, β-catenin is human β-catenin (Gene ID: 1499).

In some embodiments, overexpression of β-catenin achieves an average level of β-catenin protein expression in the high potency therapeutic cells that is higher by about 30%, by about 40%, by about 50%, by about 60%, by about 70%, by about 80% by about 90%, by about 100%, by about 120%, by about 140% by about 160%, by about 180%, by about 200%, by about 220%, by about 240%, by about 260%, by about 280%, by about 300% or more, or by a percentage within a range defined by any two of the preceding values, relative to a reference population of cells, e.g., low therapeutic potency cells. The expression level of β-catenin in the high potency therapeutic cells can be compared to a suitable reference population of cells, such as the low therapeutic potency cells from which the high potency therapeutic cells were derived but in which Wnt/β-catenin signaling has not been activated, or another population of cells of the same type as the low therapeutic potency cells from which the high potency therapeutic cells were derived.

In some embodiments, activating Wnt/β-catenin signaling includes downregulating expression of one or more of mest, miR-335, EXTL1, CD90, and CD105 in the low therapeutic potency cells. In some embodiments, activating Wnt/β-catenin signaling includes downregulating mRNA and/or protein expression of one or more of mest, EXTL1, CD90, and CD105 in the low therapeutic potency cells. In some embodiments, activating Wnt/β-catenin signaling includes downregulating mRNA and/or protein expression of mest in the low therapeutic potency cells. In some embodiments, activating Wnt/β-catenin signaling includes downregulating expression of mRNA and/or protein EXTL1 in the low therapeutic potency cells. Expression of mest, miR-335, EXTL1, CD90, or CD105 can be downregulated using any suitable option. In some embodiments, downregulating expression of one or more of mest, miR-335, EXTL1, CD90, and CD105 includes using an inhibitory nucleic acid, e.g., an inhibitory RNA, such as shRNA, targeting one or more of mest, miR-335, EXTL1, CD90, or CD105, respectively. In some embodiments, downregulating expression of one or more of mest, miR-335, EXTL1, CD90, and CD105 includes genetically modifying low therapeutic potency cells with a nucleic acid encoding an inhibitory nucleic acid, e.g., an inhibitory RNA, such as shRNA, targeting one or more of mest, miR-335, EXTL1, CD90, or CD105, respectively, and configured to express the inhibitory nucleic acid in the low therapeutic potency cells. In some embodiments, downregulating expression of one or more of mest, miR-335, EXTL1, CD90, and CD105 includes treating the low therapeutic potency cells with an agent that reduces expression of one or more of mest, miR-335, EXTL1, CD90, and CD105, respectively. The agent that reduces expression of one or more of mest, miR-335, EXTL1, CD90, and CD105 can be any suitable compound. In some embodiments, an agent that reduces expression of one or more of mest, miR-335, EXTL1, CD90, and CD105 is, without limitation, tideglusib or 6-bromoindirubin-3′-oxime (BIO). In some embodiments, downregulating expression of one or more of mest, miR-335, EXTL1, CD90, and CD105 includes genetically modifying low therapeutic potency cells to overexpress β-catenin and/or gata4.

In some embodiments, activating Wnt/β-catenin signaling includes downregulating expression of one or more of mest, miR-335, EXTL1, CD90, and CD105 in the low therapeutic potency cells by about 2 fold, about 3 fold, about 4 fold, about 5 fold, about 6 fold, about 8 fold, about 10 fold, about 15 fold, about 20 fold, about 25 fold, about 30 fold, about 35 fold, about 40 fold or more, or by a fold amount within a range defined by any two of the preceding values.

In some embodiments, activating Wnt/β-catenin signaling includes upregulating expression of LRP5/6 in the low therapeutic potency cells. In some embodiments, activating Wnt/β-catenin signaling includes upregulating protein expression of LRP5/6 in the low therapeutic potency cells. In some embodiments, activating Wnt/β-catenin signaling includes upregulating protein expression of LRP5/6 in the low therapeutic potency cells. In some embodiments, activating Wnt/β-catenin signaling includes upregulating cell surface expression of LRP5/6 in the low therapeutic potency cells. In some embodiments, activating Wnt/β-catenin signaling does not include upregulating mRNA expression of lrp5 or lrp6 in the low therapeutic potency cells. Upregulating expression of LRP5/6 in the low therapeutic potency cells can be achieved using any suitable option. In some embodiments, upregulating expression of LRP5/6 in the low therapeutic potency cells includes treating the low therapeutic potency cells with an agent that increases expression of LRP5/6. In some embodiments, an agent that increases expression of LRP5/6 is, without limitation, tideglusib or 6-bromoindirubin-3′-oxime (BIO). In some embodiments, upregulating expression of LRP5/6 includes genetically modifying low therapeutic potency cells to overexpress β-catenin and/or gata4. In some embodiments, upregulating expression of LRP5/6 in the low therapeutic potency cells includes using an inhibitory nucleic acid, e.g., an inhibitory RNA, such as shRNA, targeting mest.

In some embodiments, activating Wnt/β-catenin signaling includes upregulating cell surface expression of LRP5/6 in the low therapeutic potency cells such that the fraction of cells expressing LRP5/6, e.g., as determined by flow cytometry, is increased by about 10%, by about 15%, by about 20%, by about 25%, by about 30%, by about 35%, by about 40%, by about 45%, by about 50%, by about 55%, by about 60%, by about 65%, by about 70%, by about 75%, by about 80%, or more, or by a percentage within a range defined by any two of the preceding values.

In some embodiments, activating Wnt/β-catenin signaling includes treating the low therapeutic potency cells with a modulator of β-catenin expression. The modulator of β-catenin expression can be any suitable agent that activates Wnt/β-catenin signaling. In some embodiments, the modulator of β-catenin expression increases β-catenin expression, e.g., β-catenin protein expression. In some embodiments, the modulator of β-catenin expression is, without limitation, tideglusib or 6-bromoindirubin-3′-oxime (BIO). In some embodiments, the method includes contacting the low therapeutic potency cells with the modulator of β-catenin expression to activate Wnt/β-catenin signaling. In some embodiments, the low therapeutic potency cells are treated with an effective amount of the modulator of β-catenin expression for about 12 hours, about 16 hours, about 20 hours, about 24 hours, about 28 hours, about 32 hours, about 36 hours, about 40 hours, about 44 hours, about 48 hours, about 54 hours, about 60 hours, about 66 hours, about 72 hours or more, or for a time interval within a range defined by any two of the preceding values.

In some embodiments, activating Wnt/β-catenin signaling includes blocking GSK3β in the low therapeutic potency cells. Any suitable option can be used to block GSK3β in the low therapeutic potency cells. In some embodiments, the method includes treating the low therapeutic potency cells with a modulator of β-catenin expression, e.g., tideglusib or 6-bromoindirubin-3′-oxime (BIO), to thereby block GSK3β in the low therapeutic potency cells. In some embodiments, the method includes downregulating expression of mest to thereby block GSK3β in the low therapeutic potency cells, as described herein. In some embodiments, downregulating expression of mest includes genetically modifying the low therapeutic potency cells with an inhibitory nucleic acid, e.g., an inhibitory RNA, such as shRNA, targeting mest, to thereby block GSK3β in the low therapeutic potency cells. In some embodiments, downregulating expression of mest includes treating the low therapeutic potency cells with a modulator of β-catenin expression, e.g., tideglusib or 6-bromoindirubin-3′-oxime (BIO), to thereby block GSK3β in the low therapeutic potency cells.

In some embodiments, the low therapeutic potency cells are treated with about 0.1 μM, about 0.2 μM, about 0.5 μM, about 1 μM, about 1.5 μM, about 2 μM, about 2.5 μM, about 3 μM, about 3.5 μM, about 4 μM, about 4.5 μM, about 5 μM, about, 5.5 μM, about 6 μM, about 6.5 μM, about 7 μM, about 8 μM, about 9 μM, about 10 μM, about 11 μM, about 12 μM, about 13 μM, about 14 μM, about 15 μM or more, or a concentration within a range defined by any two of the preceding values, of BIO to activate Wnt/β-catenin signaling. In some embodiments, the low therapeutic potency cells are treated with about 0.1 μM, about 0.2 μM, about 0.5 μM, about 1 μM, about 1.5 μM, about 2 μM, about 2.5 μM, about 3 μM, about 3.5 μM, about 4 μM, about 4.5 μM, about 5 μM, about, 5.5 μM, about 6 μM, about 6.5 μM, about 7 μM, about 8 μM, about 9 μM, about 10 μM, about 11 μM, about 12 μM, about 13 μM, about 14 μM, about 15 μM or more, or a concentration within a range defined by any two of the preceding values, of tideglusib to activate Wnt/β-catenin signaling.

The low therapeutic potency cells can be any suitable type of cell having low therapeutic potency. In some embodiments, the low therapeutic potency cells are mammalian cells. In some embodiments, the low therapeutic potency cells are human cells. In some embodiments, the low therapeutic potency cells are primary cells. In some embodiments, the low therapeutic potency cells are a cell line. In some embodiments, the low therapeutic potency cells are immortalized cells. In some embodiments, the low therapeutic potency cells are genetically modified cells, e.g., cells genetically modified to overexpress gata4.

In some embodiments, the low therapeutic potency cells are fibroblast cells, e.g., normal human dermal fibroblasts (NHDF). In some embodiments, the fibroblast cells are genetically modified to overexpress gata4. In some embodiments, the fibroblast cells express gata4 mRNA at a level that is higher than the expression level in fibroblast cells that do not overexpress gata4 by a log 2 fold of about 0.2, about 0.3, about 0.4, about 0.5, about 0.7, about 1, about 1.5, about 2, about 2.5, about 3, about 3.5, about 4 or more, or higher by a log₂ fold within a range defined by any two of the preceding values. In some embodiments, the method includes genetically modifying the fibroblast cells to overexpress gata4. The fibroblast cells can be genetically modified using any suitable option. In some embodiments, genetically modifying the fibroblast cells includes introducing a nucleic acid encoding gata4 into the fibroblast cells by transduction, e.g., viral transduction, such as lentiviral transduction.

In some embodiments, the low therapeutic potency cells are low therapeutic potency cardiosphere-derived cells (CDCs). In some embodiments, the low therapeutic potency CDCs are from a line of CDCs, e.g., from the same donor, that produce low therapeutic potency CDCs. In some embodiments, the low therapeutic potency CDCs are from a line of CDCs, e.g., CDCs from the same donor, that produces CDCs having lot-to-lot variation in therapeutic potency. In some embodiments, the low therapeutic potency CDCs are immortalized CDCs.

In some embodiments, the method includes immortalizing CDCs to generate the immortalized CDCs. The CDCs may be immortalized using any suitable option. In some embodiments, the CDCs are immortalized using simian virus 40 large and small antigens (SV40 T+t). In some embodiments, the CDCs are immortalized using HPV E6 and E7, Epstein-Barr virus, hTERT, or fusion with an immortalized cell line. In some embodiments, the CDCs are high therapeutic potency CDCs before immortalization. In some embodiments, the CDCs have variable therapeutic potency, e.g., where some lots of CDCs have high therapeutic potency, and other lots obtained from the same donor have low therapeutic potency, before immortalization.

In some embodiments, the method includes determining a population of cells as having low therapeutic potency. In some embodiments, determining comprises measuring an expression level, e.g., protein or mRNA level, of one or more Wnt/β-catenin signaling mediators and regulators in the population of cells. In some embodiments, the one or more Wnt/β-catenin signaling mediators and regulators are specific to canonical Wnt/β-catenin signaling. In some embodiments, the one or more Wnt/β-catenin signaling mediators and regulators is selected from: β-catenin, LRP5/6, mest, and EXTL1. In some embodiments, the cells are determined to have low therapeutic potency based on a comparison of the measured expression level of the one or more Wnt/β-catenin signaling mediators and regulators with a reference level or reference range, e.g., expression level or range of the corresponding Wnt/β-catenin signaling mediator or regulator in high and/or low therapeutic potency cells. In some embodiments, the cells are determined to have low therapeutic potency if the measured expression level of β-catenin and/or LRP5/6 is below a reference level, e.g., a corresponding level of expression of the one or more Wnt/β-catenin signaling mediators and regulators in high potency therapeutic cells. In some embodiments, the cells are determined to have low therapeutic potency if the measured expression level of mest and/or EXTL1 is above a reference level, e.g., a corresponding level of expression of the one or more Wnt/β-catenin signaling mediators and regulators in high potency therapeutic cells.

In some embodiments, determining comprises measuring an mRNA level of one or more non-canonical Wnt signaling mediators. In some embodiments, the therapeutic potency of the cells are determined based on the measured expression level of one or more Wnt/β-catenin signaling mediators and regulators, and the measured mRNA level of the one or more non-canonical Wnt signaling mediators, in the population of cells. The measured mRNA level of the one or more non-canonical Wnt signaling mediators can be compared to a suitable reference mRNA level or range, e.g., an mRNA level or range in high and/or low therapeutic potency cells. In some embodiments, the one or more non-canonical Wnt signaling mediators is selected from: ror2, nfatc2, axin2, rac2, and apcdd1.

Also provided herein is a method of determining whether a population of cells has a high therapeutic potency or low therapeutic potency, by measuring an expression level of one or more Wnt/β-catenin signaling mediators and regulators in the population of cells; and determining the population of cells as having high therapeutic potency or low therapeutic potency based on the measured level of the one or more Wnt/β-catenin signaling mediators and regulators. In some embodiments, the method includes comparing the measured level of the one or more Wnt/β-catenin signaling mediators and regulators to a reference level or reference range. In some embodiments, the reference level is based on the level the one or more Wnt/β-catenin signaling mediators and regulators in a population of cells having low therapeutic potency. In some embodiments, the reference level is based on the level the one or more Wnt/β-catenin signaling mediators and regulators in a population of cells having high therapeutic potency. In some embodiments, the reference range is the range of levels of the one or more Wnt/β-catenin signaling mediators and regulators in a population of cells having low or high therapeutic potency. In some embodiments, the population of cells is derived from a source of cells having variable therapeutic potency. In some embodiments, the population of cells comprises fibroblasts or CDCs. In some embodiments, the one or more Wnt/β-catenin signaling mediators and regulators includes, without limitation, one or more of β-catenin, LRP5/6, mest, and EXTL1. In some embodiments, the population of cells are determined to have high therapeutic potency upon determining the measured level of β-catenin and/or LRP5/6 is above a reference level (e.g., the reference level for low therapeutic potency cells), and/or within a reference range (e.g., a reference range for high potency therapeutic cells). In some embodiments, the method includes measuring an mRNA level of one or more non-canonical Wnt signaling mediators. In some embodiments, the method includes determining the population of cells as having high therapeutic potency or low therapeutic potency based on the measured level of the one or more Wnt/β-catenin signaling mediators and regulators, and the measured level of the one or more non-canonical Wnt signaling mediators. The measured mRNA level of the one or more non-canonical Wnt signaling mediators can be compared to a suitable reference mRNA level or range, e.g., an mRNA level or range in high and/or low therapeutic potency cells. In some embodiments, the one or more non-canonical Wnt signaling mediators is selected from: ror2, nfatc2, axin2, rac2, and apcdd1.

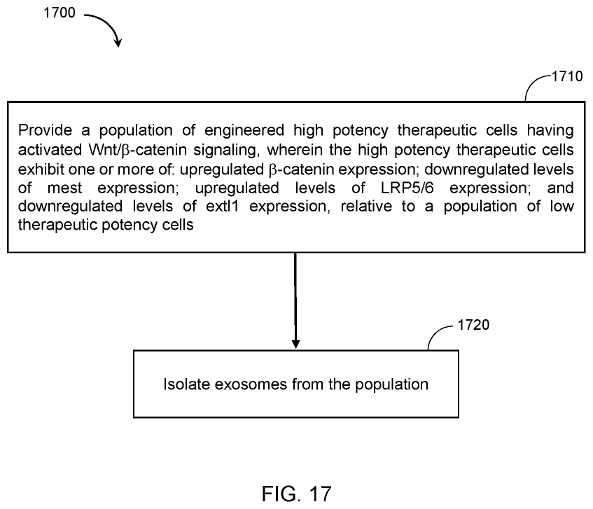

With reference to FIG. 17, an embodiment of a method of preparing high therapeutic potency exosomes for treating conditions requiring tissue repair, tissue regeneration, or tissue growth is described. The method 1700 can include providing 1710 a population of engineered high potency therapeutic cells having activated Wnt/β-catenin signaling, wherein the high potency therapeutic cells exhibit one or more of upregulated β-catenin expression; downregulated levels of mest expression; upregulated levels of LRP5/6 expression; and downregulated levels of extl1 expression, relative to a population of low therapeutic potency cells. The method can include isolating 1720 exosomes from the population. The exosomes can be isolated from the population of engineered high potency therapeutic cells using any suitable option, as described herein. The isolated exosomes can have an increased therapeutic potency relative to low therapeutic potency exosomes isolated from the low therapeutic potency cells without the activated Wnt/β-catenin signaling, wherein the high therapeutic potency exosomes are effective for facilitating tissue repair, tissue regeneration, or tissue growth.

In some embodiments, the high potency therapeutic cells exhibit upregulated β-catenin expression. In some embodiments, the high potency therapeutic cells have a level of β-catenin expression, e.g., β-catenin protein expression, that is higher than low therapeutic potency cells by about 30%, by about 40%, by about 50%, by about 60%, by about 70%, by about 80% by about 90%, by about 100%, by about 120%, by about 140% by about 160%, by about 180%, by about 200%, by about 220%, by about 240%, by about 260%, by about 280%, by about 300% or more, or by a percentage within a range defined by any two of the preceding values. In some embodiments, the high potency therapeutic cells exhibit upregulated LRP5/6 expression. In some embodiments, the high potency therapeutic cells have a level of LRP5/6 expression, e.g., LRP5/6 cell surface expression, that is higher than low therapeutic potency cells by about 10%, by about 15%, by about 20%, by about 25%, by about 30%, by about 35%, by about 40%, by about 45%, by about 50%, by about 55%, by about 60%, by about 65%, by about 70%, by about 75%, by about 80%, or more, or by a percentage within a range defined by any two of the preceding values.

In some embodiments, the high potency therapeutic cells exhibit downregulated levels of mest expression. In some embodiments, the high potency therapeutic cells have a level of mest expression that is lower than low therapeutic potency cells by about 2 fold, about 3 fold, about 4 fold, about 5 fold, about 6 fold, about 8 fold, about 10 fold, about 15 fold, about 20 fold, about 25 fold, about 30 fold or more, or by a fold amount within a range defined by any two of the preceding values. In some embodiments, the high potency therapeutic cells exhibit downregulated levels of extl1 expression. In some embodiments, the high potency therapeutic cells have a level of extl1 expression that is lower than low therapeutic potency cells by about 2 fold, about 3 fold, about 4 fold, about 5 fold, about 6 fold, about 8 fold, about 10 fold, about 15 fold, about 20 fold, about 25 fold, about 30 fold or more, or by a fold amount within a range defined by any two of the preceding values.

In some embodiments, providing the population of engineered high potency therapeutic cells includes preparing high potency therapeutic cells for treating conditions requiring tissue repair, tissue regeneration, or tissue growth according to any method as disclosed herein. In some embodiments, providing the population of engineered high potency therapeutic cells includes identifying low therapeutic potency cells; and activating Wnt/β-catenin signaling in the low therapeutic potency cells by one or more of: overexpressing β-catenin in the low therapeutic potency cells, downregulating expression of one or more of mest, miR-335, EXTL1, CD90, and CD105 in the low therapeutic potency cells, upregulating expression of LRP5/6 in the low therapeutic potency cells, treating the low therapeutic potency cells with a modulator of β-catenin expression, and blocking GSK3β in the low therapeutic potency cells, to thereby generate a population of cells enriched in the engineered high potency therapeutic cells.

In some embodiments, the high therapeutic potency exosomes comprise increased levels of miR-92a, increased levels of miR-146a, decreased levels of miR-199b, or combinations thereof. In some embodiments, the high therapeutic potency exosomes comprise increased levels of miR-92a relative to a suitable reference level or reference range, increased levels of miR-146a relative to a suitable reference level or reference range, and/or decreased levels of miR-199b relative to a suitable reference level or reference range. The reference level or reference range can be, in some embodiments, a level or range of the corresponding miRNA in low therapeutic potency exosomes.

In some embodiments, the high therapeutic potency exosomes comprise increased levels of miR-92a relative to low therapeutic potency exosomes. In some embodiments, the amount of miR-92a in the high therapeutic potency exosomes is higher than the amount in low therapeutic potency exosomes by a log₂ fold of about 1, about 1.2, about 1.5, about 2, about 2.2, about 2.5, about 3, about 3.2, about 3.5, about 4, about 4.2, about 4.5, about 5 or more, or higher by a log 2 fold within a range defined by any two of the preceding values. In some embodiments, the high therapeutic potency exosomes comprise increased levels of miR-146a relative to low therapeutic potency exosomes. In some embodiments, the amount of miR-146a in the high therapeutic potency exosomes is higher than the amount in low therapeutic potency exosomes by a log 2 fold of about 1, about 1.2, about 1.5, about 2, about 2.2, about 2.5, about 3, about 3.2, about 3.5, about 4, about 4.2, about 4.5, about 5 or more, about 5.2, about 5.5, about 6, about 6.2, about 6.5, about 7, about 7.2, about 7.5, about 8, about 8.2, about 8.5, about 9, about 9.2, about 9.5, about 10 or more, or higher by a log 2 fold within a range defined by any two of the preceding values. In some embodiments, the high therapeutic potency exosomes comprise decreased levels of miR-199b relative to low therapeutic potency exosomes. In some embodiments, the amount of miR-199b in the high therapeutic potency exosomes is lower than the amount in low therapeutic potency exosomes by a log₂ fold of about 1, about 1.2, about 1.5, about 2, about 2.2, about 2.5, about 3, about 3.2, about 3.5, about 4, about 4.2, about 4.5, about 5 or more, or lower by a log 2 fold within a range defined by any two of the preceding values.

In some embodiments, the high therapeutic potency exosomes comprise one or more exosomal surface markers. In some embodiments, exosomal surface markers are selected from one or more of: ITGB1, HSC70, CD9, CD63, and GAPDH. In some embodiments, high therapeutic potency exosomes derived from CDCs, e.g., immortalized CDCs, are enriched with respect to expression of one or more of ITGB1, HSC70, CD63, and GAPDH (e.g., as compared to low potency exosomes). In some embodiments, high therapeutic potency exosomes derived from CDCs, e.g., immortalized CDCs, are enriched with respect to expression of one or more of ITGB1, HSC70, and GAPDH. In some embodiments, high therapeutic potency exosomes derived from CDCs, e.g., immortalized CDCs, do not express CD9. In some embodiments, high therapeutic potency exosomes derived from CDCs, e.g., immortalized CDCs, are depleted for expression of CD9 (e.g., as compared to low potency exosomes). In some embodiments, high therapeutic potency exosomes derived from CDCs, e.g., immortalized CDCs, are enriched for expression of one or more of ITGB1, HSC70, and GAPDH, and are depleted for CD9 expression. In some embodiments, high therapeutic potency exosomes derived from engineered fibroblasts are enriched with respect to expression of one or more of ITGB1, CD9, and CD63. In some embodiments, high therapeutic potency exosomes derived from engineered fibroblasts are depleted for HSC70 expression and/or GAPDH expression. In some embodiments, high therapeutic potency exosomes derived from engineered fibroblasts are enriched for expression of one or more of ITGB1, CD9, and CD63, and are depleted for HSC70 expression and/or GAPDH expression.

The high potency therapeutic cells and/or high therapeutic potency exosomes can be prepared from any suitable source of cells. In some embodiments, the low therapeutic potency cells are allogeneic to a subject in need of treating a condition requiring the tissue repair, tissue regeneration, or tissue growth (e.g., by administering to the subject an effective amount of high potency therapeutic cells and/or high therapeutic potency exosomes). In some embodiments, the low therapeutic potency cells are autologous to a subject in need of treating a condition requiring the tissue repair, tissue regeneration, or tissue growth (e.g., by administering to the subject an effective amount of high potency therapeutic cells and/or high therapeutic potency exosomes). In some embodiments, the low therapeutic potency cells are heterologous to a subject in need of treating a condition requiring the tissue repair, tissue regeneration, or tissue growth (e.g., by administering to the subject an effective amount of high potency therapeutic cells and/or high therapeutic potency exosomes).

In some embodiments, high potency therapeutic exosomes are prepared from low therapeutic potency cells that are allogeneic to a subject in need of treating a condition requiring the tissue repair, tissue regeneration, or tissue growth (e.g., by administering to the subject an effective amount of high therapeutic potency exosomes). In some embodiments, high potency therapeutic exosomes are prepared from low therapeutic potency cells that are autologous to a subject in need of treating a condition requiring the tissue repair, tissue regeneration, or tissue growth (e.g., by administering to the subject an effective amount of high therapeutic potency exosomes). In some embodiments, high potency therapeutic exosomes are prepared from low therapeutic potency cells that are heterologous to a subject in need of treating a condition requiring the tissue repair, tissue regeneration, or tissue growth (e.g., by administering to the subject an effective amount of high therapeutic potency exosomes).

The conditions requiring tissue repair, tissue regeneration, or tissue growth can vary. In some embodiments, the conditions requiring tissue repair, tissue regeneration, or tissue growth include, without limitation, one or more of muscular disorders, myocardial infarction, cardiac disorders, myocardial alterations, muscular dystrophy, fibrotic disease, inflammatory disease, and wound healing. The therapeutic potency of cells and/or exosomes, according to some embodiments, can include a variety of therapeutic effects that are desired to treat a subject in need of treatment of the condition. In general, conditions that can be treated by the therapeutic cells and/or exosomes include, without limitation, one or more of muscular disorders, myocardial infarction, cardiac disorders, myocardial alterations, muscular dystrophy, fibrotic disease, inflammatory disease, and wound healing. In some embodiments, the condition is a muscular disorder, e.g., muscular dystrophy. In some embodiments, the condition is myocardial infarction.

In some embodiments, high potency therapeutic cells and/or high therapeutic potency exosomes of the present disclosure are effective for one or more of reducing cardiac scar size, increasing myocardial infarct wall thickness, increasing ejection fraction, reducing mortality from myocardial infarction, increasing exercise capacity, reducing skeletal muscle fibrosis, and increasing myofiber size, when administered to a subject in need of treating a condition requiring tissue repair, tissue regeneration, or tissue growth. In some embodiments, the increased therapeutic potency comprises a difference in a percentage therapeutic effect between the high potency therapeutic cells and the low potency therapeutic cells of about 5%, about 10%, about 15%, about 20%, about 25%, about 30%, about 35%, about 40%, or more, or a difference in percentage within a range defined by any two of the preceding values. In some embodiments, the increased therapeutic potency comprises a difference in a percentage therapeutic effect between the high therapeutic potency exosomes and exosomes prepared from low therapeutic potency cells, e.g., low therapeutic potency exosomes, of about 5%, about 10%, about 15%, about 20%, about 25%, about 30%, about 35%, about 40%, or more, or a difference in percentage within a range defined by any two of the preceding values.

In some embodiments, low therapeutic potency cells have substantially no therapeutic effect. In some embodiment, low therapeutic potency cells have substantially no effect on reducing cardiac scar size, increasing myocardial infarct wall thickness, increasing ejection fraction, reducing mortality from myocardial infarction, increasing exercise capacity, reducing skeletal muscle fibrosis, and increasing myofiber size, when administered to a subject in need of treating a condition requiring tissue repair, tissue regeneration, or tissue growth.

### Treatment Modalities for Damaged or Diseased Tissues

Generally, the use of one or more relatively common therapeutic modalities are used to treat damaged or diseased tissues in an effort to halt progression of the disease, reverse damage that has already occurred, prevent additional damage, and generally improve the well-being of the patient. For example, many conditions can be readily treated with holistic methodologies or changes in lifestyle (e.g., improved diet to reduce risk of cardiovascular disease, diabetes, and the like). Often more serious conditions require more advanced medical intervention. Drug therapy or pharmaceutical therapies are routinely administered to treat patients suffering from a particular disease. For example, a patient suffering from high blood pressure might be prescribed an angiotensin-converting-enzyme (ACE) inhibitor, in order to reduce the tension of blood vessels and blood volume, thereby treating high blood pressure. Further, cancer patients are often prescribed panels of various anticancer compounds in an attempt to limit the spread and/or eradicate a cancerous tumor. Surgical methods may also be employed to treat certain diseases or injuries. In some cases, implanted devices are used in addition to or in place of pharmaceutical or surgical therapies (e.g., a cardiac pacemaker). Recently, additional therapy types have become very promising, such as, for example, gene therapy, protein therapy, and cellular therapy.

Cell therapy, generally speaking, involves the administration of population of cells to subject with the intent of the administered cells functionally or physically replacing cells that have been damaged, either by injury, by disease, or combinations thereof. A variety of different cell types can be administered in cell therapy, with stem cells being particularly favored (in certain cases) due to their ability to differentiate into multiple cell types, thus providing flexibility for what disease or injury they could be used to treat.

Protein therapy involves the administration of exogenous proteins that functionally replace deficient proteins in the subject suffering from a disease or injury. For example, synthesized acid alpha-glucosidase is administered to patients suffering from glycogen storage disease type II.

In addition, nucleic acid therapy is being investigated as a possible treatment for certain diseases or conditions. Nucleic acid therapy involves the administration of exogenous nucleic acids, or short fragments thereof, to the subject in order to alter gene expression pathways through a variety of mechanisms, such as, for example, translational repression of the target gene, cleavage of a target gene, such that the target gene product is never expressed.

With the knowledge that certain cellular therapies provide profound regenerative effects, several embodiments disclosed herein involve methods and compositions that produce those regenerative effects without the need for administration of cells to a subject (though cells may optionally be administered in certain embodiments). Several embodiments disclosed herein provide for the generation of high therapeutic potency cells and exosomes.

### Exosomes and Vesicle Bound Nucleic Acid and Protein Products

Nucleic acids are generally not present in the body as free nucleic acids, as they are quickly degraded by nucleases. Certain types of nucleic acids are associated with membrane-bound particles. Such membrane-bound particles are shed from most cell types and consist of fragments of plasma membrane and contain DNA, RNA, mRNA, microRNA, and proteins. These particles often mirror the composition of the cell from which they are shed. Exosomes are one type of such membrane bound particles and typically range in diameter from about 15 nm to about 95 nm in diameter, including about 15 nm to about 20 nm, 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm, and overlapping ranges thereof. In several embodiments, exosomes are larger (e.g., those ranging from about 140 to about 210 run, including about 140 nm to about 150 nm, 150 nm to about 160 run, 160 nm to about 170 run, 170 nm to about 180 nm, 180 nm to about 190 run, 190 nm to about 200 run, 200 nm to about 210 nm, and overlapping ranges thereof). In some embodiments, the exosomes that are generated from the original cellular body are 100, 200, 300, 400, 500, 600, 700, 800, 900, 1000, 2000, 5000, 10,000 times smaller in at least one dimension (e.g., diameter) than the original cellular body.

Alternative nomenclature is also often used to refer to exosomes. Thus, as used herein the term “exosome” shall be given its ordinary meaning and may also include terms including microvesicles, epididimosomes, argosomes, exosome-like vesicles, microparticles, promininosomes, prostasomes, dexosomes, texosomes, dex, tex, archeosomes and oncosomes. Unless otherwise indicated herein, each of the foregoing terms shall also be understood to include engineered high-potency varieties of each type of exosome. Exosomes are secreted by a wide range of mammalian cells and are secreted under both normal and pathological conditions. Exosomes, in some embodiments, function as intracellular messengers by virtue of carrying mRNA, miRNA or other contents from a first cell to another cell (or plurality of cells). In several embodiments, exosomes are involved in blood coagulation, immune modulation, metabolic regulation, cell division, and other cellular processes. Because of the wide variety of cells that secret exosomes, in several embodiments, exosome preparations can be used as a diagnostic tool (e.g., exosomes can be isolated from a particular tissue, evaluated for their nucleic acid or protein content, which can then be correlated to disease state or risk of developing a disease).

Exosomes, in several embodiments, are isolated from cellular preparations by methods comprising one or more of filtration, centrifugation, antigen-based capture and the like. For example, in several embodiments, a population of cells grown in culture are collected and pooled. In several embodiments, monolayers of cells are used, in which case the cells are optionally treated in advance of pooling to improve cellular yield (e.g., dishes are scraped and/or enzymatically treated with an enzyme such as trypsin to liberate cells). In some embodiments, cells grown in culture under standard cell culture conditions are exposed to serum-free medium under hypoxic condition overnight, and conditioned media containing exosomes are collected. In some embodiments, the hypoxic condition includes about 15%, about 12%, about 10%, about 9%, about 8%, about 7%, about 6%, about 5%, about 4%, about 3%, about 2%, about 1%, O₂ or less, or a percentage of O₂ in a range defined by any two of the preceding values. In some embodiments, the hypoxic condition includes 2% 02/5% CO₂ at 37 °C. In some embodiments, the cells exposed to hypoxic condition recover in complete serum under standard culture conditions for about 24, about 36, about 48, about 60, about 72 hours or more, or a time interval in a range defined by any two of the preceding values, and are then re-exposed to hypoxic condition to generate condition media. In some embodiments, cells are cycled between hypoxic and standard cell culture conditions for 1, 2, 3, 4, 5, 6 or more times. In several embodiments, cells grown in suspension are used. The pooled population is then subject to one or more rounds of centrifugation (in several embodiments ultracentrifugation and/or density centrifugation is employed) in order to separate the exosome fraction from the remainder of the cellular contents and debris from the population of cells. In some embodiments, centrifugation need not be performed to harvest exosomes. In several embodiments, pre-treatment of the cells is used to improve the efficiency of exosome capture. For example, in several embodiments, agents that increase the rate of exosome secretion from cells are used to improve the overall yield of exosomes. In some embodiments, augmentation of exosome secretion is not performed. In some embodiments, size exclusion filtration is used in conjunction with, or in place of centrifugation, in order to collect a particular size (e.g., diameter) of exosome. In several embodiments, filtration need not be used. In still additional embodiments, exosomes (or subpopulations of exosomes are captured by selective identification of unique markers on or in the exosomes (e.g., transmembrane proteins)). In such embodiments, the unique markers can be used to selectively enrich a particular exosome population. In some embodiments, enrichment, selection, or filtration based on a particular marker or characteristic of exosomes is not performed.

Upon administration (discussed in more detail below) exosomes can fuse with the cells of a target tissue. As used herein, the term “fuse” shall be given its ordinary meaning and shall also refer to complete or partial joining, merging, integration, or assimilation of the exosome and a target cell. In several embodiments, the exosomes fuse with healthy cells of a target tissue. In some embodiments, the fusion with healthy cells results in alterations in the healthy cells that leads to beneficial effects on the damaged or diseased cells (e.g., alterations in the cellular or intercellular environment around the damaged or diseased cells). In some embodiments, the exosomes fuse with damaged or diseased cells. In some such embodiments, there is a direct effect on the activity, metabolism, viability, or function of the damaged or diseased cells that results in an overall beneficial effect on the tissue. In several embodiments, fusion of the exosomes with either healthy or damaged cells is not necessary for beneficial effects to the tissue as a whole (e.g., in some embodiments, the exosomes affect the intercellular environment around the cells of the target tissue). Thus, in several embodiments, fusion of the exosome to another cell does not occur. In several embodiments, there is no cell-exosome contact, yet the exosomes still influence the recipient cells.

### Administration and Therapy

There are provided herein methods and compositions for use in the repair or regeneration of cells or tissue after the cells or tissue have been subject to injury, damage, disease, or some other event that leads to loss of function and/or viability. Methods and compositions for preventing damage and/or for shuttling nucleic acids (or proteins) between cells are also provided, regardless of whether tissue damage is present.

In addition, methods are provided for facilitating the generation of exosomes, and in particular exosomes engineered for high potency. In several such embodiments, a hydrolase is used to facilitate the liberation (e.g., secretion) of exosomes from cells. In certain embodiments, hydrolases that cleave one or more of ester bonds, sugars (e.g., DNA), ether bonds, peptide bonds, carbon-nitrogen bonds, acid anhyrides, carbon-carbon bonds, halide bonds, phosphorous-nitrogen bonds, sulpher-nitrogen bonds, carbon-phosphorous bonds, sulfur-sulfur bonds, and/or carbon-sulfur bonds are used. In some embodiments, the hydrolases are DNAses (e.g., cleave sugars). Certain embodiments employ specific hydrolases, such as for example, one or more of lysosomal acid sphingomyelinase, secreted zinc-dependent acid sphingomyelinase, neutral sphingomyelinase, and alkaline sphingomyelinase.

In several embodiments, exosomes are administered to a subject in order to initiate the repair or regeneration of cells or tissue. In several embodiments, the exosomes are derived from a stem cell. In several embodiments, the stem cells are non-embryonic stem cells. In some embodiments, the non-embryonic stem cells are adult stem cells. However, in certain embodiments, embryonic stem cells are optionally used as a source for exosomes. In some embodiments, somatic cells (by way of non-limiting example, fibroblasts) are used as a source for exosomes. In still additional embodiments, germ cells are used as a source for exosomes.

In some embodiments, cells with high therapeutic potency are generated, as described herein. In some embodiments, cells are engineered to produce exosomes of high therapeutic potency. Any cell type can be used to generate cells with high therapeutic potency and/or that produce exosomes of high therapeutic potency. For example, cardioshpere derived cells (CDCs) or fibroblast cells can be used.

In several embodiments employing stem cells as an exosome source, the nucleic acid and/or protein content of exosomes from stem cells are particularly suited to effect the repair or regeneration of damaged or diseased cells. In several embodiments, exosomes are isolated from stem cells derived from the tissue to be treated. For example, in some embodiments where cardiac tissue is to be repaired, exosomes are derived from cardiac stem cells. Cardiac stem cells are obtained, in several embodiments, from various regions of the heart, including but not limited to the atria, septum, ventricles, auricola, and combinations thereof (e.g., a partial or whole heart may be used to obtain cardiac stem cells in some embodiments). In several embodiments, exosomes are derived from cells (or groups of cells) that comprise cardiac stem cells or can be manipulated in culture to give rise to cardiac stem cells (e.g., cardiospheres and/or cardiosphere derived cells (CDCs)). Further information regarding the isolation of cardiospheres can be found in U.S. Pat. No. 8,268,619, issued on Sep. 18, 2012, which is incorporated in its entirety by reference herein. In several embodiments, the cardiac stem cells are cardiosphere-derived cells (CDCs). Further information regarding methods for the isolation of CDCs can be found in U.S. patent application Ser. No. 11/666,685, filed on Apr. 21, 2008, and Ser. No. 13/412,051, filed on Mar. 5, 2012, both of which are incorporated in their entirety by reference herein. Other varieties of stem cells may also be used, depending on the embodiment, including but not limited to bone marrow stem cells, adipose tissue derived stem cells, mesenchymal stem cells, induced pluripotent stem cells, hematopoietic stem cells, and neuronal stem cells.

In several embodiments, administration of exosomes is particularly advantageous because there are reduced complications due to immune rejection by the recipient. Certain types of cellular or gene therapies are hampered by the possible immune response of a recipient of the therapy. As with organ transplants or tissue grafts, certain types of foreign cells (e.g., not from the recipient) are attacked and eliminated (or rendered partially or completely non-functional) by recipient immune function. One approach to overcome this is to co-administer immunosuppressive therapy, however this can be costly, and leads to a patient being subject to other infectious agents. Thus, exosomal therapy is particularly beneficial because the immune response is limited. In several embodiments, this allows the use of exosomes derived from allogeneic cell sources (though in several embodiments, autologous sources are used). Moreover, the reduced potential for immune response allows exosomal therapy to be employed in a wider patient population, including those that are immune-compromised and those that have hyperactive immune systems. Moreover, in several embodiments, because the exosomes do not carry a full complement of genetic material, there is a reduced risk of unwanted cellular growth (e.g., teratoma formation) post-administration. In several embodiments, in order to further reduce the risk of recipient immune response and/or teratoma formation, exosomes (e.g., exosomes engineered for high potency), can be further manipulated, for example through gene editing using, for example CRISPR-Cas, zinc finger nucleases, and/or TALENs, to reduce their potential immunogenicity. Advantageously, the exosomes can be derived, depending on the embodiment, from cells obtained from a source that is allogeneic, autologous, xenogeneic, or syngeneic with respect to the eventual recipient of the exosomes. Moreover, master banks of exosomes that have been characterized for their expression of certain miRNAs and/or proteins can be generated and stored long-term for subsequent use in defined subjects on an “off-the-shelf” basis. However, in several embodiments, exosomes are isolated and then used without long-term or short-term storage (e.g., they are used as soon as practicable after their generation).

In several embodiments, exosomes need not be administered; rather the nucleic acid and/or protein carried by exosomes can be administered to a subject in need of tissue repair. In such embodiments, exosomes are harvested as described herein and subjected to methods to liberate and collect their protein and/or nucleic acid contents. For example, in several embodiments, exosomes are lysed with a detergent (or non-detergent) based solution in order to disrupt the exosomal membrane and allow for the collection of proteins from the exosome. As discussed above, specific methods can then be optionally employed to identify and selected particularly desired proteins. In several embodiments, nucleic acids are isolated using chaotropic disruption of the exosomes and subsequent isolation of nucleic acids. Other established methods for nucleic acid isolation may also be used in addition to, or in place of chaotropic disruption. Nucleic acids that are isolated may include, but are not limited to DNA, DNA fragments, and DNA plasmids, total RNA, mRNA, tRNA, snRNA, saRNA, miRNA, rRNA, regulating RNA, non-coding and coding RNA, and the like. In several embodiments in which RNA is isolated, the RNA can be used as a template in an RT-PCR-based (or other amplification) method to generate large copy numbers (in DNA form) of the RNA of interest. In such instances, should a particular RNA or fragment be of particular interest, the exosomal isolation and preparation of the RNA can optionally be supplemented by the in vitro synthesis and co-administration of that desired sequence.

In several embodiments, exosomes derived from cells (e.g., exosomes engineered for high potency) are administered in combination with one or more additional agents. For example, in several embodiments, the exosomes are administered in combination with one or more proteins or nucleic acids derived from the exosome (e.g., to supplement the exosomal contents). In several embodiments, the cells from which the exosomes are isolated are administered in conjunction with the exosomes. In several embodiments, such an approach advantageously provides an acute and more prolonged duration of exosome delivery (e.g., acute based on the actual exosome delivery and prolonged based on the cellular delivery, the cells continuing to secrete exosomes post-delivery).

In several embodiments, exosomes (e.g., exosomes engineered for high potency) are delivered in conjunction with a more traditional therapy, e.g., surgical therapy or pharmaceutical therapy. In several embodiments such combinations of approaches result in synergistic improvements in the viability and/or function of the target tissue. In some embodiments, exosomes may be delivered in conjunction with a gene therapy vector (or vectors), nucleic acids (e.g., those used as siRNA or to accomplish RNA interference), and/or combinations of exosomes derived from other cell types.

The compositions disclosed herein can be administered by one of many routes, depending on the embodiment. For example, exosome administration may be by local or systemic administration. Local administration, depending on the tissue to be treated, may in some embodiments be achieved by direct administration to a tissue (e.g., direct injection, such as intramyocardial injection). Local administration may also be achieved by, for example, lavage of a particular tissue (e.g., intra-intestinal or peritoneal lavage). In several embodiments, systemic administration is used and may be achieved by, for example, intravenous and/or intra-arterial delivery. In certain embodiments, intracoronary delivery is used. In several embodiments, the exosomes are specifically targeted to the damaged or diseased tissues. In some such embodiments, the exosomes are modified (e.g., genetically or otherwise) to direct them to a specific target site. For example, modification may, in some embodiments, comprise inducing expression of a specific cell-surface marker on the exosome, which results in specific interaction with a receptor on a desired target tissue. In one embodiment, the native contents of the exosome are removed and replaced with desired exogenous proteins or nucleic acids. In one embodiment, the native contents of exosomes are supplemented with desired exogenous proteins or nucleic acids. In some embodiments, however, targeting of the exosomes is not performed. In several embodiments, exosomes are modified to express specific nucleic acids or proteins, which can be used, among other things, for targeting, purification, tracking, etc. In several embodiments, however, modification of the exosomes is not performed. In some embodiments, the exosomes do not comprise chimeric molecules.

In some embodiments, subcutaneous or transcutaneous delivery methods are used. Due to the relatively small size, exosomes are particularly advantageous for certain types of therapy because they can pass through blood vessels down to the size of the microvasculature, thereby allowing for significant penetration into a tissue. In some embodiments, this allows for delivery of the exosomes directly to central portion of the damaged or diseased tissue (e.g., to the central portion of a tumor or an area of infarcted cardiac tissue). In addition, in several embodiments, use of exosomes is particularly advantageous because the exosomes can deliver their payload (e.g., the resident nucleic acids and/or proteins) across the blood brain barrier, which has historically presented an obstacle to many central nervous system therapies. In certain embodiments, however, exosomes may be delivered to the central nervous system by injection through the blood brain barrier. In several embodiments, exosomes are particularly beneficial for administration because they permit lower profile delivery devices for administration (e.g., smaller size catheters and/or needles). In several embodiments, the smaller size of exosomes enables their navigation through smaller and/or more convoluted portions of the vasculature, which in turn allows exosomes to be delivered to a greater portion of most target tissues.

The dose of exosomes administered, depending on the embodiment, ranges from about 1.0×10⁵ to about 1.0×10⁹ exosomes, including about 1.0×10⁵ to about 1.0×10⁶, about 1.0×10⁶ to about 1.0×10⁷, about 1.0×10⁷ to about 5.0×10⁷, about 5.0×10⁷ to about 1.0×10⁸, about 1.0×10⁸ to about 2.0×10⁸, about 2.0×10⁸ to about 3.5×10⁸, about 3.5×108 to about 5.0×10⁸, about 5.0×10⁸ to about 7.5×10⁸, about 7.5×10⁸ to about 1.0×10⁹, and overlapping ranges thereof. In certain embodiments, the exosome dose is administered on a per kilogram basis, for example, about 1.0×10⁵ exosomes/kg to about 1.0×10⁹ exosomes/kg. In additional embodiments, exosomes are delivered in an amount based on the mass of the target tissue, for example about 1.0×10⁵ exosomes/gram of target tissue to about 1.0×10⁹ exosomes/gram of target tissue. In several embodiments, exosomes are administered based on a ratio of the number of exosomes the number of cells in a particular target tissue, for example exosome: target cell ratio ranging from about 109:1 to about 1:1, including about 10⁸:1, about 10⁷:1, about 10⁶:1, about 10⁵:1, about 10⁴:1, about 10³:1, about 10²:1, about 10:1, and ratios in between these ratios. In additional embodiments, exosomes are administered in an amount about 10-fold to an amount of about 1,000,000-fold greater than the number of cells in the target tissue, including about 50-fold, about 100-fold, about 500-fold, about 1000-fold, about 10,000-fold, about 100,000-fold, about 500,000-fold, about 750,000-fold, and amounts in between these amounts. If the exosomes are to be administered in conjunction with the concurrent therapy (e.g., cells that can still shed exosomes, pharmaceutical therapy, nucleic acid therapy, and the like) the dose of exosomes administered can be adjusted accordingly (e.g., increased or decreased as needed to achieve the desired therapeutic effect). Advantageously, the engineered high-potency exosomes disclosed herein allow for reduced doses of exosomes to be used, in several embodiments with enhanced therapeutic effects despite the lower dose.

In several embodiments, the exosomes are delivered in a single, bolus dose. In some embodiments, however, multiple doses of exosomes may be delivered. In certain embodiments, exosomes can be infused (or otherwise delivered) at a specified rate over time. In several embodiments, when exosomes are administered within a relatively short time frame after an adverse event (e.g., an injury or damaging event, or adverse physiological event such as an MI), their administration prevents the generation or progression of damage to a target tissue. For example, if exosomes are administered within about 20 to about 30 minutes, within about 30 to about 40 minutes, within about 40 to about 50 minutes, within about 50 to about 60 minutes post-adverse event, the damage or adverse impact on a tissue is reduced (as compared to tissues that were not treated at such early time points). In some embodiments, the administration is as soon as possible after an adverse event. In some embodiments the administration is as soon as practicable after an adverse event (e.g., once a subject has been stabilized in other respects). In several embodiments, administration is within about 1 to about 2 hours, within about 2 to about 3 hours, within about 3 to about 4 hours, within about 4 to about 5 hours, within about 5 to about 6 hours, within about 6 to about 8 hours, within about 8 to about 10 hours, within about 10 to about 12 hours, and overlapping ranges thereof. Administration at time points that occur longer after an adverse event are effective at preventing damage to tissue, in certain additional embodiments.

As discussed above, exosomes provide, at least in part, a portion of the indirect tissue regeneration effects seen as a result of certain cellular therapies. Thus, in some embodiments, delivery of exosomes (alone or in combination with an adjunct agent such as nucleic acid) provide certain effects (e.g., paracrine effects) that serve to promote repair of tissue, improvement in function, increased viability, or combinations thereof. In some embodiments, the protein content of delivered exosomes is responsible for at least a portion of the repair or regeneration of a target tissue. For example, proteins that are delivered by exosomes may function to replace damaged, truncated, mutated, or otherwise mis-functioning or nonfunctional proteins in the target tissue. In some embodiments, proteins delivered by exosomes, initiate a signaling cascade that results in tissue repair or regeneration. In several embodiments, miRNA delivery by exosomes is responsible, in whole or in part, for repair and/or regeneration of damaged tissue. As discussed above, miRNA delivery may operate to repress translation of certain messenger RNA (for example, those involved in programmed cell death), or may result in messenger RNA cleavage. In either case, and in some embodiments, in combination, these effects alter the cell signaling pathways in the target tissue and, as demonstrated by the data disclosed herein, can result in improved cell viability, increased cellular replication, beneficial anatomical effects, and/or improved cellular function, each of which in turn contributes to repair, regeneration, and/or functional improvement of a damaged or diseased tissue as a whole.

### Causes of Damage or Disease

The methods and compositions disclosed herein can be used to repair or regenerate cells or tissues affected by a wide variety of types of damage or disease. The compositions and methods disclosed herein can be used to treat inherited diseases, cellular or body dysfunctions, combat normal or abnormal cellular ageing, induce tolerance, modulate immune function. Additionally, cells or tissues may be damaged by trauma, such as blunt impact, laceration, loss of blood flow and the like. Cells or tissues may also be damaged by secondary effects such as post-injury inflammation, infection, auto-digestion (for example, by proteases liberated as a result of an injury or trauma). The methods and compositions disclosed herein can also be used, in certain embodiments, to treat acute events, including but not limited to, myocardial infarction, spinal cord injury, stroke, and traumatic brain injury. In several embodiments, the methods and compositions disclosed herein can be used to treat chronic diseases, including but not limited to neurological impairments or neurodegenerative disorders (e.g., multiple sclerosis, amyotrophic lateral sclerosis, heat stroke, epilepsy, Alzheimer's disease, Parkinson's disease, Huntington's disease, dopaminergic impairment, dementia resulting from other causes such as AIDS, cerebral ischemia including focal cerebral ischemia, physical trauma such as crush or compression injury in the CNS, including a crush or compression injury of the brain, spinal cord, nerves or retina, and any other acute injury or insult producing neurodegeneration), immune deficiencies, facilitation of repopulation of bone marrow (e.g., after bone marrow ablation or transplantation), arthritis, auto-immune disorders, inflammatory bowel disease, cancer, diabetes, muscle weakness (e.g., muscular dystrophy, amyotrophic lateral sclerosis, and the like), progressive blindness (e.g. macular degeneration), and progressive hearing loss.

In several embodiments, the damaged tissue comprises one or more of neural and/or nervous tissue, epithelial tissue, skeletal muscle tissue, endocrine tissue, vascular tissue, smooth muscle tissue, liver tissue, pancreatic tissue, lung tissue, intestinal tissue, osseous tissue, connective tissue, or combinations thereof. In several embodiments, the damaged tissue is in need of repair, regeneration, or improved function due to an acute event. Acute events include, but are not limited to, trauma such as laceration, crush or impact injury, shock, loss of blood or oxygen flow, infection, chemical or heat exposure, poison or venom exposure, drug overuse or overexposure, and the like. For example, in several embodiments, the damaged tissue is cardiac tissue and the acute event comprises a myocardial infarction. In some embodiments, administration of the exosomes results in an increase in cardiac wall thickness in the area subjected to the infarction. In additional embodiments, the tissue is damaged due to chronic disease or ongoing injury. For example, progressive degenerative diseases can lead to tissue damage that propagates over time (at times, even in view of attempted therapy). Chronic disease need not be degenerative to continue to generate damaged tissue, however. In several embodiments, chronic disease/injury includes, but it not limited to epilepsy, Alzheimer's disease, Parkinson's disease, Huntington's disease, dopaminergic impairment, dementia, ischemia including focal cerebral ischemia, ensuing effects from physical trauma (e.g., crush or compression injury in the CNS), neurodegeneration, immune hyperactivity or deficiency, bone marrow replacement or functional supplementation, arthritis, auto-immune disorders, inflammatory bowel disease, cancer, diabetes, muscle weakness (e.g., muscular dystrophy, amyotrophic lateral sclerosis, and the like), blindness and hearing loss. Cardiac tissue, in several embodiments, is also subject to damage due to chronic disease, such as for example congestive heart failure, ischemic heart disease, diabetes, valvular heart disease, dilated cardiomyopathy, infection, and the like. Other sources of damage also include, but are not limited to, injury, age-related degeneration, cancer, and infection. In several embodiments, the regenerative cells are from the same tissue type as is in need of repair or regeneration. In several other embodiments, the regenerative cells are from a tissue type other than the tissue in need of repair or regeneration. In several embodiments, the regenerative cells comprise somatic cells, while in additional embodiments, they comprise germ cells. In still additional embodiments, combinations of one or more cell types are used to obtain exosomes (or the contents of the exosomes).

In several embodiments, exosomes can be administered to treat a variety of cancerous target tissues, including but not limited to those affected with one or of acute lymphoblastic leukemia (ALL), acute myeloid leukemia (AML), adrenocortical carcinoma, kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma, breast cancer, bronchial tumors, burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma hairy cell leukemia, renal cell cancer, leukemia, oral cancer, liver cancer, lung cancer, lymphoma, melanoma, ocular cancer, ovarian cancer, pancreatic cancer, prostate cancer, pituitary cancer, uterine cancer, and vaginal cancer.

Alternatively, in several embodiments, exosomes are delivered to an infected target tissue, such as a target tissue infected with one or more bacteria, viruses, fungi, and/or parasites. In some embodiments, exosomes are used to treat tissues with infections of bacterial origin (e.g., infectious bacteria is selected the group of genera consisting of Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, and Yersinia, and mutants or combinations thereof). In several embodiments, the exosomes inhibit or prevent one or more bacterial functions, thereby reducing the severity and/or duration of an infection. In several embodiments, administration of exosomes sensitizes the bacteria (or other pathogen) to an adjunct therapy (e.g., an antibiotic).

In some embodiments, the infection is viral in origin and the result of one or more viruses selected from the group consisting of adenovirus, Coxsackievirus, Epstein-Barr virus, hepatitis a virus, hepatitis b virus, hepatitis c virus, herpes simplex virus type 1, herpes simplex virus type 2, cytomegalovirus, ebola virus, human herpes virus type 8, HIV, influenza virus, measles virus, mumps virus, human papillomavirus, parainfluenza virus, poliovirus, rabies virus, respiratory syncytial virus, rubella virus, and varicella-zoster virus. Exosomes can be used to treat a wide variety of cell types as well, including but not limited to vascular cells, epithelial cells, interstitial cells, musculature (skeletal, smooth, and/or cardiac), skeletal cells (e.g., bone, cartilage, and connective tissue), nervous cells (e.g., neurons, glial cells, astrocytes, Schwann cells), liver cells, kidney cells, gut cells, lung cells, skin cells or any other cell in the body.

### Therapeutic Compositions

In several embodiments, there are provided compositions comprising cells for use in repair or regeneration of tissues that have been adversely impacted by damage or disease. In several embodiments, there are provided compositions comprising exosomes (e.g., exosomes engineered for high potency) for use in repair or regeneration of tissues that have been adversely impacted by damage or disease. In several embodiments, the compositions comprise, consist of, or consist essentially of exosomes. In some embodiments, the exosomes comprise nucleic acids, proteins, or combinations thereof. In several embodiments, the nucleic acids within the exosomes comprise one or more types of RNA (though certain embodiments involved exosomes comprising DNA). The RNA, in several embodiments, comprises one or more of messenger RNA, snRNA, saRNA, miRNA, and combinations thereof. In several embodiments, the miRNA comprises one or more of miR-92a, miR-26a, miR27-a, let-7e, mir-19b, miR-125b, mir-27b, let-7a, miR-19a, let-7c, miR-140-3p, miR-125a-5p, miR-150, miR-155, mir-210, let-7b, miR-24, miR-423-5p, miR-22, let-7f, miR-146a, and combinations thereof. In several embodiments, the compositions comprise, consist of, or consist essentially of a synthetic microRNA and a pharmaceutically acceptable carrier. In some such embodiments, the synthetic microRNA comprises miR146a. In several embodiments the miRNA is pre-miRNA (e.g., not mature), while in some embodiments, the miRNA is mature, and in still additional embodiments, combinations of pre-miRNA and mature miRNA are used.

In several embodiments, the compositions comprise exosomes (e.g., exosomes engineered for high potency) derived from a population of cells, as well as one or more cells from the population (e.g., a combination of exosomes and their “parent cells”). In several embodiments, the compositions comprise a plurality of exosomes derived from a variety of cell types (e.g., a population of exosomes derived from a first and a second type of “parent cell”). As discussed above, in several embodiments, the compositions disclosed herein may be used alone, or in conjunction with one or more adjunct therapeutic modalities (e.g., pharmaceutical, cell therapy, gene therapy, protein therapy, surgery, etc.).

In several embodiments, the exosomes are about 15 nm to about 95 nm in diameter, including about 15 nm to about 20 nm, about 20 nm to about 25 nm, about 25 nm to about 30 nm, about 30 nm to about 35 nm, about 35 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm and overlapping ranges thereof In certain embodiments, larger exosomes are obtained are larger in diameter (e.g., those ranging from about 140 to about 210 nm). Advantageously, in several embodiments, the exosomes comprise synthetic membrane bound particles (e.g., exosome surrogates), which depending on the embodiment, are configured to a specific range of diameters. In such embodiments, the diameter of the exosome surrogates is tailored for a particular application (e.g., target site or route of delivery). In still additional embodiments, the exosome surrogates are labeled or modified to enhance trafficking to a particular site or region post-administration.

In several embodiments, exosomes are obtained via centrifugation of the regenerative cells. In several embodiments, ultracentrifugation is used. However, in several embodiments, ultracentrifugation is not used. In several embodiments, exosomes are obtained via size-exclusion filtration of the regenerative cells. As disclosed above, in some embodiments, synthetic exosomes are generated, which can be isolated by similar mechanisms as those above.

In several embodiments, the exosomes induce altered gene expression by repressing translation and/or cleaving mRNA, for example. In some embodiments, the alteration of gene expression results in inhibition of undesired proteins or other molecules, such as those that are involved in cell death pathways, or induce further damage to surrounding cells (e.g., free radicals). In several embodiments, the alteration of gene expression results directly or indirectly in the creation of desired proteins or molecules (e.g., those that have a beneficial effect). The proteins or molecules themselves need not be desirable per se (e.g., the protein or molecule may have an overall beneficial effect in the context of the damage to the tissue, but in other contexts would not yield beneficial effects). In some embodiments, the alteration in gene expression causes repression of an undesired protein, molecule or pathway (e.g., inhibition of a deleterious pathway). In several embodiments, the alteration of gene expression reduces the expression of one or more inflammatory agents and/or the sensitivity to such agents. Advantageously, the administration of exosomes, or miRNAs, in several embodiments, results in downregulation of certain inflammatory molecules and/or molecules involved in inflammatory pathways. As such, in several embodiments, cells that are contacted with the exosomes or miRNAs enjoy enhanced viability, even in the event of post-injury inflammation or inflammation due to disease.

In several embodiments, the exosomes fuse with one or more recipient cells of the damaged tissue. In several embodiments, the exosomes release the microRNA into one or more recipient cells of the damaged tissue, thereby altering at least one pathway in the one or more cells of the damaged tissue. In some embodiments, the exosomes exerts their influence on cells of the damaged tissue by altering the environment surrounding the cells of the damaged tissue. In some embodiments, signals generated by or as a result of the content or characteristics of the exosomes, lead to increases or decreases in certain cellular pathways. For example, the exosomes (or their contents/characteristics) can alter the cellular milieu by changing the protein and/or lipid profile, which can, in turn, lead to alterations in cellular behavior in this environment. Additionally, in several embodiments, the miRNA of an exosome can alter gene expression in a recipient cell, which alters the pathway in which that gene was involved, which can then further alter the cellular environment. In several embodiments, the influence of the exosomes directly or indirectly stimulates angiogenesis. In several embodiments, the influence of the exosomes directly or indirectly affects cellular replication. In several embodiments, the influence of the exosomes directly or indirectly inhibits cellular apoptosis.

The beneficial effects of the exosomes (or their contents) need not only be on directly damaged or injured cells. In some embodiments, for example, the cells of the damaged tissue that are influenced by the disclosed methods are healthy cells. However, in several embodiments, the cells of the damaged tissue that are influenced by the disclosed methods are damaged cells.

In several embodiments, regeneration comprises improving the function of the tissue. For example, in certain embodiments in which cardiac tissue is damaged, functional improvement may comprise increased cardiac output, contractility, ventricular function and/or reduction in arrhythmia (among other functional improvements). For other tissues, improved function may be realized as well, such as enhanced cognition in response to treatment of neural damage, improved blood-oxygen transfer in response to treatment of lung damage, improved immune function in response to treatment of damaged immunological-related tissues.

In several embodiments, the regenerative cells and/or exosomes are mammalian in origin. In several embodiments, the regenerative cells and/or exosomes are human in origin. In some embodiments, the cells and/or exosomes are non-embryonic human regenerative cells and/or exosomes. In several embodiments, the regenerative cells and/or exosomes are autologous to the individual while in several other embodiments the regenerative cells and/or exosomes are allogeneic to the individual. Xenogeneic or syngeneic cells and/or exosomes are used in certain other embodiments.

### Materials and Methods for Examples 1-10

### Cells and Reagents

Endomyocardial biopsies from the right ventricular aspect of the interventricular septum were obtained from the healthy hearts of deceased tissue donors. CDCs were derived as described previously. Briefly, heart biopsies were minced into small 1 mm² fragments and digested briefly with collagenase. Explants were then cultured on 20 g/ml fibronectin (VWR)-coated flasks. Stromal-like, flat cells, and phase-bright round cells grew spontaneously from the tissue fragments and reached confluence by two to three weeks. These cells were then harvested using 0.25% trypsin (GIBCO) and cultured in suspension on 20 μg/ml poly-D-lysine (BD Biosciences) to form self-aggregating cardiospheres. CDCs were obtained by seeding cardiospheres onto fibronectin-coated dishes and passaged. All cultures were maintained at 5% 02/CO₂ at 37 °C, using IMDM basic media (GIBCO) supplemented with 10% FBS (Hyclone), 1% Gentamicin, and 0.1 ml 2-mercaptoethanol. Human heart biopsy specimens, from which CDCs were grown, were obtained under a protocol approved by the institutional review board for human subjects research.

### Extracellular Vesicle Preparation and Isolation

Extracellular Vesicles were harvested from primary CDCs at passage 5 or older passages from transduced cells using a hypoxic cycling method used previously. Briefly, cells were grown to confluence at 20% O₂/5% CO₂ at 37 °C, and then cells were serum-free at 2% O₂/5% CO₂ at 37 °C overnight after one wash. Conditioned media was collected and filtered through 0.45 μm filter to remove apoptotic bodies and cellular debris and frozen for later use at −80 °C. EVs were purified using centrifugal ultrafiltration with a 1000 KDa molecular weight cutoff filter (Sartorius). EV preparations were analyzed through Malvern Nanosight NS300 Instrument (Malvern Instruments) with the following acquisition parameters: camera levels of 15, detection level less than or equal to 5, number of videos taken 4, and video length of 30 s.

### Lentiviral Transduction

CDCs or NHDFs were plated in T25 flasks and transduced with lentiviral particles (MOI: 20) in complete media. After 24 hrs transduction, virus was removed, and fresh complete media was added for cell recovery for a further 24 hrs.

Cells were then subjected to selection media for approximately one week. Following selection, complete media was replaced.

RNA Isolation and qRT-PCR

Cell RNA was isolated using a miRNeasy Mini Kit (Qiagen). Exosome RNA was isolated using the Urine Exosome RNA Isolation Kit (Norgen Biotek Corp.). Reverse transcription was performed using High Capacity RNA to cDNA (Thermo Fisher Scientific) or TaqMan® microRNA Reverse Transcription Kit (Applied Biosystems) for RNA and micro RNA, respectively. Real-time PCR was performed using TaqMan Fast Advanced Master Mix and the appropriate TaqMan® Gene Expression Assay (Thermo Fisher Scientific). Samples were processed and analyzed using a QuantStudio™ 12K Flex Real-Time PCR system and each reaction was performed in triplicate samples (with housekeeping genes hprt1 for mRNA and miR23a for microRNA). The gene expression assays/microRNAs used in this study were as follows (Thermo Fisher Scientific):

| Assay Names | Species | Assay Numbers |
|---|---|---|
| ctnnb1 | Human | Hs00355049_m1 |
| ext1 | Human | Hs00609162_m1 |
| extl1 | Human | Hs00184929_m1 |
| gata4 | Human | Hs00171403_m1 |
| gsk3b | Human | Hs01047719_m1 |
| hprt1 | Human | Hs02800695_m1 |
| lrp5 | Human | Hs01124561_m1 |
| lrp6 | Human | Hs00233945_m1 |
| mest | Human | Hs00853380_g1 |
| nkx2.5 | Human | Hs00231763_m1 |
| tert | Human | Hs00972656_m1 |
| miR22-5p | Human | 000398 |
| miR23a-3p | Human | 000399 |
| miR26a-3p | Human | 000405 |
| miR146a-5p | Human | 000468 |
| miR199b-5p | Human | 000500 |
| hsa-miR-335-3p | Human | 000546 |

### RNA Sequencing

Cell and exosome RNA samples were sequenced at the Cedars Sinai Genomics Core. Total RNA and Small RNA were analyzed using an Illumina NextSeq 500 platform for cell and exosome samples respectively.

### Cell Lysate and Protein Assay

Cell lysates were collected for ELISA and western blot. For ELISA, 4×10⁵ cells were collected and pelleted at 1,000 rpm for 5 min at 4 °C. Cell pellets were lysed in 1× lysis buffer (Affymetrix eBioscience InstantOne ELISA kit) and incubated for 10 min at room temperature with regular agitation. For western blot, cells were pelleted and resuspended in 1× RIPA buffer (Alfa Aesar) with protease inhibitor on ice for 30 min. Protein lysates were isolated by centrifugation at 14,000 rpm for 15 min at 4 °C. Protein concentration was measured using a DC™ Protein Assay kit (Bio-Rad).

### Drug Exposure of Cells

Cells were exposed to 5 μM of 6-bromoindirubin-3′-oxime (BIO, Sigma-Aldrich) or 4-Benzyl-2-(naphthalene-1-yl)-[1,2,4]thiadiazolidine-3,5-dione (Tideglusib, Sigma-Aldrich) for 48 or 72 hours in complete media.

### ELISA

Total β-catenin ELISA was performed according the protocol described with a final sample concentration of 0.01 mg/ml and positive control of 0.1 mg/ml (Affymetrix eBioscience InstantOne™ ELISA).

### Flow Cytometry

Cells were harvested and counted (2×10⁵ cells per condition). Cells were washed with 1% bovine serum albumin (BSA) in 1× phosphate-buffered saline (PBS) and stained with the appropriate antibody (BD Pharmingen) for 1 hr at 4 °C. Cells were then washed again and resuspended in 1% BSA in 1×PBS. BD Cytofix/Cytoperm™ kit was used for cell permeabilization before staining. Flow analysis was done using a BD FACS Canto™ II instrument.

### Western Blot

Membrane transfer was performed using the Turbo® Transfer System (BIO-RAD) after gel electrophoresis. The following antibody staining was then applied and detected by SuperSignal™ West Pico PLUS Chemiluminescent Substrate (Thermo Fisher Scientific).

| | Primary/ | | Catalog |
|---|---|---|---|
| Antibody Names | Secondary | Company | Numbers |
| Pan-Actin | Primary | Cell Signaling | 12748 |
| (D18C11) Rabbit | | Technology | |
| mAb-HRP | | | |
| Conjugated | | | |
| GAPDH Rabbit | Primary | Cell Signaling | 14C10 |
| mAb-HRP | | Technology | |
| Conjugated | | | |
| Anti-Mest Rabbit | Primary | Abcam | ab230114 |
| Polyclonal Antibody | | | |
| EXTL1 Polyclonal | Primary | Thermo Fisher | PA5-72069 |
| Antibody | | Scientific | |
| Anti-Rabbit IgG, | Secondary | Cell Signaling | 7074 |
| HRP-Linked | | Technology | |
| Antibody | | | |

### Animal Study

All animal studies were conducted under approved protocols from the Institutional Animal Care and Use Committee protocols.

### Mouse Acute MI Model

Acute myocardial infarction was induced in three-month-old male severe combined immunodeficient (SCID)/beige mice (n=5-7 animals per group). Within 10 min of coronary ligation, 1×10⁵ cells, EVs, drugs (or vehicle) were injected intramyocardially.

Echocardiography. Echocardiography study was performed in the SCID/beige at 24 hr (baseline) and three weeks after surgery using Vevo 3100 or 770 Imaging System (Visual Sonics) as described. The average of the left ventricular ejection fraction was analyzed from multiple left ventricular end-diastolic and left ventricular end-systolic measurements.

CDC Engraftment. To assess human CDC persistence, infarcted animals received LP CDCs pre-exposed to 5 μM of BIO or an equivalent volume of DMSO 72 hours prior to injection. A standard curve was made using copy numbers of the human X-chromosome specific gene mage al. DNA from known numbers of this CDC donor in DNA from 1 mg of mouse cardiac tissue was used to make the standard curve. Three weeks post-injection animals were sacrificed, and genomic DNA was extracted from ventricular tissue. QPCR of mage al copy number in genomic DNA was done using a Taqman Copy Number Assay (Thermo Fisher Scientific).

### Histology

Animals were sacrificed 3 weeks after MI induction. Hearts were harvested and a transverse cut was made slightly above the MI suture. The apical portion was then embedded in optimum cutting temperature solution in a base mold/embedding ring block (Tissue Tek). Blocks were immediately frozen by submersion in cold 2-methylbutane. Hearts were sectioned at a thickness of 5 μm.

### Masson's Trichrome Staining

Two slides containing a total of four sections per heart were stained using Masson's trichrome stain. In brief, sections were treated overnight in Bouin's solution. Slides were then rinsed for 10 min under running water and stained with Weigert's hematoxylin for 5 min. Slides were then rinsed and stained with scarlet-acid fuchsin for 5 min and rinsed again. Slides were then stained with phosphotungstic/phosphomolybdic, aniline blue, and 2% acetic acid for 5 min each. Slides were then rinsed, dried, and mounted using DPX mounting media.

### Duchenne Muscular Dystrophy Mouse Model

### Treadmill Exercise Testing

Ten-month-old female mdx mice were placed inside an Exer-3/6 rodent treadmill (Columbus Instruments) equipped with a shock grid elevated 5 degrees. During the acclimatization period, mice were undisturbed for 30 min prior to engagement of the belt. After the belt engaged, mice were encouraged to familiarize themselves with walking on the treadmill at a pace of 10 m/min for an additional 20 min. After the acclimatization period, the exercise protocol engaged (shock grid activated at 0.15 mA with a frequency of 1 shock/sec). This protocol is intended to induce volitional exhaustion by accelerating the belt speed by 1 m/min every minute. Mice that rest on the shock grid for >10 s with nudging were considered to have reached their maximal exercise capacity (their accumulated distance traveled is recorded) and the exercise test was terminated. Animals were tested at baseline, then later in the day received 100 μl intravenous (femoral vein) infusions of exosomes or saline vehicle. Animals were tested one more time three weeks post infusion.

### Histology

The mouse tibialis anterior (TA) muscles were dissected freely from anesthetized mice and embedded in OCT compound and frozen in 2-methylbutane pre-cooled in liquid nitrogen, then stored at −80 °C until sectioning. Serial sections were cut at the mid-belly in the transverse plane. All sections were cut at 8 μm using a cryostat (Leica) and adhered to Superfrost Plus™ microscope slides (Fisherbrand). Cryosections were fixed with 10% neutral buffered formalin for 10 min prior to Masson's trichrome staining (Sigma-Aldrich). Histological slides were imaged using an Aperio AT Turbo slide scanner (Leica) at 40× magnification. Quantification of fibrosis was determined by the area of blue staining relative to red staining of the entire tissue section using Tissue IA (Leica Biosystems). Feret diameter was measured on 1,000 myofibers per section using QuPath software integrated with ImageJ.

### Statistical Analysis

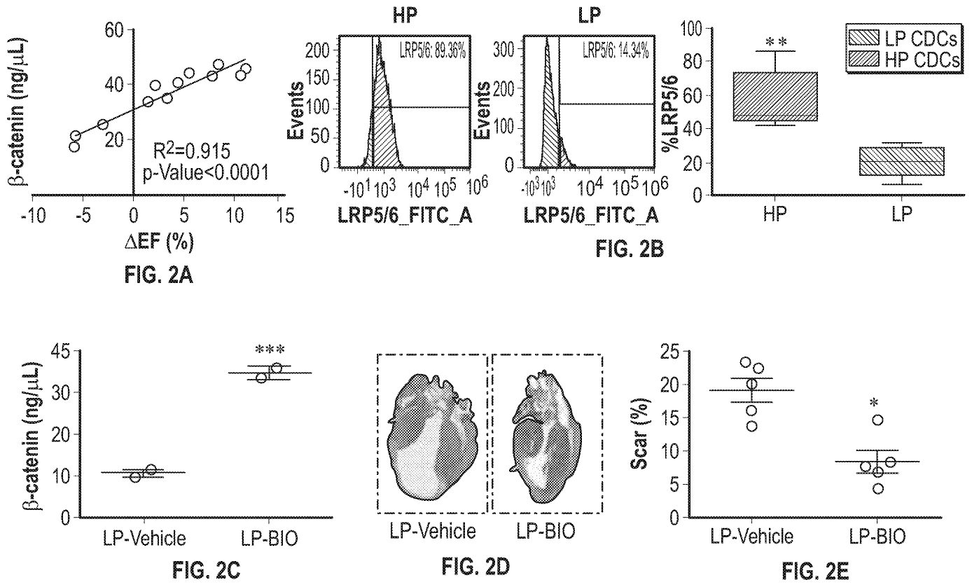

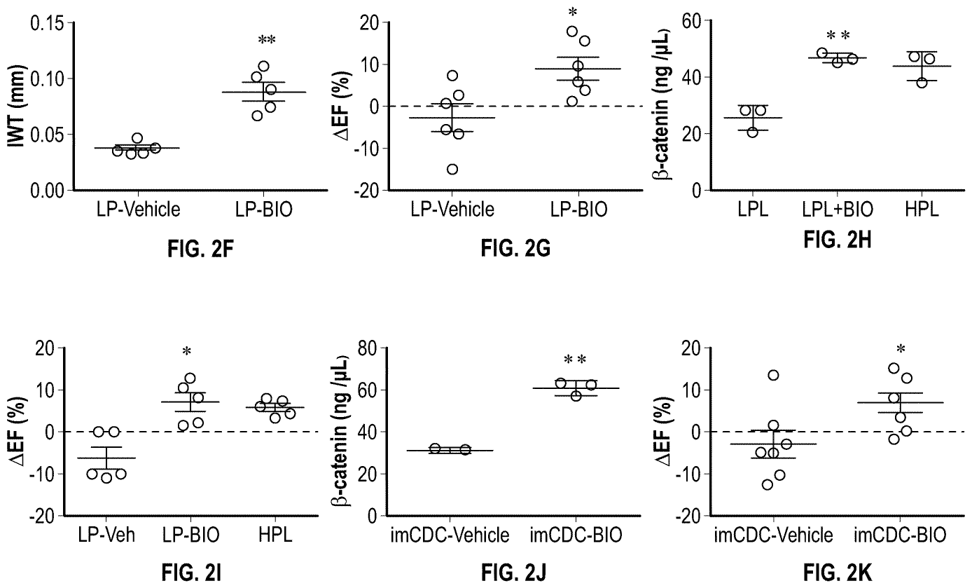

Statistical Comparisons were made using an independent one-tailed or two-tailed independent Student's T-test with a 95% CI. A univariate regression analysis was used in FIG. 2A.

### Example 1

This non-limiting example describes the implication of Wnt/β-catenin signaling in CDC therapeutic potency.

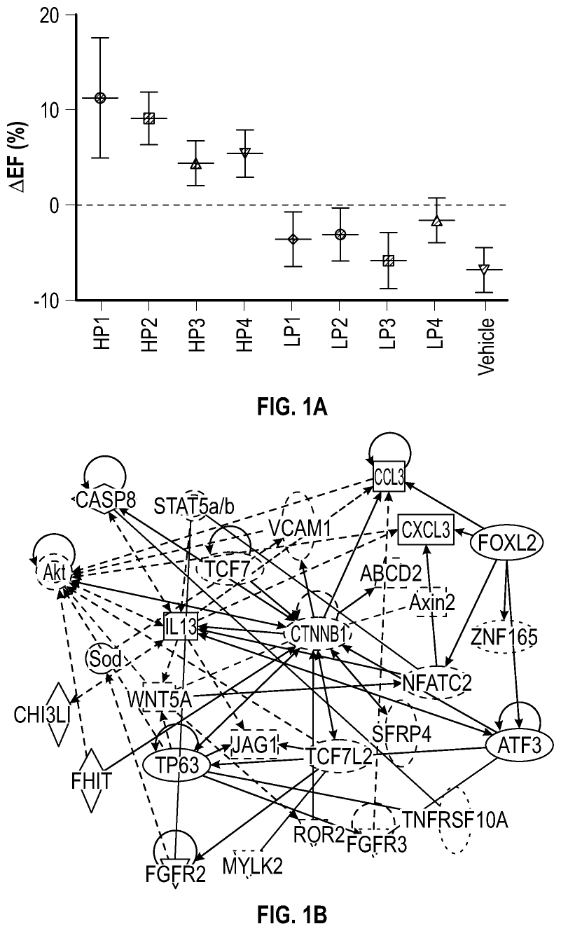

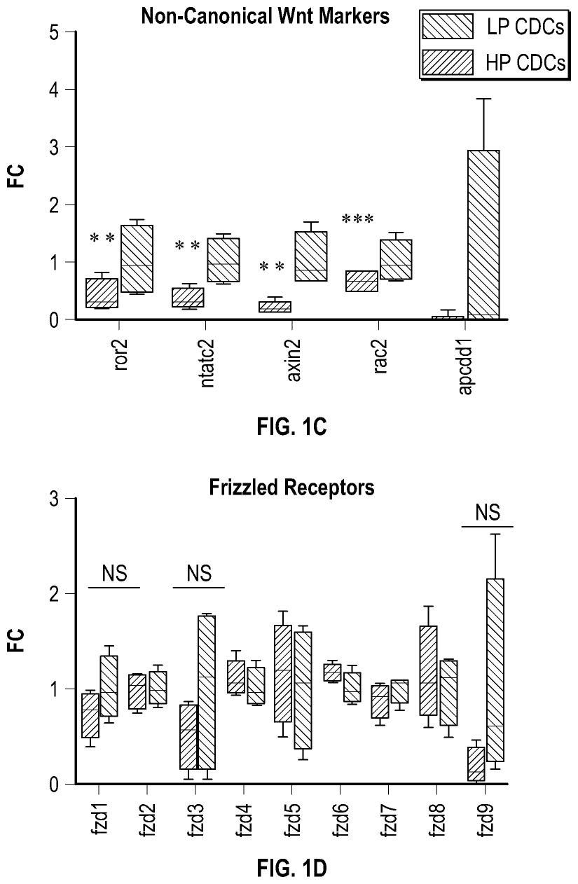

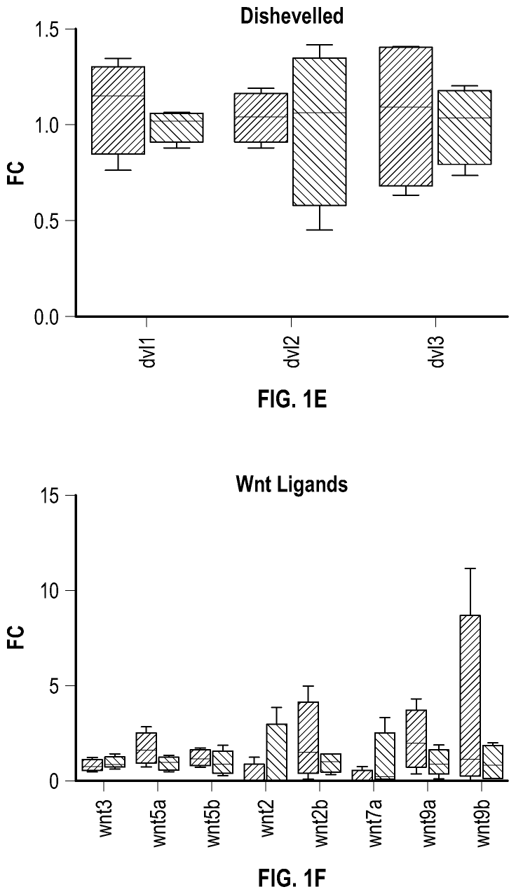

Variable therapeutic efficacy is evident among various human CDC lines subjected to in vivo testing post-MI. FIG. 1A shows the changes in global heart function, quantified echocardiographically as ejection fraction (EF), from mice injected with each of four high-potency (HP) human CDC lines, four low-potency (LP) lines (selected for sequencing), or vehicle only (saline). Transcriptomic comparison of HP and LP CDCs revealed differentially-expressed Wnt signaling mediators, with activation of β-catenin signaling in HP CDCs (FIG. 1B). In contrast, non-canonical Wnt pathway members ror2, nfatc2, axin2, rac2, and apcdd1 were enriched in LP CDCs (FIG. 1C), while little difference was evident in several molecules that are shared by canonical and non-canonical Wnt signaling pathways (Frizzled receptors (FIG. 1D), Dishevelled, (FIG. 1E) and Wnt ligands (FIG. 1F)).

Based on RNA sequencing results, the relationship between Wnt/β-catenin signaling and CDC potency were examined. Pooled data for donor-specific total β-catenin protein levels in CDCs revealed a strong correlation with therapeutic efficacy of the same cells in vivo (FIG. 2A). All CDCs were from putatively healthy donor hearts which had passed standard minimal criteria for human transplantation (including screening for infectious diseases) but had not been used for a technical reason (e.g., heart size, blood type) and thus were donated for research. No discernible correlation was found between clinical characteristics of donors (i.e. age, sex, ethnicity, or cause of death) and the observed potency of CDCs. HP CDCs exhibited ~2-fold higher β-catenin levels, on average, compared with LP CDCs. Wnt receptor expression, including low-density lipoprotein receptor 5/6 (LRP5/6), promotes stabilization of cytoplasmic β-catenin and prevents its ubiquitination. Wnt receptors LRP5/6 were elevated in HP CDCs (FIG. 2B).

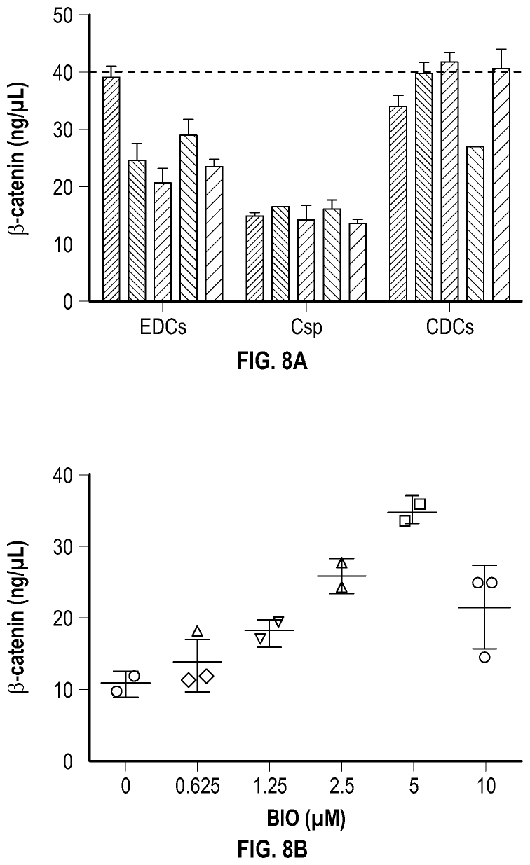

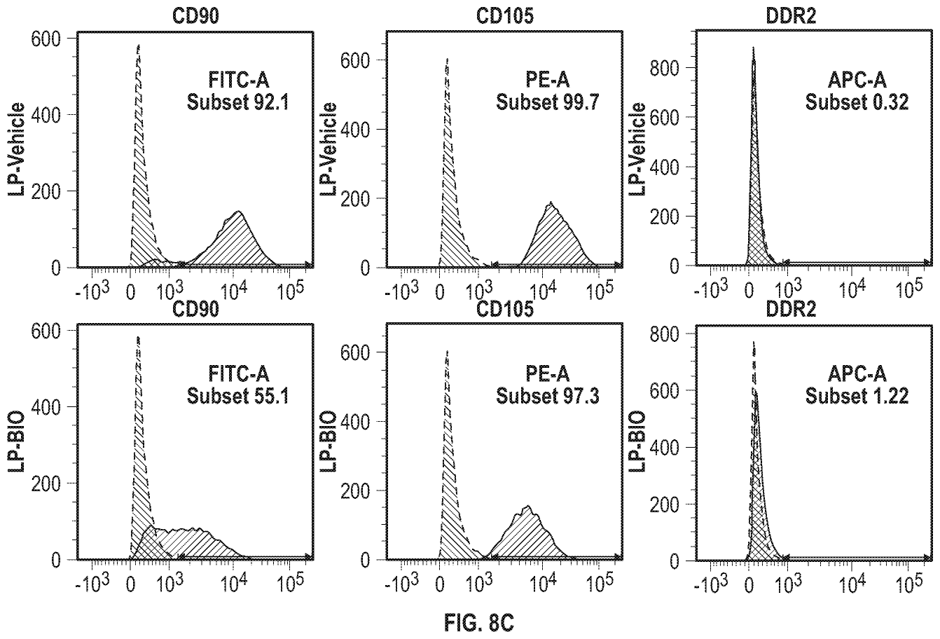

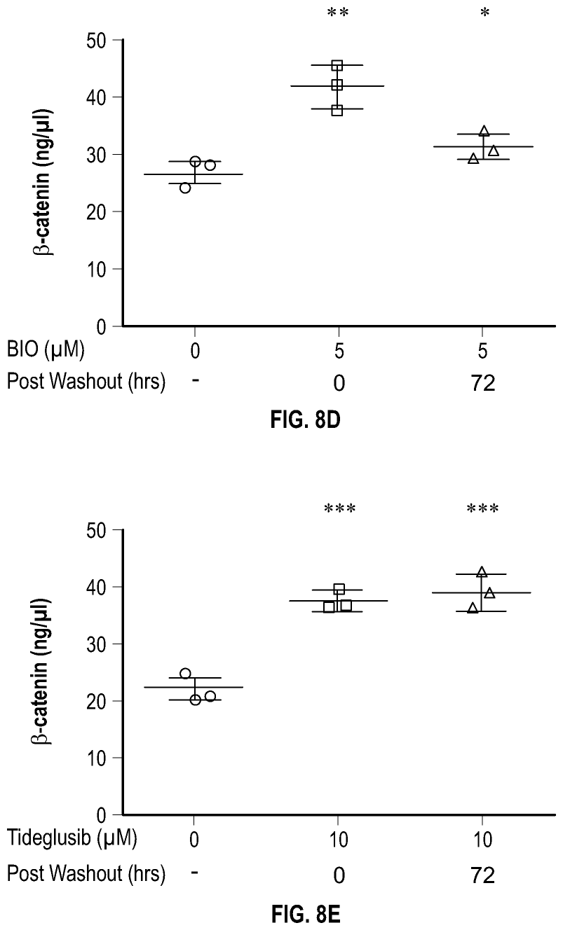

Furthermore, the sphere-forming transition, central to the preparation of CDCs, involves a dramatic decrease then sharp rise of β-catenin levels in the CDCs thereafter (though variability among donors was observed) (FIG. 8A).

These results show the role of Wnt/β-catenin signaling in CDC therapeutic potency. In some embodiments, β-catenin levels are upregulated or increased in HP CDCs. In some embodiments, upregulation of β-catenin levels enhances therapeutic potency of CDCs. In some embodiments, Wnt receptors LRP5/6 are upregulated in HP CDCs. In some embodiments, upregulation of Wnt receptors LRP5/6 enhances therapeutic potency of CDCs.

### Example 2

This non-limiting example shows that boosting β-catenin enhances therapeutic potency.

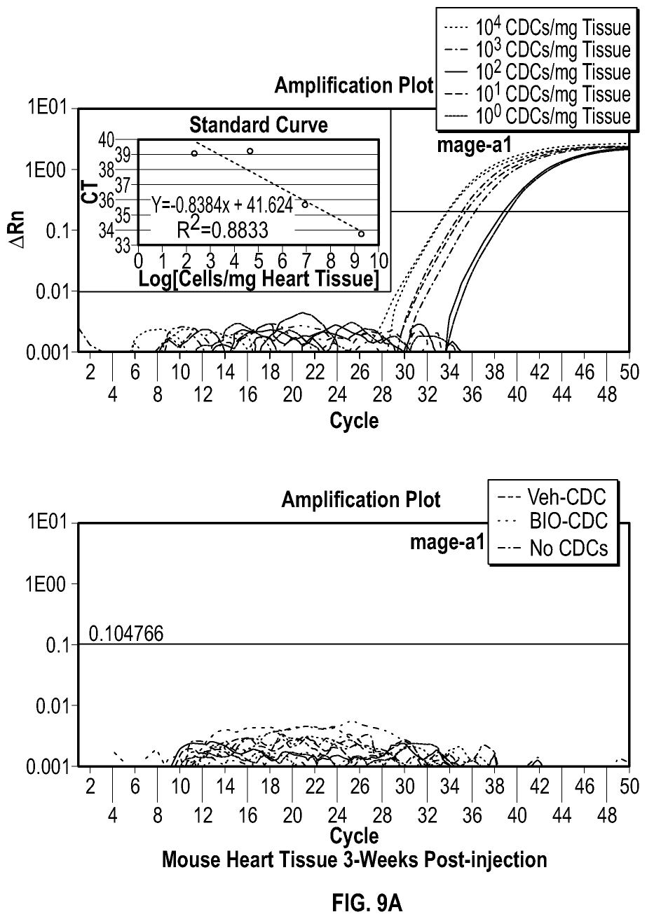

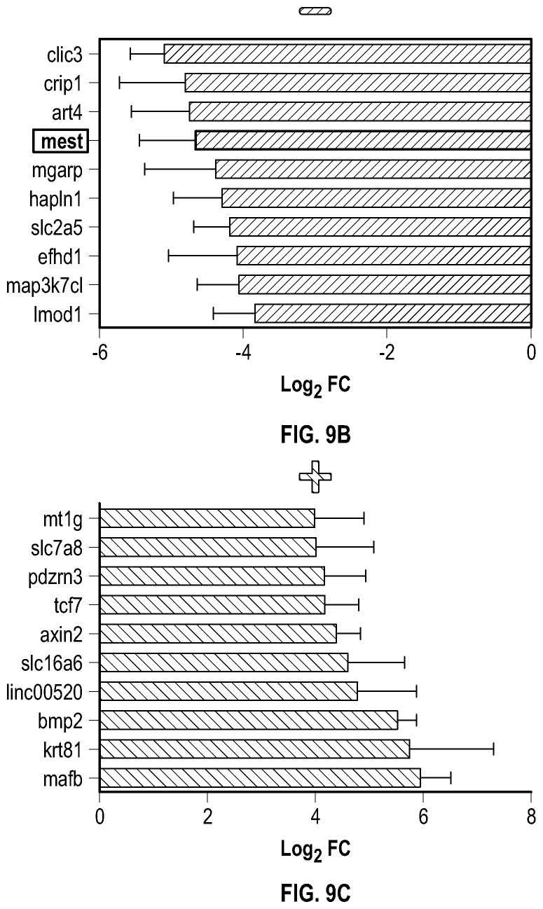

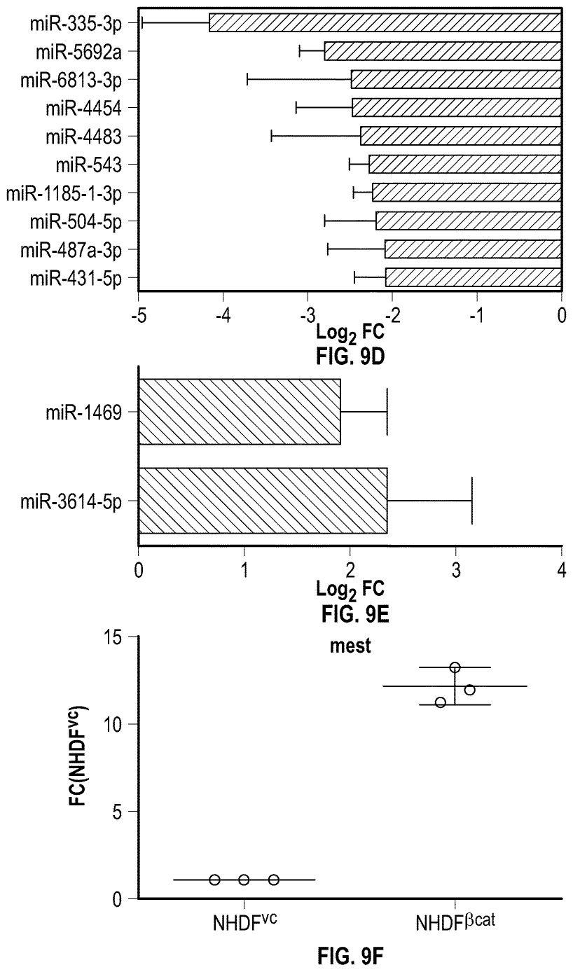

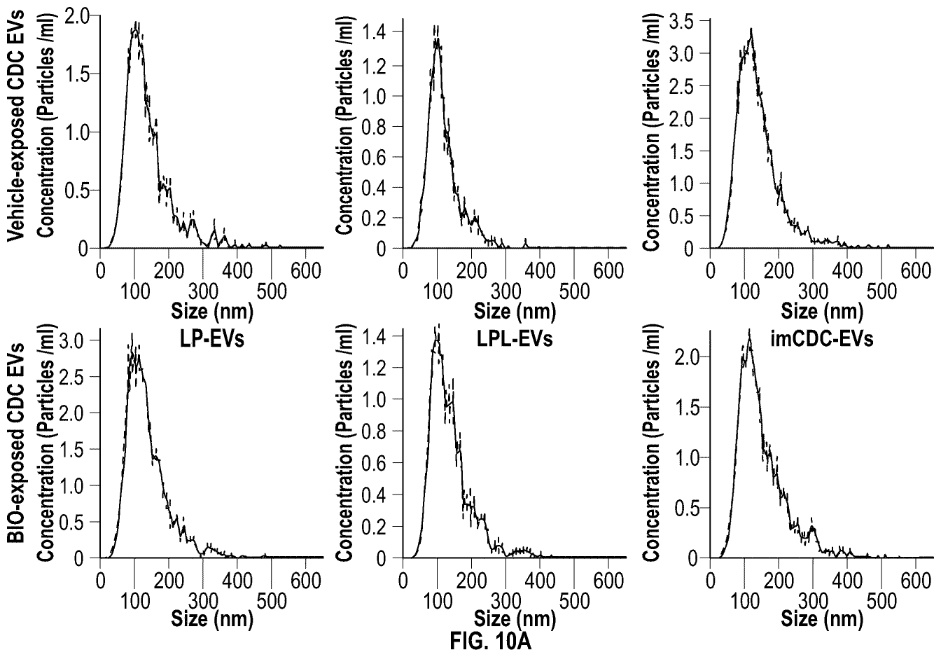

To test whether boosting β-catenin levels would improve therapeutic efficacy in LP CDCs, 6-bromoindirubin-3′-oxime (BIO), a reversible inhibitor of glycogen synthase kinase-3 beta (GSK3β) which is maximally effective in CDCs at 5 μM, was used (FIG. 8B). By releasing GSK3β's suppressive effect, BIO can increase β-catenin levels, which was indeed observed in a LP line exposed to BIO (LP-BIO, FIG. 2C). BIO decreased the expression of CD90, an antigen which correlates inversely with potency, without affecting the positive CDC identity marker CD105 or the negative identity marker DDR2 (FIG. 8C). Tideglusib, an irreversible inhibitor of GSK3β, had directionally similar but longer-lasting effects (FIGS. 8D and 8E). LP-BIO CDCs showed enhanced functional and structural benefits compared to unexposed LP CDCs (LP-Vehicle) (FIGS. 2D-2G). Enhancement of β-catenin did not affect the persistence of transplanted CDCs in host cardiac tissue (FIG. 9A).

In some instances, donor-to-donor variability in potency occurs and occasionally, different lots from the same master cell bank can differ in potency. According to several embodiments, variability in potency between lots from the same master cell bank is limited. FIG. 2H shows that β-catenin levels increase when LP lots (LPL) are exposed to BIO (LPL-BIO), and do so to levels comparable to HP lots (HPL) from the same donor. Such “corrected” lots also regain therapeutic efficacy in vivo (FIG. 2I). Finally, CDCs immortalized using simian virus 40 large and small T antigen (SV40 T+t) were not potent and exhibit low levels of β-catenin, but regain potency following β-catenin augmentation by exposure to BIO (FIGS. 2J, 2K). Thus, in three different scenarios-donor-to-donor variability, lot-to-lot variability, and immortalization-boosting CDC β-catenin levels increases cell potency.

In some embodiments, inhibition of GSK3β enhances potency of CDCs. In some embodiments, inhibition of GSK3β enhances β-catenin levels. In some embodiments, inhibition of GSK3β enhances β-catenin levels, thereby enhancing potency of CDCs.

### Example 3

This non-limiting example describes inhibition of mest expression and increased LRP5/6 receptor surface expression upon activation of Wnt/β-catenin signaling.

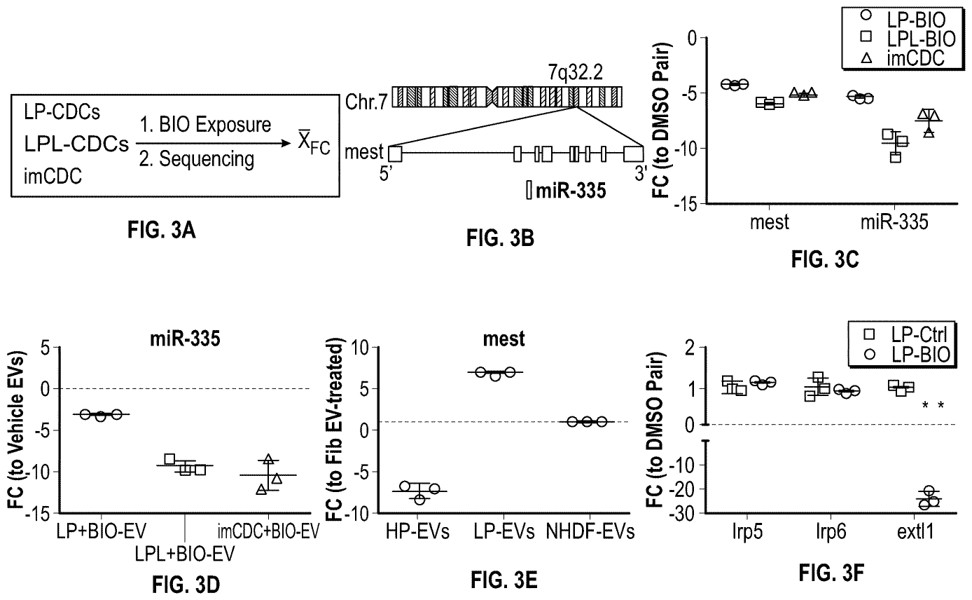

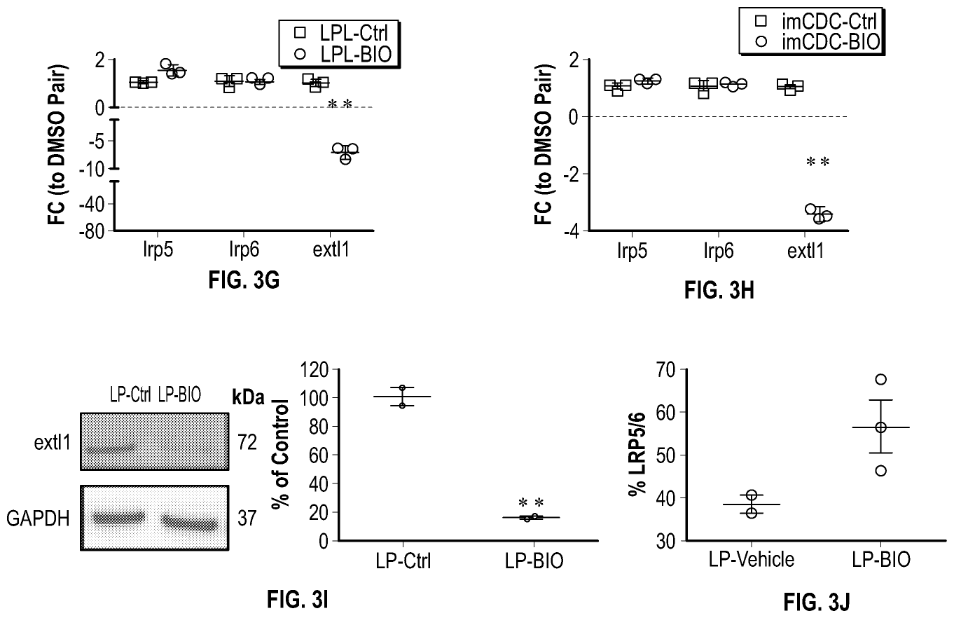

To understand how β-catenin drives potency, the transcriptomes of LP CDCs to those of the same cell batches after exposure to BIO were compared. As stated above, three scenarios associated with low potency were identified: donor-related, in which all lots from a given donor lack potency; lot-dependent, in which some lots are potent and others are not; and immortalized CDCs (imCDCs). Using RNA sequencing, LP cells from each scenario were compared after exposure to BIO versus vehicle alone. Fold changes were then pooled to identify genes up- or down-regulated by BIO (FIG. 3A). In addition to the many promoters of canonical Wnt signaling which were up-regulated, one basal negative regulator of Wnt signaling, mesoderm-specific transcript (mest), was strikingly downregulated (~30-fold; FIGS. 3B, 3C; FIGS. 9B and 9C). Differential expression of microRNAs (miRs) between the two groups further identified a cognate miR coregulated with mest (miR-335; FIG. 3C, FIGS. 9D and 9E). Overexpressing β-catenin in fibroblasts increased mest expression, suggesting that β-catenin-mediated mest inhibition is cell autonomous (FIG. 9F). Mest modulates Wnt/β-catenin signaling indirectly through glucosyltransferases that prevent LRP5/6 receptor maturation. Mutations in members of the exostosin (EXT) family of glucosyltransferases affect Wnt receptor pattern expression during development. Here, LRP5/6 transcripts were unchanged with downregulation of the exostosin glycosyltransferase EXTL1, confirming that mest and EXTL1 inhibit LRP5/6 post-transcriptionally (FIGS. 3F-3H). In further support of a mechanistic link, CDC exposure to BIO decreased EXTL1 protein levels (FIG. 3I) and upregulated its glycosylation target LRP5/6 (although that difference was not statistically significant; FIG. 3J).

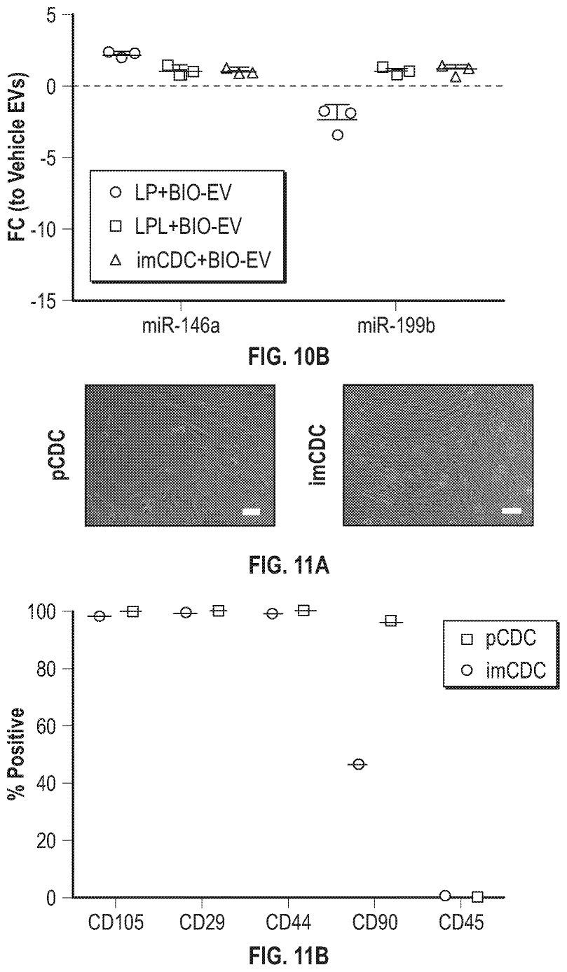

Given the importance of exosomes, and possibly other EVs, as mediators of the therapeutic benefits of CDCs, EV properties and effects were investigated. Despite similar levels of previously-identified positive and negative therapeutic miRs (146a and 199b respectively), and similar size distribution profiles, of EVs produced by plus/minus BIO cell pairs (FIGS. 10A and 10B), EV levels of miR-335 decreased significantly, demonstrating modulation of noncoding RNA payload by β-catenin activation (FIG. 3D). Fibroblasts exposed to HP CDC EVs exhibited downregulated mest levels compared to those exposed to fibroblast EVs or LP CDC EVs (FIG. 3E). Therefore, β-catenin activation leads to mest/miR-335 repression in potent CDCs and decreases miR-335 in their secreted EVs.

These results show that mest inhibition of β-catenin occurs through modulation of LRP5/6 receptor expression. In some embodiments, LRP5/6 receptor expression and/or function can be modulated to further enhance the potency-inducing effects of β-catenin. For example, in some embodiments, expression of the LRP5/6 receptor is upregulated. In some embodiments, mest is downregulated. In some embodiments LRP5/6 receptor expression and/or function is upregulated as a result of mest downregulation.

### Example 4

This non-limiting example describes restoring therapeutic potency by genetic suppression of mest in immortalized CDCs.

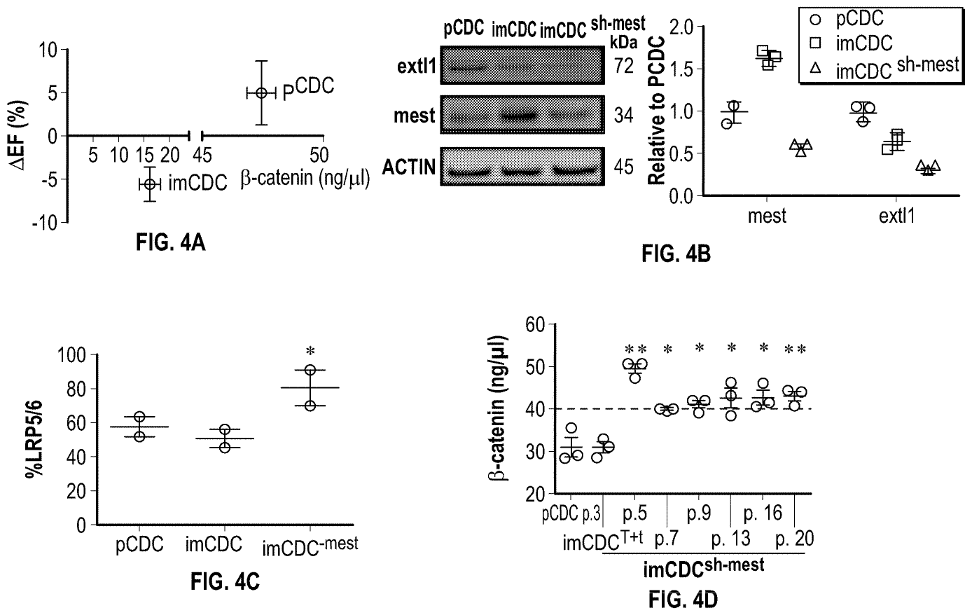

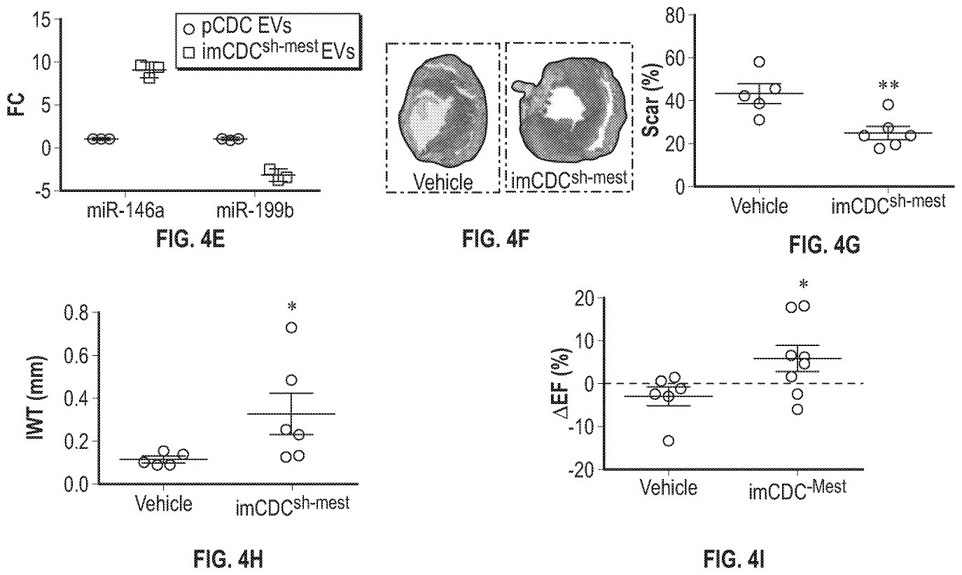

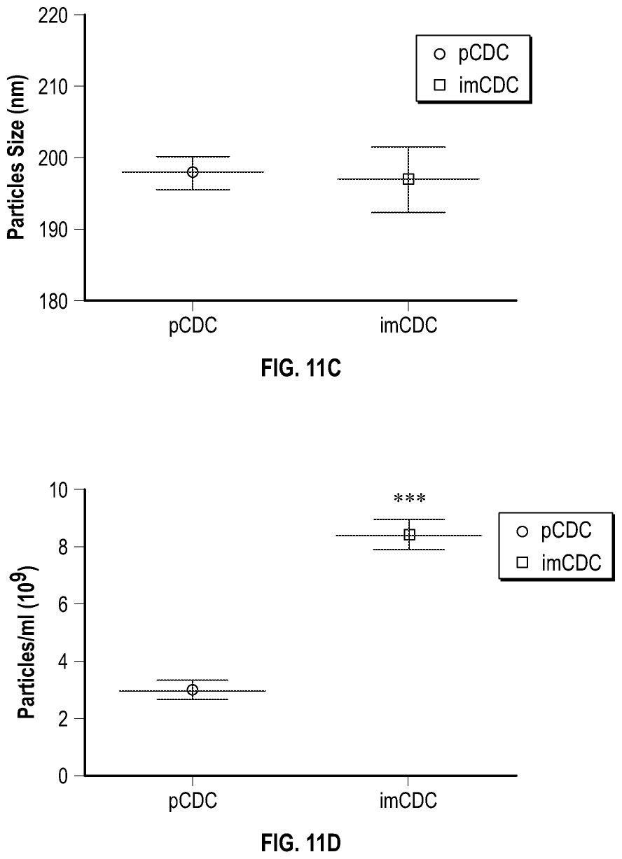

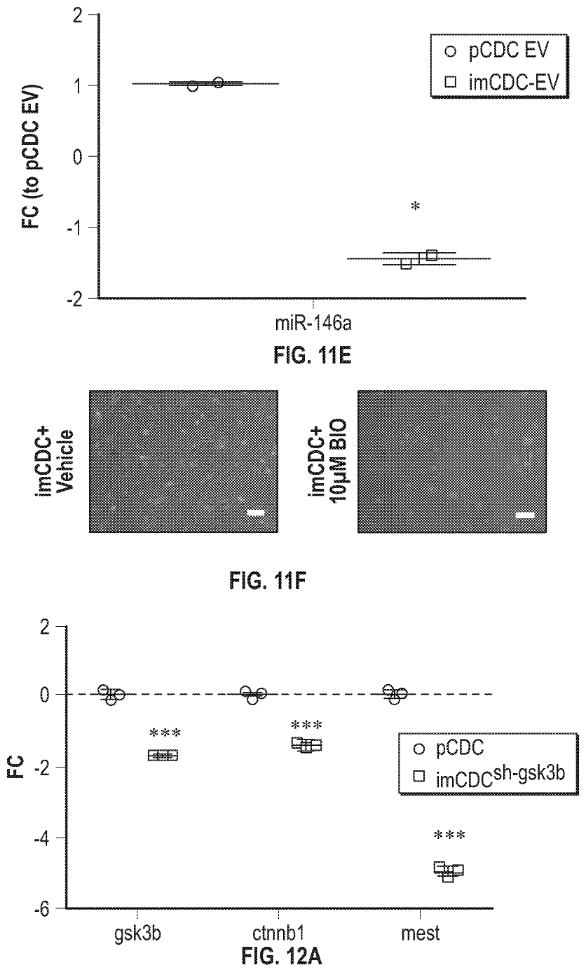

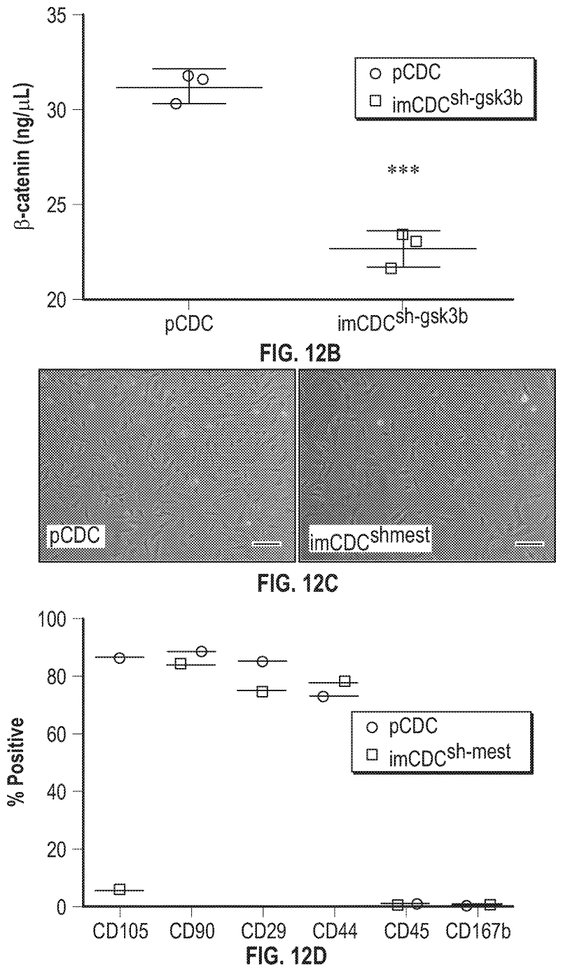

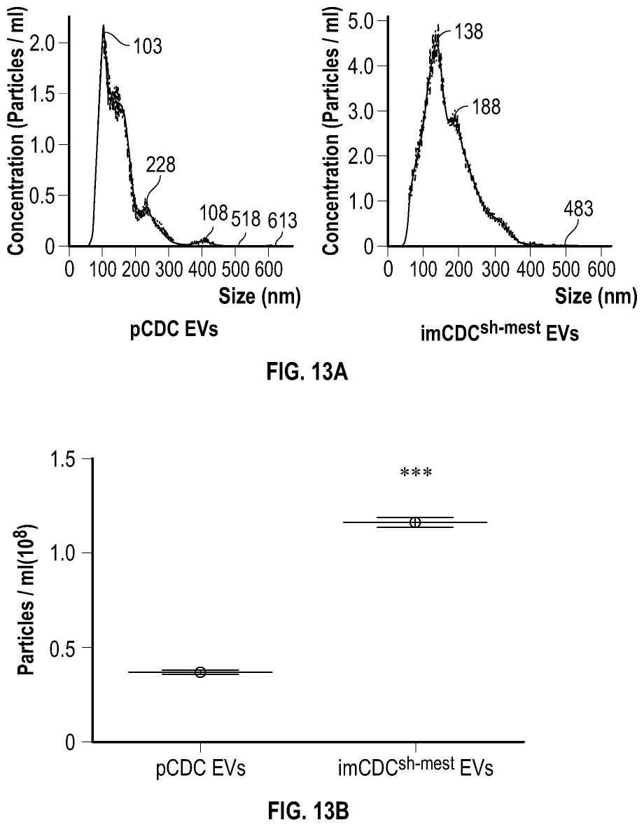

Initial attempts at immortalizing CDCs relied on simian virus 40 large and small T antigen transduction. As expected, using SV40 large and small (T+t) antigen led to a change in morphology towards a spindle-like morphology, and robust growth past the expected ~8 passages post sphere formation (FIG. 11A). Surface marker expression remained largely similar except for a sharp rise in CD90, a previously-identified negative marker of potency in CDCs (FIG. 11B). EV size was similar (FIG. 11C) but EV output was increased; this can be a common consequence of primary cell immortalization (FIG. 11D). Finally, levels of known therapeutic CDC EV cargo components, notably miR-146a and miR-210, fell in comparison to primary CDC EVs (FIG. 11E). Therefore, while this strategy succeeded in immortalizing CDCs, it led to a loss of potency (FIG. 2J, 2K) and attenuation of β-catenin levels (FIG. 4A). Although BIO restored potency in immortalized CDCs (FIG. 2J, 2K), cell growth and viability were undermined (FIG. 11F). In another attempt to restore potency to immortalized CDCs, knockdown of GSK3β led to transcriptional repression (FIG. 12A) and paradoxical downregulation of β-catenin (FIG. 12B). As observed with pharmacological inhibition of GSK3β, transcriptional repression of GSK3β also led to mest downregulation (FIG. 12B). Repression of β-catenin expression was consistent with known homeostatic mechanisms. Gsk3a and gsk3b have functional redundancies, such that blocking gsk3b leads to inhibition of gsk3b-mediated effects; genetic deletion of gsk3b abrogated those effects due to compensatory activation of gsk3a. Genetic suppression of mest using a short hairpin (sh) RNA yielded better results: EXTL1 protein levels decreased, and surface expression of LRP5/6 increased (FIG. 4B, 4C), such that imCDC^{sh-mest} cells maintained high β-catenin levels (comparable to those of potent CDCs) for at least 20 passages (FIG. 4D). While potent therapeutically, imCDC^{sh-mest} differed from primary CDCs in morphology and identity markers (FIGS. 12C, and 12D). EVs were produced by imCDC^{sh-mest} (FIGS. 13A and 13B), and those EVs contained higher miR-146a and lower miR-199b levels than primary CDC EVs (FIG. 4E). Finally, imCDC^{sh-mest} exhibited high potency both structurally (by reductions in histological scar size; FIGS. 4F-4H) and functionally in vivo (FIG. 4I).

These results illustrate that suppression of mest results in high potency CDC and EV. In some embodiments, suppression of mest correlates with decreased EXTL1 protein levels. In some embodiments, suppression of mest correlates with increased surface expression of LRP5/6. In some embodiments, suppression of mest correlates with decreased EXTL1 protein levels and increased surface expression of LRP5/6. In some embodiments, decreased EXTL1 protein levels, increased surface expression of LRP5/6, or both further enhance potency of CDC.

### Example 5

This non-limiting example illustrates engineering therapeutic potency into a non-potent, non-cardiac cell type.

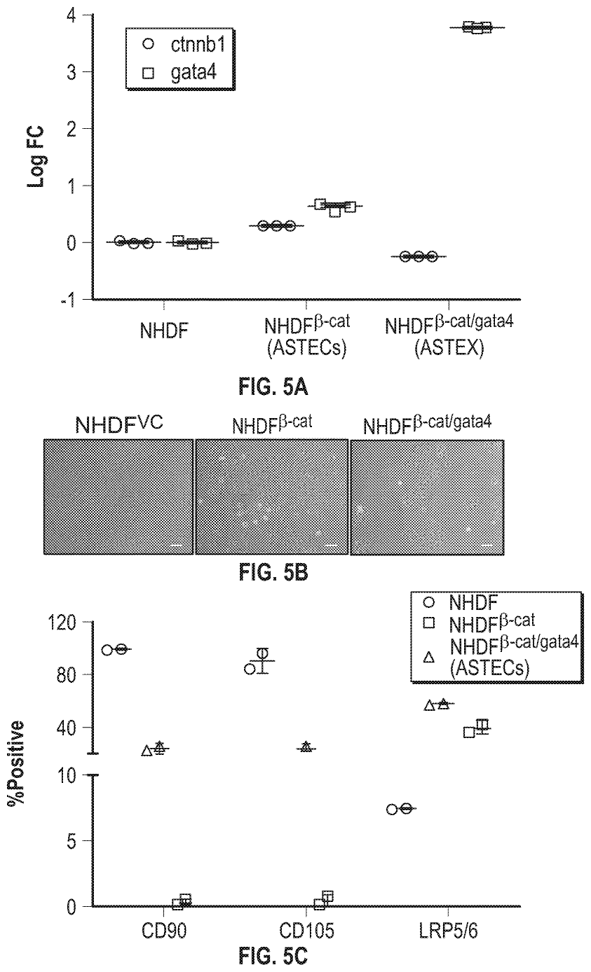

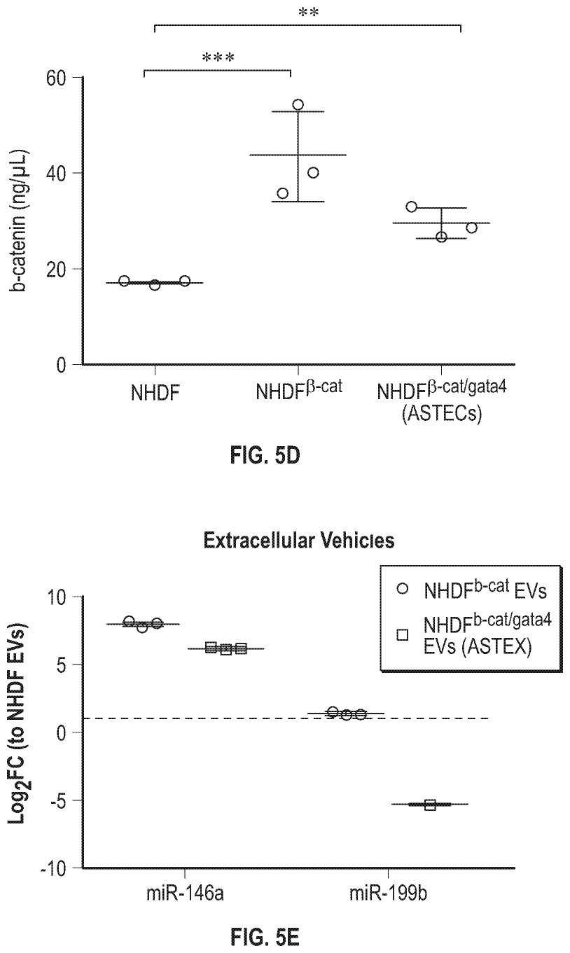

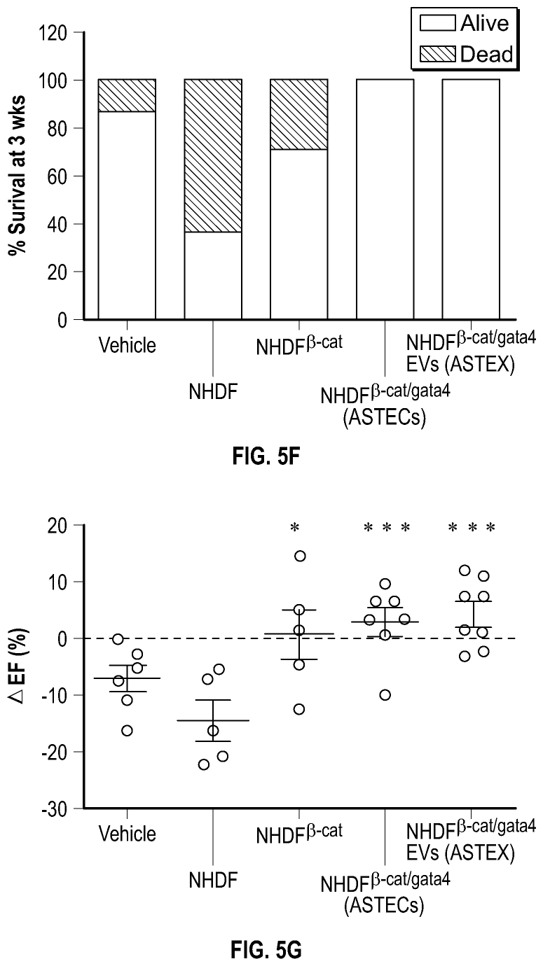

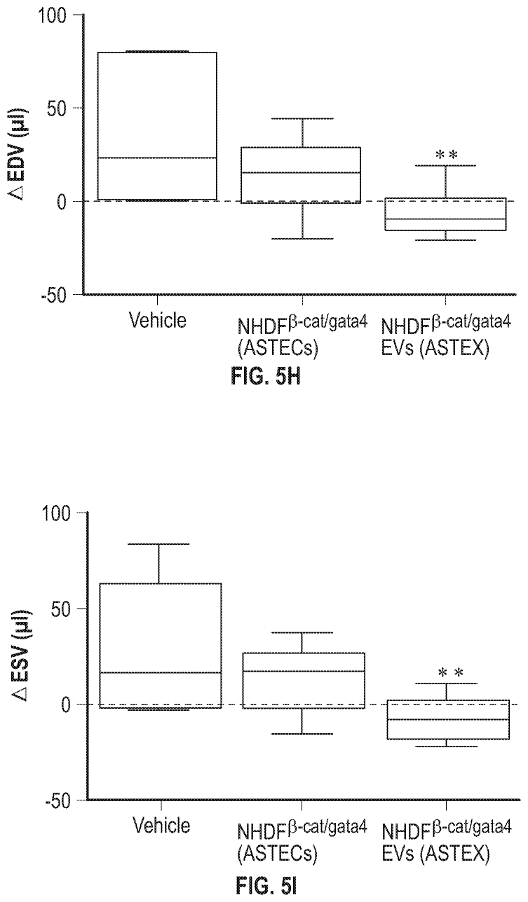

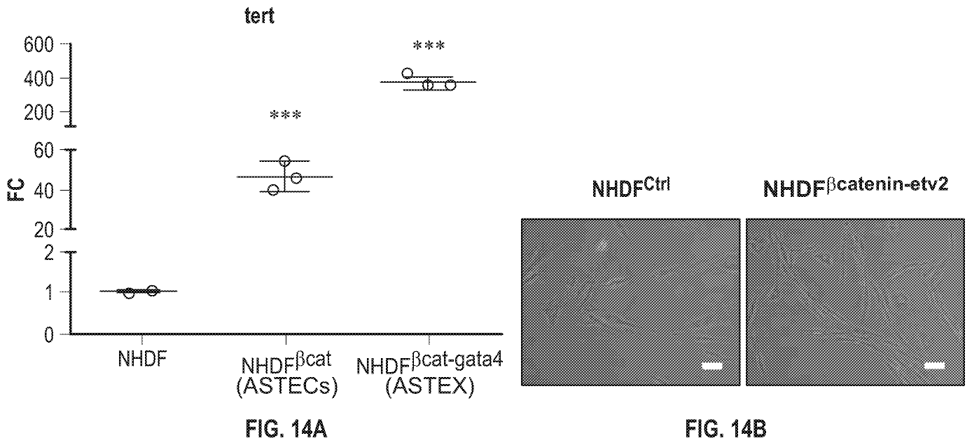

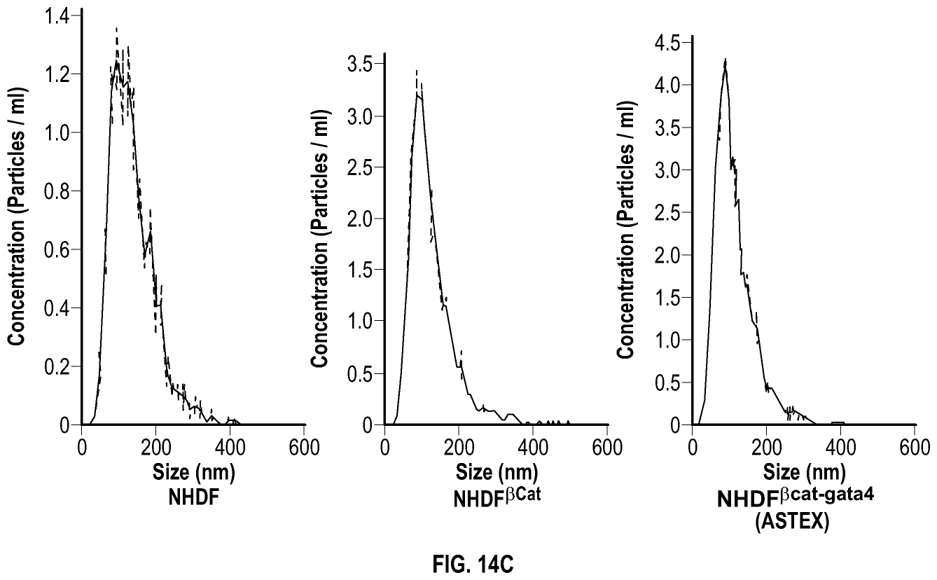

Having shown that β-catenin underlies CDC potency, whether β-catenin overexpression could induce potency in a therapeutically-ineffective cell type, normal human dermal fibroblasts (NHDFs) was investigated. β-catenin enhancement with and without co-expression of gata4 (FIG. 5A), a transcription factor which signals downstream of Wnt/β-catenin during cardiac development and enhances the cardioprotective potential of mesenchymal stem cells, was studied. Comparison of NHDFs, NHDFs transduced with β-catenin only (NHDFβcat), and NHDFs transduced with both β-catenin and gata4 (NHIDF^{βcat/gata4}) revealed clear morphological differences, with NHDFβcat and NHDFβcat/gata4 cells having endothelial- and epithelial-like morphologies, respectively (FIG. 5B). In NHDF^{βcat/gata4}, a lack of senescence akin to immortalization was further observed. Indeed, telomerase expression was markedly increased in these cells, pointing to a possible synergy between β-catenin and gata4 in cell growth (FIG. 14A). Among transcription factors, gata4 is at least somewhat specific in its effects: substituting gata4 with the endothelial cell-fate transcription factor, etv2, did not recapitulate the immortalized phenotype (FIG. 14B). Relative to unmodified NHDFs, antigenic profiling revealed decreases in CD90 and CD105 in NHDFβcat, and almost complete loss of these markers in NHDF^{βcat/gata}4 (FIG. 5C). β-catenin levels were increased in both NHDF^{βcat} and NHDF^{βcat/gata}4 relative to unmodified NHDFs (FIG. 5D), likely due to silencing of β-catenin during cell-fate specification. EVs derived from NHDFβcat and NHDFβcat/gata4 expressed increased levels of miR-146a; however, only NHDF^{βcat/gata}4 showed reduced miR-199b (FIG. 14C; FIG. 5E). To assess therapeutic efficacy, mortality and heart function post-MI was quantified. FIG. 5F shows that NHDFs can be deleterious, not just inert, after transplantation; they hinder survival, insofar as >50% of NHDF-injected animals died by the third week post-MI. Lower mortality was observed in mice injected with NHDFβcat or NHIDF^{βcat/gata4}; indeed, all animals survived in the latter group, and also in a group injected with EVs from NHDF^{βcat/gata}4 (FIG. 5F). Similar patterns characterized the cells' capacity to improve EF post-MI (FIGS. 5G-5I). Given these findings, the engineered cells and their EVs/exosomes were dubbed Activated-Specialized Tissue Effector Cells (ASTECs) or ASTEX, respectively.

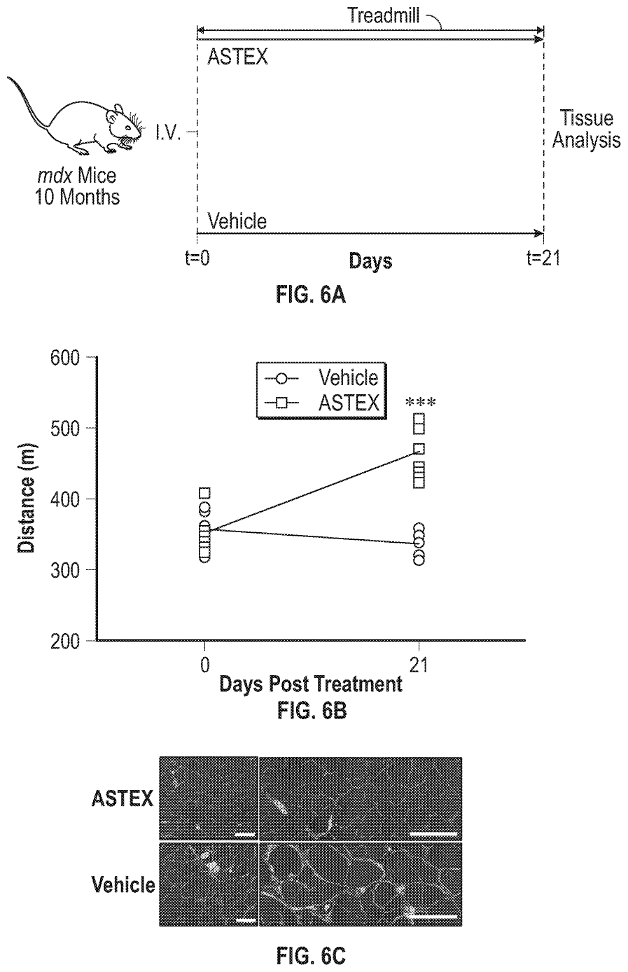

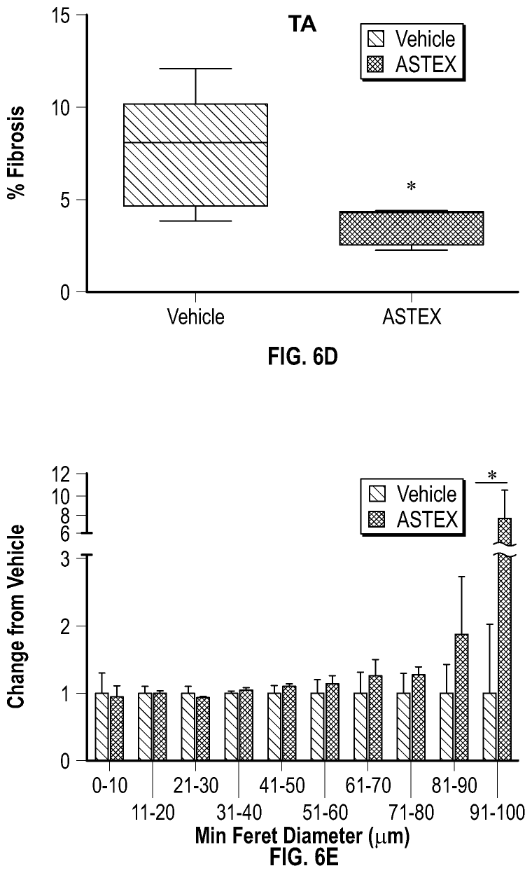

Engineered fibroblasts (or their EVs), ASTECs (or ASTEX), may have therapeutic utility beyond the heart. To probe the bioactivity more generally, ASTEX were tested in a murine model of Duchenne muscular dystrophy by injecting mdx mice with 3×10⁹ particles (or vehicle only) intravenously (FIG. 6A). Three weeks later, ASTEX-injected mice (but not controls) ran significantly further than at baseline (FIG. 6B). Histological examination of the mdx mouse tibialis anterior, a prototypical fast-twitch skeletal muscle, revealed greatly reduced muscle fibrosis in ASTEX relative to control (FIGS. 6C, 6D). Meanwhile, ASTEX shifted myofiber size distribution to larger diameters (FIG. 6E), mimicking the effects of CDC-derived exosomes in this model. Together, these data indicate that ASTEX are bioactive not only in ischemic heart failure (FIG. 5G) but also on dystrophic skeletal muscle.

These results show that therapeutic potency can be engineered into non-potent, non-cardiac cell types by overexpression of β-catenin. In some embodiments, engineered fibroblasts (or their EVs), ASTECs (or ASTEX) are generated by β-catenin enhancement without co-expression of gata4. In some embodiments, engineered fibroblasts (or their EVs), ASTECs (or ASTEX) are generated by β-catenin enhancement with co-expression of gata4.

### Example 6

This non-limiting example shows that the miR-92a-bmp2 signaling axis underlies therapeutic effects of β-catenin activation.

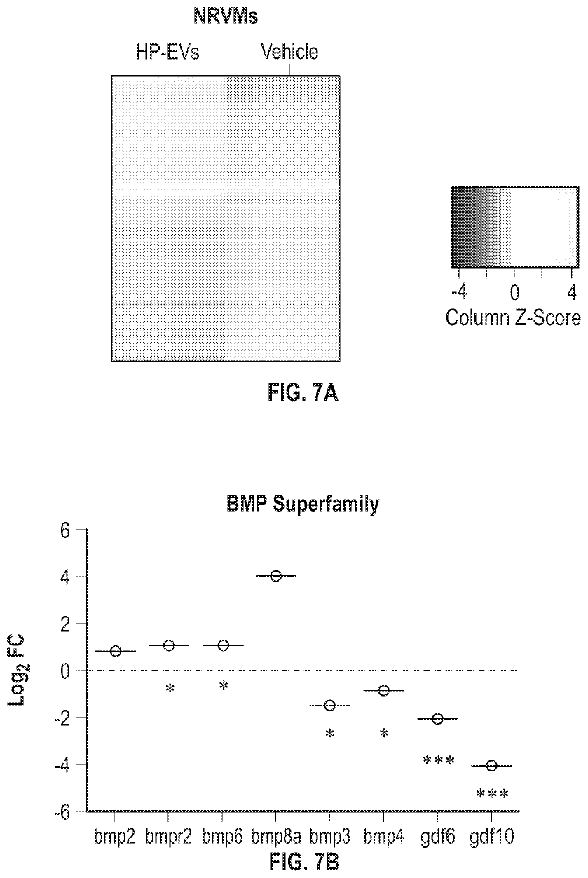

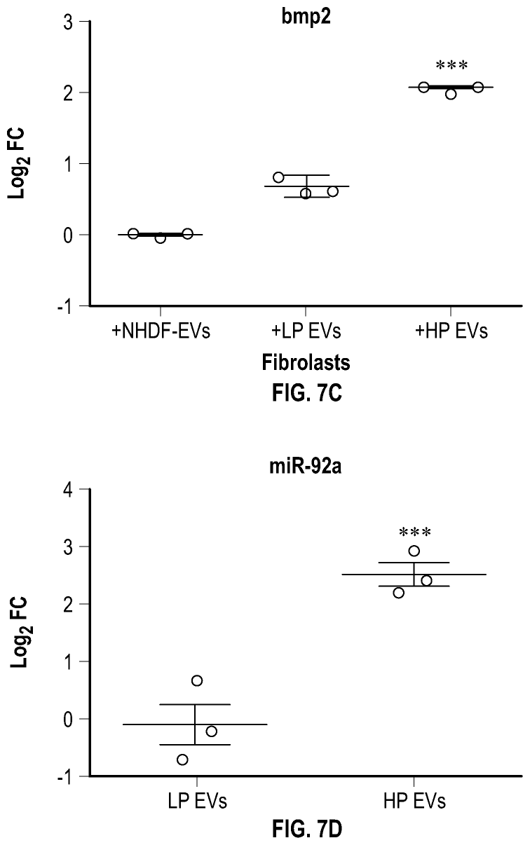

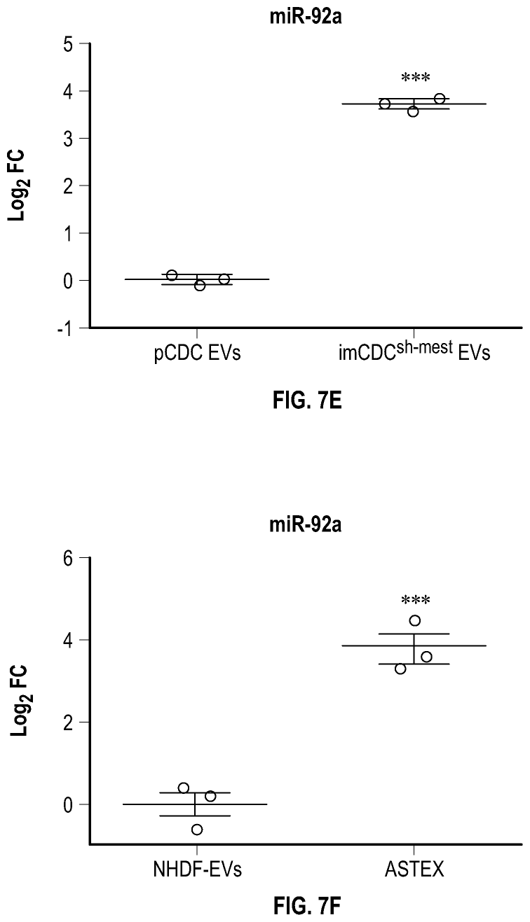

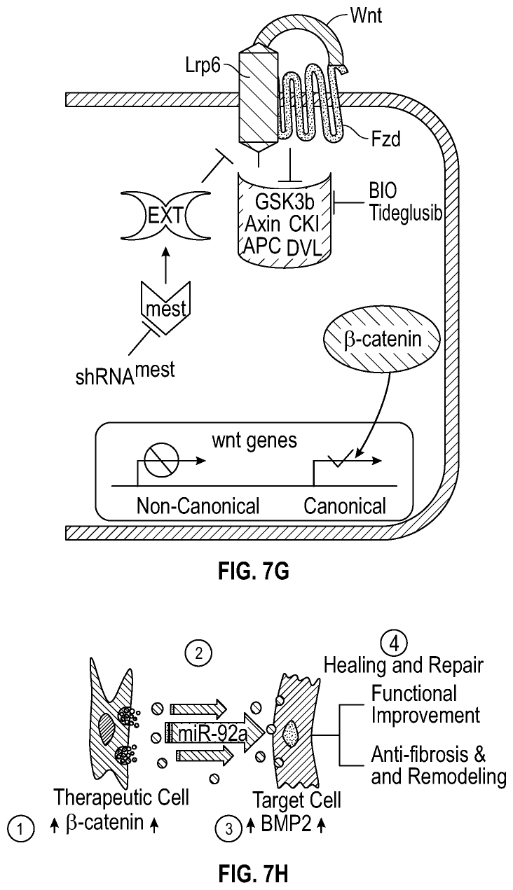

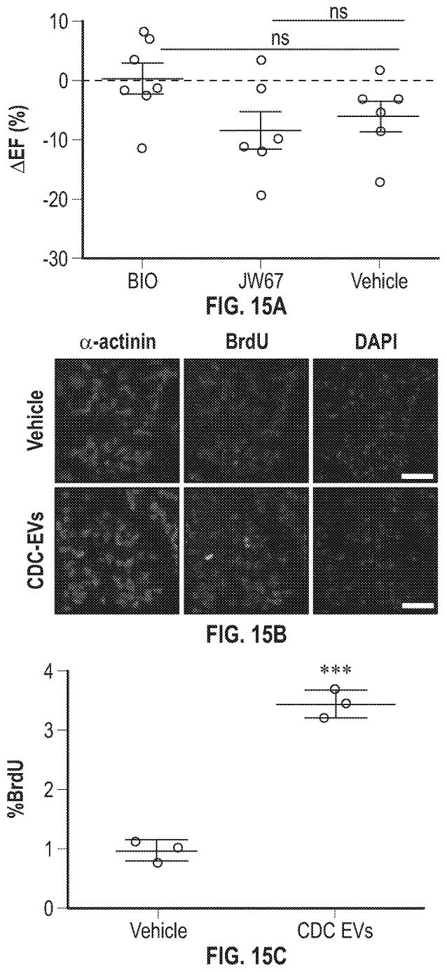

Without wishing to be bound by theory, one theoretical mechanism would posit that β-catenin-activated CDCs simply increased β-catenin levels in the injured myocardium when injected. To test whether myocardial activation of β-catenin is cardioprotective, drugs were administered to alter global canonical Wnt signaling systemically in mice with MI, independent of CDCs. Neither BIO nor the canonical Wnt inhibitor JW67 significantly altered myocardial function relative to controls (FIG. 15A), divorcing global myocardial alterations in Wnt signaling from the effects of CDCs. Instead, transcriptomic analysis in a reductionist in vitro model (using neonatal rat ventricular myocytes; FIGS. 15B and 15C) revealed major changes in the bone morphogenic peptide (BMP) family of genes after exposure to HP CDC EVs. BMP genes are central regulators of cardiac fibrosis; moreover, bmp2 is a target of β-catenin and promotes myocyte contractility and wound healing. Differentially-expressed BMP family members include anti-fibrotic bmp-2, its receptor (2r), -6, and 8a, all of which were upregulated, while profibrotic members, including bmp-3, -4, GDF6, and 10, were suppressed (FIGS. 7A, 7B). Furthermore, fibroblasts exposed to HP EVs upregulate bmp2 compared to fibroblasts exposed to their own EVs or LP-EVs (FIG. 7C). A microRNA coregulated with bmp2, miR-92a, promotes bmp2 signaling. Indeed miR-92a is enriched in HP EVs compared to LP EVs (FIG. 7D). Consistently, miR-92a is also enriched in the EVs of imCDC^{shmest} as well as ASTEX (FIGS. 7E, 7F).

In some embodiments, exposure to HP CDC EVs modulates expression of the bone morphogenic peptide (BMP) family of genes. In some embodiments, bmp-2, its receptor (2r), -6, -8a, or any combination thereof, are upregulated upon exposure to HP CDC EVs. In some embodiments, bmp-3, -4, GDF6, GDF10, or any combination thereof, are suppressed upon exposure to HP CDC EVs. In some embodiments, bmp-2, its receptor (2r), -6, -8a, or any combination thereof, are upregulated and bmp-3, -4, GDF6, GDF10, or any combination thereof, are suppressed upon exposure to HP CDC EVs. In some embodiments, miR-92a is enriched in HP EVs compared to LP EVs. In some embodiments, miR-92a is enriched in HP EVs compared to LP EVs, correlating with upregulation of bmp-2 in cells exposed to HP EVs. In some embodiments, upregulation of bmp-2, its receptor (2r), -6, -8a, or any combination thereof, promotes wound healing and/or tissue repair. In some embodiments, downregulation of bmp-3, -4, GDF6, GDF10, or any combination thereof, promotes wound healing and/or tissue repair. In some embodiments, upregulation of bmp-2, its receptor (2r), -6, -8a, or any combination thereof, and downregulation of bmp-3, -4, GDF6, GDF10, or any combination thereof, promotes wound healing and/or tissue repair.

### Example 7

This non-limiting example shows the engineering of high potency, next generation cell-free therapeutic candidates.

Cardiosphere-derived cells (CDCs) are therapeutic candidates with disease-modifying bioactivity, but, as with all primary cells, variable potency complicates clinical development. Transcriptomic comparison of high- or low-potency CDCs from various human donors revealed activation of Wnt/β-catenin signaling in high-potency CDCs and enrichment of non-canonical Wnt signaling targets in low-potency CDCs. β-catenin protein levels correlated strongly with therapeutic potency, while reconstituting β-catenin in low-potency CDCs restored therapeutic efficacy. The mesoderm-specific transcript mest was downregulated in β-catenin-overexpressing CDCs; in otherwise-inert immortalized CDCs, suppression of mest boosted β-catenin levels and restored potency. To probe the universality of β-catenin as a determinant of disease-modifying bioactivity, skin fibroblasts were studied. Such cells naturally lack potency, but they became immortal and therapeutically-potent when engineered to overexpress β-catenin (and the transcription factor gata4). Both the engineered fibroblasts themselves, and their secreted exosomes, decreased mortality and improved cardiac function in mice with myocardial infarction. In the mdx mouse model of Duchenne muscular dystrophy, exosomes secreted by engineered fibroblasts improved exercise capacity and reduced skeletal muscle fibrosis. Exosomes from high-potency CDCs exhibit enhanced levels of miR-92a, a known potentiator of Wnt/β-catenin, and activate cardioprotective bmp signaling in target cardiomyocytes. Thus, without being limited by theory, canonical Wnt signaling is a manipulable determinant of therapeutic potency in multiple mammalian cell types.

These data show that exosomes from novel immortal cell lines, engineered for high potency, represent next-generation cell-free therapeutic candidates. In some embodiments, cell lines engineered for high potency overexpress β-catenin. In some embodiments, cell lines engineered for high potency overexpress gata4. In some embodiments, cell lines engineered for high potency overexpress β-catenin and gata4.

### Example 8

This non-limiting example shows the role of Wnt signaling in the generation of therapeutically-beneficial engineered novel cell entities (ASTECs) by manipulating β-catenin.

Wnt signaling comprises three highly evolutionarily-conserved pathways; one canonical, which regulates transcription, and two non-canonical, which regulate cell structure and calcium handling. As disclosed herein, canonical Wnt signaling is enriched in potent CDCs, whereas non-canonical Wnt signaling is enriched in non-potent CDCs. β-catenin, which is the nodal point of canonical Wnt signaling, is known to be involved in endometrial regeneration. During the healing phase, β-catenin subsides and CD90 levels increase in stromal tissue. β-catenin signaling figures prominently in a number of related pathophysiological pathways including pro-reparative macrophage polarity, attenuation of fibrosis, cardiomyogenesis, and angiogenesis. Furthermore, cardiac preconditioning is associated with accumulation of β-catenin and its downstream cascade. β-catenin overexpression reduces MI size through effects on cardiomyocytes and cardiac fibroblasts. Without being limited by theory, β-catenin is not only a potency marker but plays a mechanistic role in therapeutic efficacy. Without being limited by theory, mest is an important turning point to non-canonical Wnt signaling through regulation of LRP5/6 expression and activation of EXTL1 (FIG. 7G). β-catenin transcriptionally inhibits mest and extl1, likely through the activity of downstream gene targets, though the exact mechanism remains unknown.

According to several embodiments, activation of β-catenin in CDCs leads to enrichment of its coregulated miR, miR-92a, which in turn leads to improved contractility and attenuation of fibrosis in target tissue (FIG. 7h). The present findings motivate further mechanistic dissection, including elucidation of how β-catenin represses the mest-extl1 axis. As disclosed herein, the role of canonical Wnt signaling can be extended beyond CDCs. By way of non-limiting example, as disclosed herein, deleterious fibroblasts were successfully converted into therapeutically-beneficial engineered novel cell entities (ASTECs) by manipulating β-catenin. The mechanistic findings on CDC potency informed efforts to create ASTECs: immortal, defined cells engineered to have disease-modifying bioactivity. Without being limited by theory, from a product development viewpoint, ASTECs are notable not only because such cells may, themselves, be viable therapeutic candidates, but also because they constitute a well-defined, immortal source for manufacturing high-potency exosomes and other EVs. As reviewed, EVs offer the potential to overcome key limitations of cell therapy. Cells are sensitive and labile living entities, vulnerable to even to minor changes in manufacturing conditions. This renders their manufacturing and scalability costly and logistically burdensome. EVs are non-living, stable, and hardy. As small bilayer vesicles, they can tolerate lyophilization, repeated freeze-thaw cycles, and other harsh handling methods whilst remaining bioactive. Another advantage of their size is the safety of higher dose thresholds and broader penetration into tissue (e.g., crossing the blood-brain barrier) without the concern of microvascular occlusion or viability loss. Furthermore, EVs, unlike their parent cells, exhibit immune versatility, exerting their therapeutic effects even in xenogeneic contexts. Human exosomes have been shown to induce therapeutic benefits in mice, rats, and pigs. ASTEX have all these theoretical advantages. Unlike previous efforts to derive EVs from immortalized cells, ASTEX further have the distinction of having been created by mechanistically-informed genetic engineering of the parent cells to enhance their therapeutic efficacy.

These data show that manipulation of β-catenin results in the generation of therapeutically-beneficial engineered novel cell entities (ASTECs) as a source for high-potency exosomes and other EVs (ASTEX). In some embodiments, engineered novel cell entities (ASTECs) as a source for high-potency exosomes and other EVs (ASTEX) show upregulated or overexpressed β-catenin. In some embodiments, upregulated or overexpressed β-catenin in engineered novel cell entities (ASTECs) as a source for high-potency exosomes and other EVs (ASTEX) inhibits mest, upregulates LRP5/6 expression, inhibits extl1, upregulates miR-92a, or any combination thereof. In some embodiments, mest inhibition, LRP5/6 upregulation, extl1 inhibition, miR-92a upregulation, or any combination thereof, are achieved by gene editing using, for example CRISPR-Cas, zinc finger nucleases, and/or TALENs. In some embodiments, treatment of target cells or target tissues with ASTECs or ASTEX modulates gene expression of the bone morphogenic peptide (BMP) family of genes. In some embodiments, bmp-2, its receptor (2r), -6, and 8a are upregulated upon exposure to ASTECs or ASTEX. In some embodiments, bmp-3, -4, GDF6, and GDF10 are suppressed upon exposure to ASTEC or ASTEX. In some embodiments, bmp-2, its receptor (2r), -6, and 8a are upregulated and bmp-3, -4, GDF6, and GDF10 are suppressed upon exposure to ASTEC or ASTEX.

### Example 9

This non-limiting example shows therapeutic potency of exosomes from immortalized CDCs (imCDC^{sh-mest}).

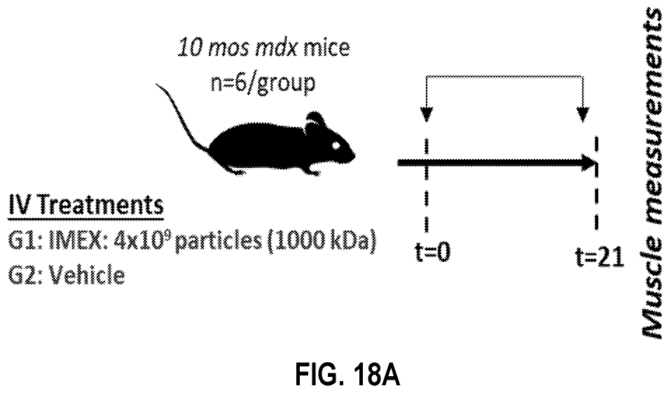

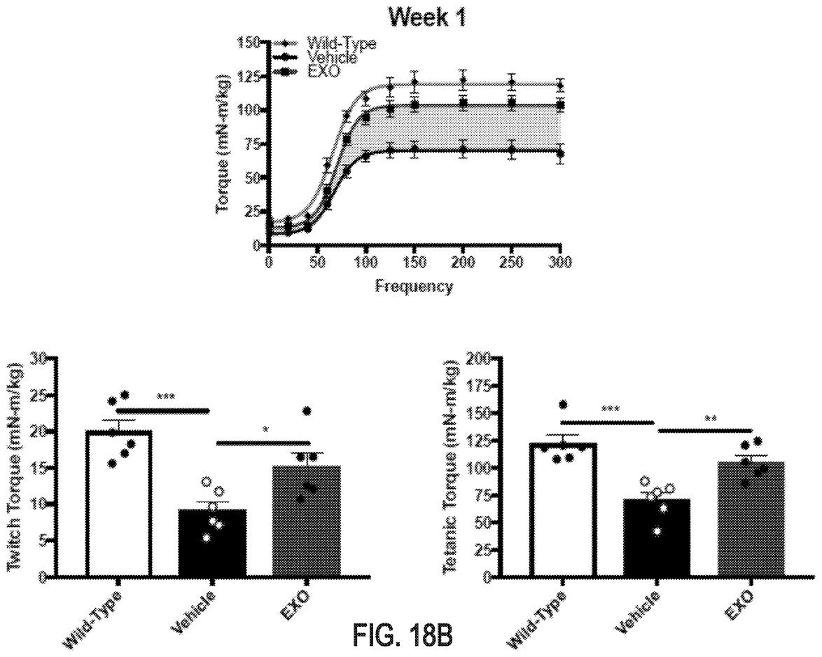

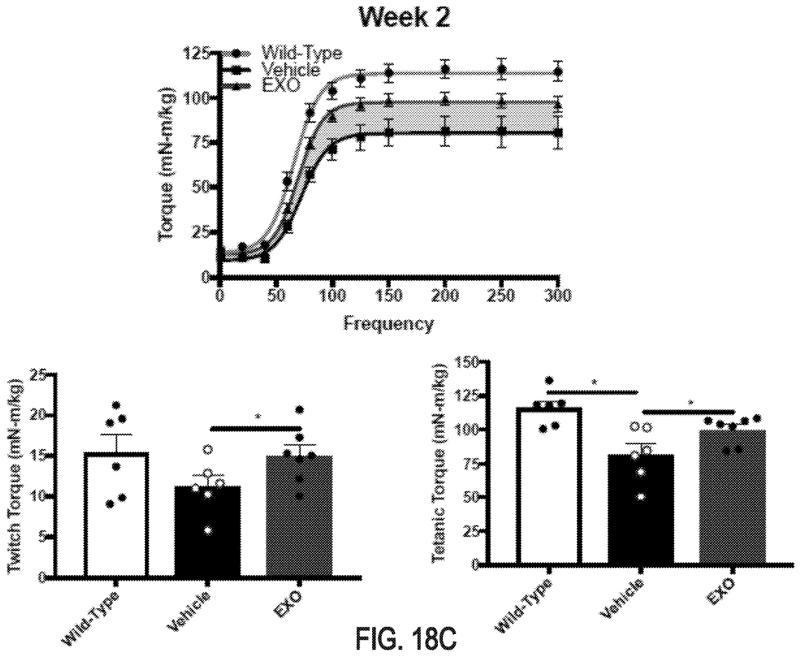

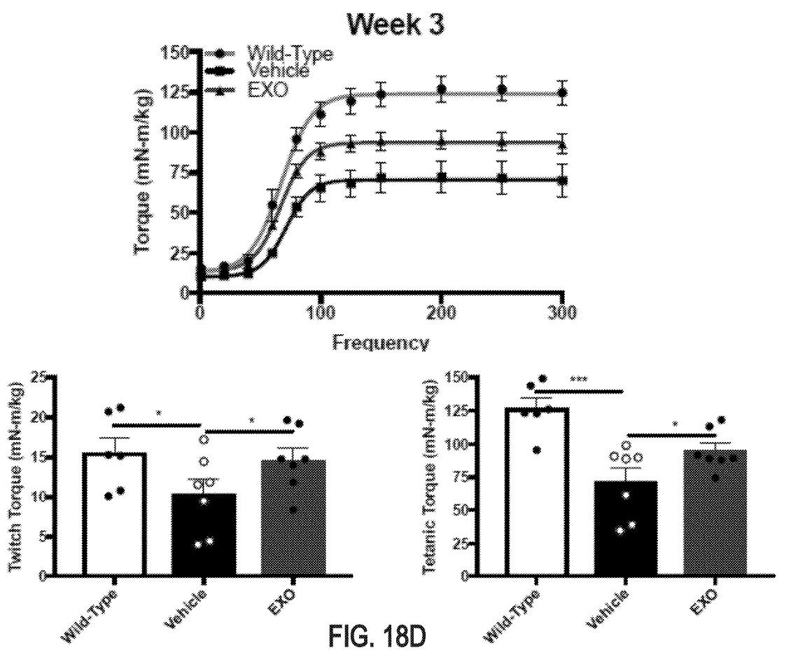

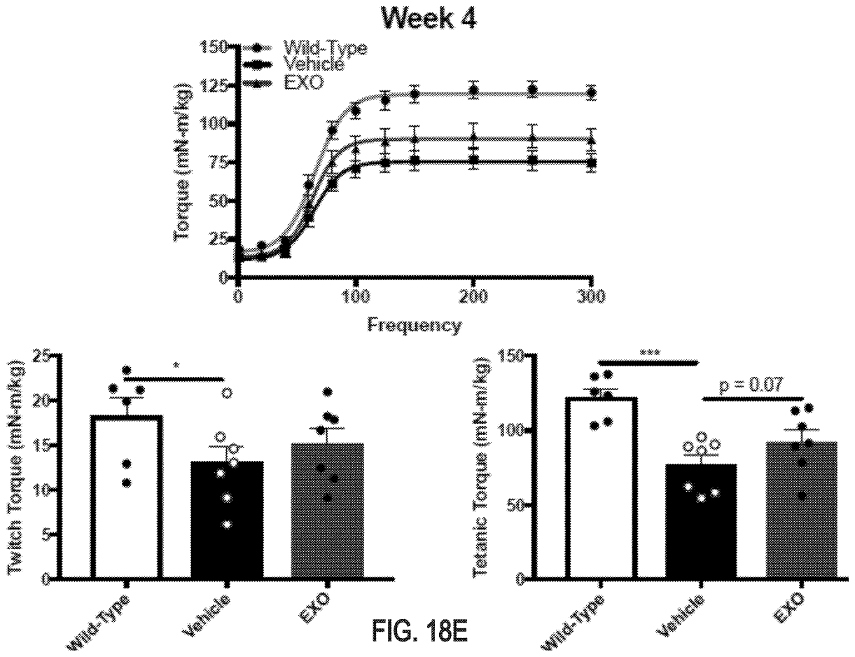

The therapeutic potency of exosomes derived from imCDC^{sh-mest} tested in mdx mice by intravenously injecting 4×10⁹ particles exosomes (IMEX), or vehicle only (FIG. 18A). Muscle force of the tibialis anterior was tested 1 week (FIG. 18B), 2 weeks (FIG. 18C), 3 weeks (FIG. 18D), and 4 weeks (FIG. 18E) after administration. Both twitch and tetanic torque improved in animals administered with the exosomes (EXO) compared to vehicle control for up to three weeks (FIG. 18B-18D). By Week 4, the twitch torque in exosome-treated and vehicle-treated animals were similar, while the tetanique torque in exosome-treated animals showed a higher trend compared to vehicle-treated animals (FIG. 18E).

In some embodiments, administering high potency exosomes derived from high therapeutic potency, immortalized CDCs restores skeletal muscle function in muscular dystrophy (or other skeletal muscle disorders). In some embodiments, a single dose of high potency exosomes derived from high therapeutic potency, immortalized CDCs restores skeletal muscle function in muscular dystrophy (or other skeletal muscle disorders).

### Example 10

This non-limiting example shows exosomal surface marker expression in immortalized CDC (imCDC^{sh-mest})-derived exosomes (IMEX) and ASTEX.

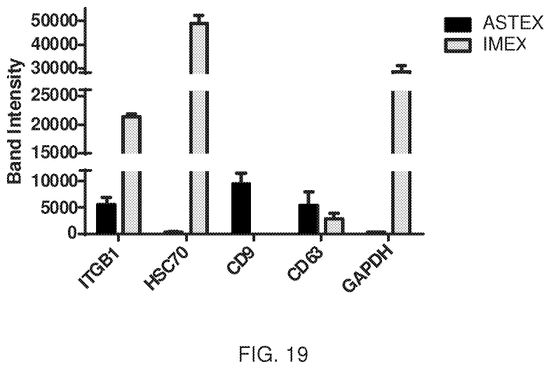

Expression of exosomal surface markers was studied in immortalized CDCs (imCDC^{sh-mest})-derived and ASTEX prepared as described above using Western blotting. ASTEX expressed the surface markers ITGB1, CD9, and CD63, while there was very little expression of HSC70 and GAPDH (FIG. 19). IMEX expressed elevated levels of ITGB1, HSC70, GAPDH, expressed moderate level of CD63, but did not express CD9 (FIG. 19).

In some embodiments, immortalized-CDC-derived exosomes, e.g., immortalized-CDC-derived exosomes having enhanced therapeutic potency, express HSC70, ITGB1, and GAPDH. In some embodiments, immortalized-CDC-derived exosomes, e.g., immortalized-CDC-derived exosomes having enhanced therapeutic potency, express HSC70, ITGB1, GAPDH, and CD63. In some embodiments, immortalized-CDC-derived exosomes, e.g., immortalized-CDC-derived exosomes having enhanced therapeutic potency, do not express CD9. In some embodiments, ASTEX express ITGB1, CD9 and CD63. In some embodiments, ASTEX are depleted for HSC70 and GAPDH.

Although the foregoing has been described in some detail by way of illustrations and examples for purposes of clarity and understanding, it will be understood by those of skill in the art that modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the embodiments of the invention(s).

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering an antigen-binding protein” include “instructing the administration of an antigen-binding protein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

The indefinite article “a” or “an” does not exclude a plurality. The term “about” as used herein to, for example, define the values and ranges of molecular weights means that the indicated values and/or range limits can vary within +20%, e.g., within +10%. The use of “about” before a number includes the number itself. For example, “about 5” provides express support for “5”. Numbers provided in ranges include overlapping ranges and integers in between; for example a range of 1-4 and 5-7 includes for example, 1-7, 1-6, 1-5, 2-5, 2-7, 4-7, 1, 2, 3, 4, 5, 6 and 7.

What is claimed is:

1. A method of preparing therapeutic cells for treating conditions requiring muscle tissue repair, muscle tissue regeneration, or muscle tissue growth, the method comprising overexpressing gata4 in fibroblasts, thereby generating therapeutic cells effective for facilitating muscle tissue repair, muscle tissue regeneration, or muscle tissue growth.

2. The method of claim 1, wherein the fibroblasts are genetically modified to produce the therapeutic cells having higher mRNA expression of gata4 by a log₂ fold of about 0.2 to about 4 relative to fibroblast cells that do not overexpress gata4.

3. The method of claim 1, wherein the fibroblasts are immortalized fibroblasts.

4. The method of claim 1, wherein the fibroblasts are allogeneic to a subject in need of treating a condition requiring the muscle tissue repair, muscle tissue regeneration, or muscle tissue growth.

5. The method of claim 1, wherein the fibroblasts are autologous to a subject in need of treating a condition requiring the muscle tissue repair, muscle tissue regeneration, or muscle tissue growth.

6. The method of claim 1, further comprising isolating exosomes from the therapeutic cells, wherein the exosomes are effective for facilitating muscle tissue repair, muscle tissue regeneration, or muscle tissue growth.

7. The method of claim 1, wherein the therapeutic cells are effective for one or more of reducing cardiac scar size, increasing myocardial infarct wall thickness, increasing ejection fraction, reducing mortality from myocardial infarction, increasing exercise capacity, reducing skeletal muscle fibrosis, and increasing myofiber size, when administered to a subject in need of treating a condition requiring muscle tissue repair, muscle tissue regeneration, or muscle tissue growth.

8. A method of preparing therapeutic exosomes for treating conditions requiring muscle tissue repair, muscle tissue regeneration, or muscle tissue growth, the method comprising:
preparing therapeutic cells by overexpressing gata4 in fibroblasts; and
collecting exosomes from the therapeutic cells,
to thereby generate therapeutic exosomes, wherein the therapeutic exosomes are effective for facilitating muscle tissue repair, muscle tissue regeneration, or muscle tissue growth.

9. The method of claim 8, wherein the therapeutic exosomes comprise increased levels of miR-92a, increased levels of miR-146a, decreased levels of miR-199b, or combinations thereof.

10. A method of treating dystrophic skeletal muscle in a subject with muscular dystrophy, comprising administering to the subject therapeutic cells prepared by overexpressing gata4 in fibroblasts, wherein the therapeutic cells are effective for facilitating muscle tissue repair, muscle tissue regeneration, or muscle tissue growth.

11. The method of claim 10, wherein the fibroblasts are genetically modified to produce the therapeutic cells having higher mRNA expression of gata4 by a log₂ fold of about 0.2 to about 4 relative to fibroblast cells that do not overexpress gata4.

12. A method of treating dystrophic skeletal muscle in a subject with muscular dystrophy, comprising administering to the subject therapeutic exosomes prepared by:
(a) preparing therapeutic cells by overexpressing gata4 in fibroblasts; and
(b) collecting exosomes from the therapeutic cells,
wherein therapeutic potency cells are cardiosphere-derived cells (CDCs) that express less than 30 ng/μL β-catenin, and high potency cells are CDCs that express 30 ng/μL or more β-catenin.

13. The method of claim 12, wherein the therapeutic exosomes comprise increased levels of miR-92a, increased levels of miR-146a, decreased levels of miR-199b, or combinations thereof.

14. The method of claim 10, wherein the fibroblasts are immortalized.

15. The method of claim 14, wherein the fibroblasts are immortalized using simian virus 40 large and small antigens (SV40 T+t).

16. The method of claim 12, wherein the fibroblasts are immortalized.

17. The method of claim 16, wherein the fibroblasts are immortalized using simian virus 40 large and small antigens (SV40 T+t).

18. A population of cells engineered for enhanced therapeutic potency for use in treating damaged or diseased tissue, comprising overexpressing gata4 in fibroblasts, thereby generating cells with enhanced therapeutic potency.

19. A plurality of engineered therapeutic exosomes for use in treating damaged or diseased tissue, wherein said plurality of engineered therapeutic exosomes is obtained by (a) preparing therapeutic cells by overexpressing gata4 in fibroblasts; and (b) collecting exosomes from the therapeutic cells.

20. The plurality of engineered therapeutic exosomes of claim 19, wherein the engineered therapeutic exosomes comprise increased miR-92a relative to low therapeutic potency exosomes, increased miR-146a relative to low therapeutic potency exosomes, and/or reduced miR-199b relative to low therapeutic potency exosomes.
